(19) United States
(12) Patent Application Publication
(10) Pub. No.: US 2018/0043008 A1
(43) Pub. Date: Feb. 15, 2018

(54) RECALL ANTIGEN FOR PROMOTING T-HELPER TYPE 1 RESPONSE

(71) Applicant: Bioventures, LLC, Little Rock, AR (US)

(72) Inventor: Mayumi Nakagawa, Little Rock, AR (US)

(21) Appl. No.: 15/552,285

(22) PCT Filed: Feb. 26, 2016
(86) PCT No.: PCT/US16/19719

§ 371 Date: Aug. 20, 2017

Related U.S. Application Data
(60) Provisional application No. 62/120,978, filed on Feb. 26, 2015.
(60) Provisional application No. 62/271,124, filed on Dec. 22, 2015.

(51) Int. Cl. A61K 39/39; A61K 39/12; A61K 45/06

(52) U.S. Cl. CPC A61K 39/39; A61K 45/06; A61K 39/12; A61K 2039/55588

(57) ABSTRACT

Provided herein is a method of stimulating a systemic T helper cell type 1 response in a person in need thereof, the method comprising: injecting a composition comprising a recall antigen intradermally in a person in need thereof; wherein the method is not a method of treating a herpes simplex virus infection; and wherein the method does not comprise injecting a composition comprising a recall antigen intradermally into a viral epithelial lesion; and (i) wherein the person is infected with a microorganism and afflicted with a disease caused by the microorganism, and the composition comprising a recall antigen does not comprise an antigen of the microorganism infecting the person; or (ii) wherein the person is afflicted with a cancer, and the composition comprising a recall antigen does not comprise an antigen of the cancer afflicting the person.

This invention was made with government support under grant numbers R01CA143130, UL1TR000039, and P20GM103625 awarded by the National Institutes of Health. The government has certain rights in the invention.

### BACKGROUND OF THE INVENTION

Cervical cancer is the fourth most common cancer in women worldwide, with an annual incidence of 528,000 cases and mortality of 266,000 cases. Every year in the United States, there are 12,360 new cases of cervical cancer and 4,020 deaths. High-risk Human Papilloma virus, the most common type being HPV16, is the major cause of cervical cancer. Among the over one hundred different types of Human Papilloma virus, at least 15 are strongly associated with invasive squamous cell cancer of the cervix. HPV16 is the one most commonly found associated with this cancer.

Human Papilloma virus infection is also associated with the precursor lesion of cervical cancer, squamous intraepithelial lesion. While most low-grade squamous intraepithelial lesions prospectively regress spontaneously, some progress to high-grade squamous intraepithelial lesions. These high-grade lesions, in particular cervical intraepithelial neoplasia-3, are associated with a high rate progression to invasive cervical cancer.

Two early gene products, E6 and E7, mediate transformation to a malignant phenotype by Human Papilloma virus. Both of these viral proteins have been shown to interact with the products of cellular human tumor suppressor genes. The E6 protein can bind and promote degradation of cell-encoded p53, while the E7 protein interacts with the retinoblastoma susceptibility gene product. Constitutive expression of HPV E6/E7 proteins is required for the maintenance of a malignant phenotype of cervical cancer.

Cell-mediated immunity plays an important role in controlling Human Papilloma virus infection and Human Papilloma virus-associated diseases. CD4 T cells are important in the development of anti-tumor responses. It is believed that the effectiveness of these CD4 T cells lies in their ability to deliver help for priming and maintaining CD8 cytotoxic T lymphocytes, which are thought to serve as the dominant effector cells in tumor elimination. Immunohistochemical analyses of squamous intraepithelial lesions and cervical cancer specimens have demonstrated the presence of activated cytotoxic T lymphocytes in lesions. The CD4 T cells activate cytotoxic T lymphocytes by producing T helper 1 cytokines and by providing activation signals for priming of tumor-specific cytotoxic T lymphocytes to professional antigen presenting cells. CD8-positive cytotoxic T lymphocytes recognize foreign peptides that are 8 to 11 amino acids in length and bound to and presented by Human Leukocyte Antigen class I molecules. These peptides are called T cell epitopes.

Memory T cells play an important role in maintaining long-term immunity to previously encountered pathogens or tumor antigens. They may proliferate, and rapidly acquire effector functions to kill virus-infected cells or tumor cells, and secrete cytokines that inhibit replication of the pathogen after re-stimulation with re-exposure to antigen. Antigen presenting cells, which may transfer peripheral antigenic signals to the lymphoid organs, play a crucial role in the induction of antigen-specific T cell immunity responses to Human Papilloma virus infection and Human Papilloma virus-associated tumors. Dendritic cells as professional antigen-presenting cells express high levels of major histocompatibility complex and co-stimulatory molecules. Insufficient or improper activation of dendritic cells, caused by lack of pro-inflammatory signal, leading to antigen presentation not in an appropriate co-stimulatory context is one reason for the failure of antitumor immunity.

Prophylactic HPV vaccines are available, and work by preventing HPV infection. But they are not effective in individuals who are already infected. An HPV therapeutic vaccine would benefit women who have pre-cancerous lesions but wish to have children since standard surgical treatments are associated with increased risk for pre-term delivery. It would also benefit women and men who live in developing regions of the world and do not have access to surgical modalities.

Treatments that would improve immune system control of other diseases, including viral, fungal, and bacterial infections, and cancer are also needed.

### SUMMARY

Pharmaceutical formulations containing HPV peptides for use as therapeutic vaccines are provided. Also provided is a method of making the formulations, especially a method of solubilizing a difficult-to-solubilize HPV peptide. Also provided are methods of treating HPV infection and HPV-associated lesions, including HPV-associated cancers.

One embodiment provides a method to solubilize an HPV E6 peptide comprising: solubilizing an HPV E6 peptide A of 20 to 100 amino acids in length and comprising at least 20 consecutive residues of HPV E6 81-115 (residues 81-115 of SEQ ID NO:1) in a buffer that before the step of solubilizing the HPV peptide A contains in dissolved form two or more HPV peptides Y of 10 to 100 amino acids in length each that collectively comprise at least 50% of the sequence of HPV E6 1-80 (residues 1-80 of SEQ ID NO:1) and at least 50% of HPV E6 116-158 (residues 116-158 of SEQ ID NO:1) to create a final soluble composition containing the peptide A in dissolved form and the peptides Y in dissolved form. The peptides Y in the buffer before the step of solubilizing the peptide A are preferably in fully dissolved form (no insoluble peptides Y) and in the final soluble composition the peptides A and Y are preferably in fully dissolved form.

Another embodiment provides a pharmaceutical formulation comprising: (a) one or more HPV E6 peptides, each of a length of 10-100 amino acid residues; (b) glutamate at a concentration of 2-40 mM; (c) trehalose at a concentration of 0.3% to 5% w/v; (d) glycine at a concentration of 0.2% to 10% w/v; wherein the formulation is at a pH of 3.0 to 5.0.

Another embodiment provides a pharmaceutical formulation comprising: an HPV E6 peptide A and one or more HPV peptides Y, the composition made by a method comprising: solubilizing an HPV E6 peptide A of 20 to 100 amino acids in length and comprising at least 20 consecutive residues of HPV E6 81-115 (residues 81-115 of SEQ ID NO:1) in a buffer that before the step of solubilizing the HPV peptide A contains in dissolved form two or more HPV peptides Y of 10 to 100 amino acids in length each that collectively comprise at least 50% of the sequence of HPV E6 1-80 (residues 1-80 of SEQ ID NO:1) and at least 50% of HPV E6 116-158 (residues 116-158 of SEQ ID NO:1) to create a final soluble composition containing the peptide A in dissolved form and the peptides Y in dissolved form.

Another embodiment provides a method of decreasing infection from human papilloma virus (HPV) in an individual or increasing regression of HPV-associated lesions in an HPV-positive individual, comprising: administering a pharmaceutical formulation comprising (a) one or more HPV E6 peptides, each of a length of 10-100 amino acid residues; (b) glutamate at a concentration of 2-40 mM; (c) trehalose at a concentration of 0.3% to 5% w/v; (d) glycine at a concentration of 0.2% to 10% w/v.

It is shown herein in Example 2 that recall antigens, such as CANDIN, enhance the T cell immune response to the HPV peptides tested. A combination of a recall antigen and HPV peptides was contacted with peripheral blood mononuclear cells in Example 2. Thus, administering a vaccine that includes a recall antigen together with disease-specific antigens may have general applicability to promote a cellular (T cell) immune response to the disease-specific antigens.

Accordingly, one embodiment provides a method of decreasing infection from human papilloma virus (HPV) in an individual or increasing regression of HPV-associated lesions in an HPV-positive individual, to induce a T cell response to HPV, the method comprising: administering to the individual a composition comprising one or more HPV antigens and administering to the individual a recall antigen that is not an HPV antigen; wherein the recall antigen is administered to be in contact with the one or more HPV antigens in the individual; wherein the individual is in need of a T cell response against the one or more HPV antigens; wherein the one or more HPV antigens are not E6 antigens.

In a Phase I clinical trial of women with biopsy-proven high-grade squamous intraepithelial lesion (HSIL), women were treated with intradermal injection of a composition comprising HPV protein E6 residues 1-45 (SEQ ID NO:2), E6 46-80 (SEQ ID NO:3), E6 81-115 (SEQ ID NO:4), and E6 116-158 (SEQ ID NO:5), all mixed with CANDIN as an adjuvant. The dosages tested were 50 ug, 100 ug, and 250 ug of each of the peptides. It was surprisingly found that 5 of 6 subjects (83%) in the 50 ug dose group, 3 of 6 subjects (50%) in the 100 ug dose group, 2 of 6 (33%) subjects in the 250 ug dose group, and 2 of 5 (40%) in the 500 ug dose group had complete or partial responses. The complete response rates (no HSIL remaining) were 4/6, 3/6, 1/6, and 1/5 in the 50, 100, 250, and 500 ug dose groups respectively. This is a surprising result that the lowest dose was the most effective. This is reported in Example 3 below.

Thus, another embodiment provides a unit dosage pharmaceutical composition comprising: 25 to 110 ug of a peptide consisting of SEQ ID NO:2, 25 to 110 ug of a peptide consisting of SEQ ID NO:3, 25 to 110 ug of a peptide consisting of SEQ ID NO:4, 25 to 110 ug of a peptide consisting of SEQ ID NO:5; and a recall antigen; in a unit dosage form for intradermal injection in a volume of 100 to 900 ul.

Another embodiment provides a method of treating HPV infection comprising: injecting into a patient intradermally a unit dosage pharmaceutical composition comprising: 25 to 110 ug of a peptide consisting of SEQ ID NO:2, 25 to 110 ug of a peptide consisting of SEQ ID NO:3, 25 to 110 ug of a peptide consisting of SEQ ID NO:4, 25 to 110 ug of a peptide consisting of SEQ ID NO:5; and a recall antigen; in a unit dosage form for intradermal injection in a volume of 100 to 900 ul.

Another embodiment provides a method of treating a disease caused by microorganism in a mammalian subject comprising: administering intradermally to the subject a composition comprising one or more antigens of the microorganism and administering intradermally to the subject a recall antigen that is not an antigen of the microorganism; wherein the recall antigen is administered to be in contact with the one or more antigens of the microorganism in the subject.

It is shown in Example 1 below that CANDIN alone induces Interleukin-12 (IL-12) secretion by Langerhans cells in vitro when CANDIN is contacted with the Langerhans cells. IL-12 stimulates Th1 T helper cell subpopulation, so it seemed possible that CANDIN would stimulate proliferation of Th1 cells. This has now been found in a Phase I human trial involving intradermal injection of a composition comprising CANDIN and HPV type 16 E6 peptides. The inventor believes that intradermal injection of CANDIN alone will stimulate Th1 cell proliferation in vivo, and this will be beneficial for immune response to microbial infections, including bacterial, viral, and fungal infections, and for anti-cancer immune response.

Thus, one embodiment provides a method of stimulating a systemic T helper cell type 1 response in a person in need thereof, the method comprising: injecting a composition comprising a recall antigen intradermally in a mammal in need thereof; wherein the method is not a method of treating a herpes simplex virus infection; and wherein the method does not comprise injecting a composition comprising a recall antigen intradermally into a viral epithelial lesion; wherein the method increases T helper cell type 1 response in the mammal; and (i) wherein the mammal is infected with a microorganism and afflicted with a disease caused by the microorganism, and the composition comprising a recall antigen does not comprise an antigen of the microorganism infecting the person; or (ii) wherein the mammal is afflicted with a cancer or was afflicted with a cancer and the cancer is now in remission, and the composition comprising a recall antigen does not comprise an antigen of the cancer currently or previously afflicting the mammal.

Another embodiment provides a method of treating a microbial infection or cancer in a mammal comprising: injecting a composition comprising a recall antigen intradermally in a person in need thereof; wherein the method is not a method of treating a herpes simplex virus infection; and wherein the method does not comprise injecting a composition comprising a recall antigen intradermally into a viral epithelial lesion; and (i) wherein the mammal is infected with a microorganism and afflicted with a disease caused by the microorganism, and the composition comprising a recall antigen does not comprise an antigen of the microorganism infecting the mammal, or (ii) wherein the mammal is afflicted with a cancer or was afflicted with a cancer and the cancer is now in remission, and the composition comprising a recall antigen does not comprise an antigen of the cancer currently or previously afflicting the mammal.

Another embodiment provides a method of preventing cancer in a mammal comprising: injecting a composition comprising a recall antigen intradermally in the mammal. In a more specific embodiment, the composition does not comprise an antigen of cancer or an HPV antigen.

Another embodiment provides a method of stimulating a systemic T helper cell type 1 response in a mammal in need thereof, the method comprising: injecting a composition comprising a recall antigen intradermally in a mammal in need thereof; wherein the method is not a method of treating a herpes simplex virus infection; and wherein the method does not comprise injecting a composition comprising a recall antigen intradermally into a viral epithelial lesion; wherein the method increases T helper cell type 1 response in the mammal; and wherein the mammal was afflicted with a cervical cancer or head and neck cancer or a cancer caused by HPV and the cancer is now in remission.

Another embodiment provides a method of preventing growth of tumors or recurrence of cancer in a mammal comprising: injecting a composition comprising a recall antigen intradermally in a mammal in need thereof; wherein the method is not a method of treating a herpes simplex virus infection; and wherein the method does not comprise injecting a composition comprising a recall antigen intradermally into a viral epithelial lesion; wherein the method increases T helper cell type 1 response in the mammal; and wherein the mammal is afflicted with cervical cancer or head and neck cancer or a cancer caused by HPV, or the mammal was afflicted with cervical cancer or head and neck cancer or a cancer caused by HPV and the cancer is now in remission.

### BRIEF DESCRIPTION OF THE DRAWINGS

FIGS. 1A-C Surface expressions of CD1a (FIG. 1A), Langerin (FIG. 1B), and E-cadherin (FIG. 1C) show successful conversion to LCs (solid lines). The dotted lines represent the relevant isotype controls.

FIGS. 2A-B Maturation effects on LCs examined by surface expression of CD40, CD80, CD86, and HLA-DR. (FIG. 2A) Representative FACS histograms from subject 2. The shaded gray area, the black dotted line, the black solid line, the short dashed line and the long dashed line represent the isotype control, media, CANDIN, “peptides” and CANDIN/“peptides” respectively. (FIG. 2B) Summary of results from all subjects examined.

FIG. 3 T-cell proliferation measured using alamarBlue. CANDIN and CANDIN/“peptides” pulsed LCs induce significantly increased T-cell proliferation compared to media. All wells contained CD3 T-cells (1.5×10⁵ cells) and autologous LCs (3×10³ cells).

FIG. 4 Representative results of cytokine expression by LCs treated with CANDIN (150 μl/ml) or CANDIN/“peptides” from subject 4 are shown. The bars represent SD of the replicates.

FIGS. 5A-I. Intracellular cytokine staining for IFN-γ, IL-4 and IL-17A of CD4 T-cells stimulated with LCs pulsed with CANDIN or CANDIN/“peptides”. (A) A representative dot plot for subject 1 showing the gating on lymphocytes. (B) A representative dot plot for subject 1 showing gating on live cells discriminated using eFluor 450. (C) A representative dot plot for subject 1 showing IL-4 secreting CD4 cells that were exposed to LCs pulsed with CANDIN/“peptides”. (D) Corresponding isotype control for IL-4. (E) A representative dot plot for subject 1 showing IFN-γ secreting CD4 cells that were exposed to LCs pulsed with CANDIN/“peptides”. (F) Corresponding isotype control for IFN-γ. (G) A representative dot plot showing IL-17A secreting CD4 cells that were exposed to LCs pulsed with CANDIN/“peptides”. (H) Corresponding isotype control for IL-17A. (I) Diagrams summarizing the results from all subjects.

FIG. 6A, circulating immune cells before, after 2, and after 4 vaccinations in all vaccine recipients. FIG. 6B, circulating immune cells in responders () and non-responders (▪). Percentages of CD4 cells positive for CD4 and Tbet were classified as Th1 cells, positive for CD4 and GATA3 were classified as Th2 cells, and positive for CD4, CD25, and FoxP3 were classified as Tregs. The bars represent standard error of means.

FIG. 7. Regulatory T-cells in lesional cervical epithelium and the underlying stroma. FoxP3 nuclear staining cells, in lesions (cervical intraepithelial neoplasia 1, 2, and/or 3) remaining after vaccination or representative region if no lesions remaining, were counted. The FoxP3 nuclear staining cells were also counted in the underlying stroma. The bars represent stand error of means.

FIG. 8. Schematic presentation of study visits scheduled for the Phase II clinical trial of our HPV therapeutic vaccine. Blood tests are for clinical analyses. Blood draws are for scientific analyses. CRSC, Clinical Research Services Core Unit; Colpo, colposcopy, Bx, biopsy, ECC, endocervical curettage, LEEP, loop electrosurgical excision procedure.

### DETAILED DESCRIPTION

One embodiment of the the invention involves HPV peptides for use in a therapeutic vaccine.

Transformation of squamous epithelium to a malignant phenotype by human papilloma virus is mediated by two early gene products—E6 and E7. Both viral proteins have been shown to interact with the products of cellular human tumor-suppressor genes. The E6 protein can bind and promote degradation of cell-encoded p53, whereas the E7 protein interacts with the retinoblastoma susceptibility gene product. Expressions of E6 and E7 open reading frames have been shown to be necessary and sufficient for the transformation of human cells by HPV-16.

We have investigated previously the epitopes of E6 and E7 that are recognized in favorable immune responses to HPV. (Nakagawa, M. et al., 2010, Journal of Lower Genital Tract Disease, Vol. 14, No. 2, p. 124-129; U.S. Patent Publication Nos. 20110293651, 20090136531, 20090117140, 20060182763).

We have identified HPV E6 and E7 peptides for use in therapeutic vaccines, especially HPV E6 peptides (U.S. Patent Publication Nos. 20110293651, 20090136531, 20090117140).

Numerous types of HPV exist. The one most commonly associated with cancer is HPV-16.

The peptides described herein are from the E6 protein of HPV (HPV E6).

The sequence of E6 from HPV-16 is SEQ ID NO:1 below:

```
(SEQ ID NO: 1)
                10 20 30 40
MHQKRTAMFQ DPQERPRKLP QLCTELQTTI HDIILECVYC
                50 60 70 80
KQQLLRREVY DFAFRDLCIV YRDGNPYAVC DKCLKFYSKI
                90 100 110 120
SEYRHYCYSL YGTTLEQQYN KPLCDLLIRC INCQKPLCPE
              130 140 150
EKQRHLDKKQ RFHNIRGRWT GRCMSCCRSS RTRRETQL.
```

The peptides in the following embodiments are HPV E6 peptides, meaning they are derived from the sequence of an HPV E6 protein. The E6 protein can be from any HPV strain. In a preferred embodiment, the peptides are derived from the E6 of HPV-16.

Preferably, the peptides comprise only HPV E6 sequence. But they may comprise other amino acid residues. They may comprise E6 sequence from any HPV strain, not just HPV-16.

The peptides are preferably chemically synthesized, but they may also be produced in a recombinant organism from recombinant DNA technology. They may also be produced by other means known to persons of skill in the art, for instance by proteolysis of E6 or proteolysis of a longer peptide than the peptide produced.

The peptides in some embodiments are acetylated at their amino termini or amidated at their carboxy termini, or both. In other embodiments, neither terminus is modified.

The peptides may be in specific embodiments 10-100, 8-100, 8-75, 8-50, 8-40, 10-75, 10-50, 10-40, 20-100, 20-75, 20-50, 20-40, 30-100, 30-75, 30-50, or 30-40 amino acid residues in length.

The peptides are generally “forward L” meaning that they have the sequence described and the amino acids are L stereoisomers. In specific embodiments, however, the peptides can be reverse D peptides, meaning that the ordinary sequence of amino acid residues is reversed and the amino acids are D stereoisomers.

One embodiment comprises a method to solubilize an HPV E6 peptide comprising: solubilizing an HPV E6 peptide A of 20 to 100 amino acids in length and comprising at least 20 consecutive residues of HPV E6 81-115 (residues 81-115 of SEQ ID NO:1) in a buffer that before the step of solubilizing the HPV peptide A contains in fully dissolved form two or more HPV peptides Y of 10 to 100 amino acids in length each that collectively comprise at least 50% of the sequence of HPV E6 1-80 (residues 1-80 of SEQ ID NO:1) and at least 50% of HPV E6 116-158 (residues 116-158 of SEQ ID NO:1) to create a final soluble composition containing the peptide A in fully dissolved form and the peptides Y in fully dissolved form.

In a specific embodiment, the peptide A is acetylated at its amino terminus and amidated at its carboxyl terminus.

In a specific embodiment, the HPV peptide A comprises residues 81-115 of SEQ ID NO:1. In other embodiments, the HPV peptide A comprises 25 consecutive residues of residues 81-115 of SEQ ID NO:1 or comprises 30 consecutive residues of residues 81-115 of SEQ ID NO:1.

In a specific embodiment, the HPV peptide A consists of residues 81-115 of SEQ ID NO:1.

In specific embodiments, the peptide A is acetylated on its amino terminus and amidated on its carboxyl terminus.

In a specific embodiment, the buffer is at a pH of from about pH 3.0 to about pH 5.0, from about pH 3.5 to about pH 4.5, or from about pH 2.5 to about pH 5.5.

In specific embodiments, the buffer comprises at least 2 mM glutamate. In other embodiments, it may have 2 to 50 mM glutamate, at least 5 mM glutamate, 5 to 50 mM glutamate, or 5 to 25 mM glutamate, or 2 to 25 mM glutamate. The term “glutamate” in this context is intended to include all forms, protonated and unprotonated, of glutamate, i.e., both glutamate and glutamic acid.

In a specific embodiment, the peptides A and Y collectively comprise all of SEQ ID NO:1 or all of an HPV E6 sequence.

In a specific embodiment, peptide A consists of residues 81-115 of SEQ ID NO:1 and the peptides Y are three peptides consisting of residues 1-45, 46-80, and 116-158 of SEQ ID NO:1.

In a more specific embodiment of this, each of the peptides A and Y is acetylated on its amino terminus and amidated on its carboxyl terminus, wherein the buffer is at a pH of from about pH 3.0 to pH 5.0, and after solubilization, peptide A and each of the three peptides Y is at 0.1 to 20 mg/ml concentration. In other embodiments, after solubilization, peptide A and each of the three peptides Y is at 0.1 to 5 mg/ml or 0.02 to 5 mg/ml.

In a specific embodiment, each of the peptides Y is at at least 80% of the weight-to-volume concentration of peptide A in the final soluble composition.

In a specific embodiment, peptide A and each of the peptides Y are at 0.1 to 5 mg/ml in the final soluble composition. In other embodiments, they are at 0.1 to 20 mg/ml, or 0.02 to 5 mg/ml.

One embodiment provides a pharmaceutical composition comprising: (a) one or more HPV E6 peptides, each of a length of 10-100 amino acid residues; (b) glutamate at a concentration of 2-40 mM; (c) trehalose at a concentration of 0.3% to 5% w/v; (d) glycine at a concentration of 0.2% to 10% w/v; wherein the composition has a pH of 3.0 to 5.0.

Other possible ranges of the glutamate concentration are 2 to 20 mM and 5 to 20 mM. Other possible ranges of trehalose concentration are 0.2% to 5% w/v, 0.5% to 5% w/v, and 0.3% to 2% w/v, and 0.5% to 2% w/v. Other possible ranges of glycine concentration are 0.2% or more, 0.3% or more, 0.5% or more, 1% or more, and up to 3%, up to 5%, up to 8%, up to 10% , up to 15%, and up to 20%.

In a specific embodiment, at least one of the one or more HPV E6 peptides comprises residues 46-70 of SEQ ID NO:1 or comprises residues 91-115 of SEQ ID NO:1, or comprises residues 80-88 of SEQ ID NO:1. In a specific embodiment, at least one of the one or more HPV E6 peptides comprises residues 46-70 of SEQ ID NO:1 or comprises residues 91-115 of SEQ ID NO:1.

In a specific embodiment, the pharmaceutical composition comprises at least three HPV E6 peptides each of a length of 10-100 amino acid residues and collectively comprising at least 50% of an HPV E6 sequence.

In specific embodiments, the composition comprises at least one peptide consisting of residues 1-45, 46-80, 81-115, or 116-158 of SEQ ID NO:1; at least two peptides consisting of residues 1-45, 46-80, 81-115, or 116-158 of SEQ ID NO:1; at least three peptides consisting of residues 1-45, 46-80, 81-115, or 116-158 of SEQ ID NO:1, or comprises four peptides consisting respectively of residues 1-45, 46-80, 81-115, and 116-158 of SEQ ID NO:1.

In specific embodiments, each of the peptides is acetylated at its amino terminus and amidated at its carboxy terminus.

The pharmaceutical composition may also comprise a recall antigen. The prototypical recall antigens are those commonly used in immunologic skin testing to test immune response, particularly mumps antigen, candida antigen, and trichophyton antigen. The test shows if the body “remembers” or “recalls” the antigen, i.e., has a delayed-type hypersensitivity response in the skin where the antigen was administered by intradermal injection.

The term “recall antigen” is defined herein as a substance or mixture containing a plurality of proteinaceous antigens, wherein the mixture induces a delayed-type hypersensitivity response in intradermal skin test in a majority of people previously sensitized or exposed to the recall antigen. The prototypical recall antigens are those commonly used in immunologic skin testing to test immune response, particularly mumps antigen, candida antigen, and trichophyton antigen. Each of these, although referred to by the singular term “antigen” is actually composed of several or many molecular substances that can induce an immune response.

In specific embodiments, the recall antigen may be mumps antigen (e.g., killed whole mumps virus), Candida extract, or Trichophyton extract.

In specific embodiments, the recall antigen is killed whole virus, killed whole bacteria, or killed whole microorganisms.

Example 2 below shows that E6 peptides have partial maturation effects on Langerhans cells in vitro, while Candida extract was responsible for T cell proliferation in vitro in cells exposed to the E6 peptides. So the Candida extract is an excellent adjuvant for the E6 peptides to induce a stronger T cell response to HPV.

We are conducting a clinical trial involving intradermal injection of four HPV E6 peptides together with CANDIN. The peptides are in a pharmaceutical solution A containing 10 mM glutamate, 1.0% w/v trehalose, 2.0% w/v glycine, and 0.714 mg/ml for each of four HPV-16 E6 peptides (consisting of residues 1-45, 46-80, 81-115, and 116-158 of SEQ ID NO:1, each amidated at its carboxy terminus and acetylated at its amino terminus). The pharmaceutical solution A is withdrawn into a syringe in the amounts of 50 μg, 100 μg, 250 μg, or 500 μg (70 to 700 μl of solution A) and mixed in the syringe with 300 μl of CANDIN. The mixture in the syringe is then injected intradermally in an HPV-positive patient having cervical lesions.

CANDIN (candida albicans) is made from the culture filtrate and cells of two strains of Candida albicans. The fungi are propagated in a chemically defined medium consisting of inorganic salts, biotin and sucrose. Lyophilized source material is extracted with a solution of 0.25% NaCl, 0.125% NaHCO₃ and 50% v/v glycerol. The concentrated extract is diluted with a solution of 0.5% NaCl, 0.25% NaHCO₃, 0.03% Albumin (Human) usp, 8 ppm polysorbate 80 and 0.4% phenol.

The potency of CANDIN (candida albicans) is measured by DTH skin tests in humans. The procedure involves concurrent (side-by-side) testing of production lots with an Internal Reference (IR), using sensitive adults who have been previously screened and qualified to serve as test subjects. The induration response at 48 hours elicited by 0.1 mL of a production lot is measured and compared to the response elicited by 0.1 mL of the IR. The test is satisfactory if the potency of the production lot does not differ more than ±20% from the potency of the IR, when analyzed by the paired t-test (two-tailed) at a p value of 0.05

The potency of the IR is monitored by DTH skin testing. Persons included in the potency assay are qualified as test subjects by receiving four skin tests with the IR from which a mean induration response (mm) is calculated. Current skin tests with the IR must show that the potency of the IR has not changed more than ±20% from the mean qualifying response in the same test subjects, when analyzed by the paired t-test (two-tailed) at a p value of 0.05. The required induration response at 48 hours to the IR is 15 mm±20%.

The skin-test strength of CANDIN (candida albicans) has been determined from dose-response studies in healthy adults. The product is intended to elicit an induration response ≧5 mm in immunologically competent persons with cellular hypersensitivity to the antigen.

Another embodiment provides a method of decreasing infection from human papilloma virus (HPV) in an individual or increasing regression of HPV-associated lesions in an HPV-positive individual, comprising: administering a pharmaceutical formulation comprising (a) one or more HPV E6 peptides, each of a length of 10-100 amino acid residues; (b) glutamate at a concentration of 2-40 mM; (c) trehalose at a concentration of 0.3% to 5% w/v; (d) glycine at a concentration of 0.2% to 10% w/v.

Another embodiment provides a method of decreasing infection from human papilloma virus (HPV) in an individual or increasing regression of HPV-associated lesions in an HPV-positive individual, comprising: administering the pharmaceutical composition to an HPV-positive individual in need thereof. In this case the pharmaceutical composition may be pharmaceutical composition comprising: an HPV E6 peptide A and one or more HPV peptides Y, the composition made by a method comprising: solubilizing an HPV E6 peptide A of 20 to 100 amino acids in length and comprising at least 20 consecutive residues of HPV E6 81-115 (residues 81-115 of SEQ ID NO:1) in a buffer that before the step of solubilizing the HPV peptide A contains in dissolved form two or more HPV peptides Y of 10 to 100 amino acids in length each that collectively comprise at least 50% of the sequence of HPV E6 1-80 (residues 1-80 of SEQ ID NO:1) and at least 50% of HPV E6 116-158 (residues 116-158 of SEQ ID NO:1) to create a final soluble composition containing the peptide A in dissolved form and the peptides Y in dissolved form.

In specific embodiments of these methods of treatment, the method comprises injecting the pharmaceutical composition intradermally. It may also be administered by other routes, including intravenous or subcutaneous injection, or enterally. But intradermal injection is the preferred route.

In specific embodiments of the methods of treatment, the pharmaceutical composition further comprises a recall antigen.

In specific embodiments of the method of treatment, the method further comprises injecting a recall antigen intradermally.

In specific embodiments, the method is a method of increasing regression of an HPV-associated lesion in an HPV-positive individual, and the lesion is a malignant tumor.

In specific embodiments, the lesion is a cervical carcinoma.

In specific embodiments, the lesion is a head and neck carcinoma.

In specific embodiments, the method is a method of increasing regression of an HPV-associated lesion, and the lesion is a cervical, vulvar, vaginal, penile, anal, or oropharyngeal tumor.

In a specific embodiment, the method is a method of increasing regression of an HPV-associated lesion, and the lesion is a high-grade squamous intraepithelial lesion (HSIL).

In other embodiments, the method is a method of increasing regression of an HPV-associated lesion in an HPV-positive individual, and the lesion is a benign tumor or a precancerous lesion.

The peptides in some embodiments are acetylated at their amino termini or amidated at their carboxy termini, or both. In other embodiments, neither terminus is modified.

Preferably in the method the composition is administered by intradermal injection. But it may be administered by any suitable method, for instance by intramuscular injection.

One embodiment provides a method of decreasing infection from human papilloma virus (HPV) in an individual or increasing regression of HPV-associated lesions in an HPV-positive individual, to induce a T cell response to HPV, the method comprising: administering to the individual a composition comprising one or more HPV antigens and administering to the individual a recall antigen that is not an HPV antigen; wherein the recall antigen is administered to be in contact with the one or more HPV antigens in the individual; wherein the individual is in need of a T cell response against the one or more HPV antigens. In specific embodiments, the one or more HPV antigens are E6 antigens or E7 antigens. In other specific embodiments, they are not E6 antigens. In another specific embodiment, they are not E7 antigens.

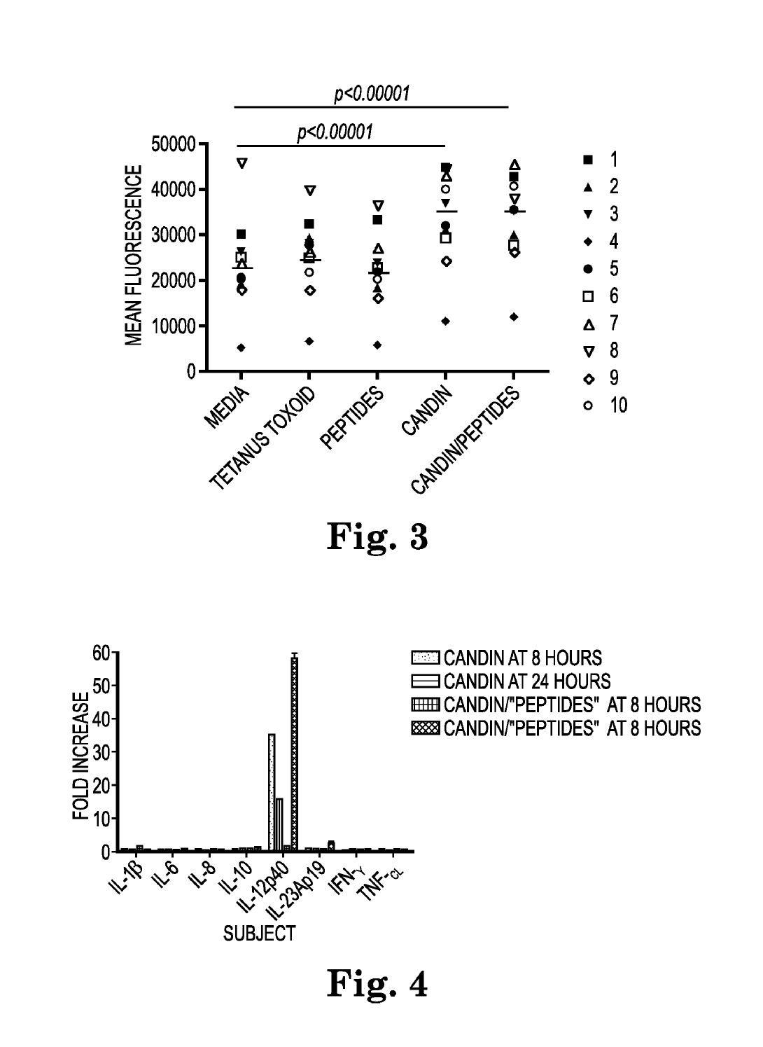

The method is expected to generate a stronger T cell response against the HPV antigens in the individual administering than an otherwise identical method that does not comprise administering a recall antigen that is not an HPV antigen. “Stronger T cell response” may be shown for example by greater antigen-specific T-cell mediated cytotoxicity or antigen-specific T cell proliferative response in vitro in T cells from a subject treated with a combination of a recall antigen and disease-specific antigen(s) versus from a subject treated with the disease-specific antigen(s) without the recall antigen. This can be demonstrated by testing of human subjects in a clinical trial or more likely in animal model testing, or by in vitro testing of T cells from a person, as for example shown in FIG. 3 of Example 2 below.

Preferably, the administration of the one or more HPV antigens and the recall antigen is performed by administering a composition comprising both the one or more HPV antigens and the recall antigen. But it can also be done by sequential separate administration of the one or more HPV antigens and the recall antigen, for instance by intradermal injection of the one or more HPV antigens in one composition and separate intradermal injection into the same spot of the recall antigen in a second composition.

Thus, in one embodiment, the composition comprising one or more HPV antigens also comprises the recall antigen.

In one embodiment, the steps of administering to the individual one or more HPV antigens and administering to the individual the recall antigen comprise intradermally injecting the one or more HPV antigens and the recall antigen. In other specific embodiments, the recall antigen and the HPV antigens are administered by subcutaneous injection. Intradermal injection is particularly preferred because Langerhans cells are the most common antigen presenting cells and are found in the greatest abundance in the skin.

In a specific embodiment, the one or more HPV antigens comprise an HPV E7 antigen.

In specific embodiments, the one or more HPV antigens are peptides of 8-100 amino acids in length, 8-70 amino acids in length, 8-50 amino acids in length, or 8-40 amino acids in length. In a more specific embodiment, the one or more peptides are chemically synthesized.

In a Phase I clinical trial of patients with biopsy-proven high-grade squamous intraepithelial (HSIL), women were treated with intradermal injection of a composition comprising HPV protein E6 residues 1-45 (SEQ ID NO:2), E6 46-80 (SEQ ID NO:3), E6 81-115 (SEQ ID NO:4), and E6 116-158 (SEQ ID NO:5), all mixed with CANDIN as an adjuvant. The dosages tested were 50 ug, 100 ug, and 250 ug of each of the peptides. It was surprisingly found that 4 of 6 subjects (67% in the 50 ug dose group, in 3 of 6 subjects (50%) in the 100 ug does group, and in 0 of 3 subjects in the 250 ug dose group had complete regression of their lesions. In addition, one additional subject in the 50 ug dose group had a partial regression (<0.2 mm2 lesion remaining). This is a surprising result that the lowest dose was the most effective. This is reported in Example 3 below.

Thus, another embodiment provides a unit dosage pharmaceutical composition comprising: 25 to 110 ug of a peptide consisting of SEQ ID NO:2, 25 to 110 ug of a peptide consisting of SEQ ID NO:3, 25 to 110 ug of a peptide consisting of SEQ ID NO:4, 25 to 110 ug of a peptide consisting of SEQ ID NO:5; and a recall antigen; in a unit dosage form for intradermal injection in a volume of 100 to 900 ul, 200 to 900 ul, 300 to 900 ul, or 100 to 600 ul.

The recall antigen should be in an amount and concentration sufficient to produce an induration response upon intradermal injection into a human—that is into a majority of immunocompetent adults who have previously been exposed to the antigen.

In a specific embodiment, the recall antigens is Candida extract.

In a specific embodiment, the unit dosage pharmaceutical composition comprises 200-400 ul of CANDIN or equivalent total potency of a Candida extract.

In a specific embodiment of the unit dosage pharmaceutical composition, the total volume is 200 to 500 ul.

In specific embodiments, the unit dosage pharmaceutical composition comprises 30 to 70 ug of each of the peptides, or in other embodiments about 50 ug of each of the peptides.

In specific embodiments, each of the peptides is acetylated at its amino terminus and amidated at its carboxy terminus.

In Example 3, the injecting the composition with 100 ug of each of the 4 peptides also worked well in causing regression of lesions. Thus, another embodiment provides a unit dosage pharmaceutical composition comprising: 55 to 150 ug of a peptide consisting of SEQ ID NO:2, 55 to 150 ug of a peptide consisting of SEQ ID NO:3, 55 to 150 ug of a peptide consisting of SEQ ID NO:4, 55 to 150 ug of a peptide consisting of SEQ ID NO:5; and a recall antigen; in a unit dosage form for intradermal injection in a volume of 100 to 900 ul.

Another embodiment provides a unit dosage pharmaceutical composition comprising: about 100 ug of a peptide consisting of SEQ ID NO:2, about 100 ug of a peptide consisting of SEQ ID NO:3, about 100 ug of a peptide consisting of SEQ ID NO:4, about 100 ug of a peptide consisting of SEQ ID NO:5; and a recall antigen; in a unit dosage form for intradermal injection in a volume of 100 to 900 ul.

Another embodiment provides a method of treating HPV infection comprising: administering to a patient intradermally a unit dosage pharmaceutical composition comprising: 25 to 110 ug of a peptide consisting of SEQ ID NO:2, 25 to 110 ug of a peptide consisting of SEQ ID NO:3, 25 to 110 ug of a peptide consisting of SEQ ID NO:4, 25 to 110 ug of a peptide consisting of SEQ ID NO:5; and a recall antigen; in a unit dosage form for intradermal injection in a volume of 100 to 900 ul.

In specific embodiments, the methods comprise injecting the patient intradermally with the unit dosage pharmaceutical composition on at least three successive occasions with no less than 5 days and no more than 28 days between each injection.

In another embodiment, the method comprises injecting the patient intradermally with the unit dosage pharmaceutical composition on at least three successive occasions with no less than 10 days and no more than 21 days between each injection.

In a specific embodiment, the method comprises injecting the patient intradermally with the unit dosage pharmaceutical composition on at least two successive occasions with no less than 10 days and no more than 21 days between each injection.

In a specific embodiment, the method comprises injecting the patient intradermally with the unit dosage pharmaceutical composition on at least three and no more than 6 occasions within a 2 year period with no less than 5 days and no more than 28 days between each injection.

It is shown herein in Example 2 that recall antigens, such as CANDIN, enhance the T cell immune response to the HPV peptides tested. A combination of a recall antigen and HPV peptides was contacted with peripheral blood mononuclear cells. Thus, administering a vaccine that includes a recall antigen together with disease-specific antigens may have general applicability to promote a cellular (T cell) immune response to the disease-specific antigens.

Thus, one embodiment provides a method of treating a disease caused by microorganism in a mammalian subject comprising: administering to the subject a composition comprising one or more antigens of the microorganism and administering to the subject a recall antigen that is not an antigen of the microorganism; wherein the recall antigen is administered to be in contact with the one or more antigens of the microorganism in the subject.

In specific embodiments, the microorganism may be a virus, bacteria, or fungus (for example, a yeast). In specific embodiments, the microorganism is not HPV. In specific embodiments, the microorganism is not herpes simplex virus.

The one or more antigens of the microorganism may be peptides in specific embodiments of 10-100, 8-100, 8-75, 8-50, 8-40, 10-75, 10-50, 10-40, 20-100, 20-75, 20-50, 20-40, 30-100, 30-75, 30-50, or 30-40 amino acid residues in length.

The peptides are preferably chemically synthesized, but they may also be produced in a recombinant organism from recombinant DNA technology. They may also be produced by other means known to persons of skill in the art, for instance by proteolysis of proteins of the microorganisms.

The peptides in some embodiments are acetylated at their amino termini or amidated at their carboxy termini, or both. In other embodiments, neither terminus is modified.

Preferably in the method the composition is administered by intradermal injection. But it may be administered by any suitable method, for instance by intramuscular injection.

One embodiment provides a method of stimulating a systemic T helper cell type 1 response in a mammal (preferably a human) in need thereof, the method comprising: injecting a composition comprising a recall antigen intradermally in a person in need thereof; wherein the method is not a method of treating a herpes simplex virus infection; and wherein the method does not comprise injecting a composition comprising a recall antigen intradermally into a viral epithelial lesion wherein the method increases T helper cell type 1 response in the mammal; and (i) wherein the mammal is infected with a microorganism and afflicted with a disease caused by the microorganism, and the composition comprising a recall antigen does not comprise an antigen of the microorganism infecting the person, or (ii) wherein the mammal is afflicted with a cancer or was afflicted with a cancer and the cancer is now in remission, and the composition comprising a recall antigen does not comprise an antigen of the cancer currently or previously afflicting the mammal.

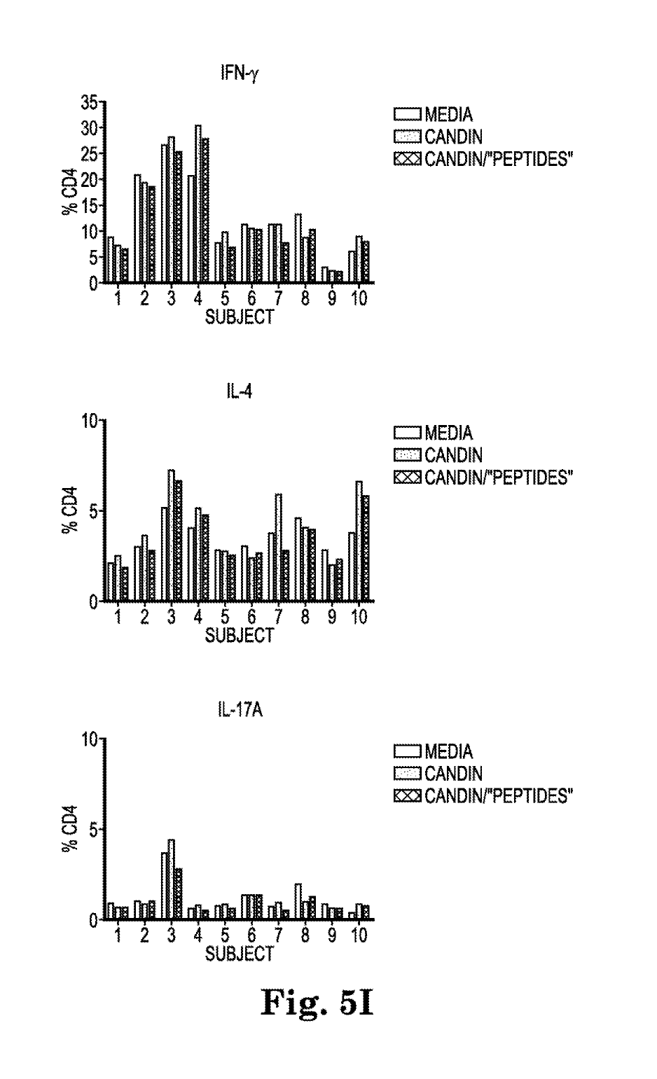

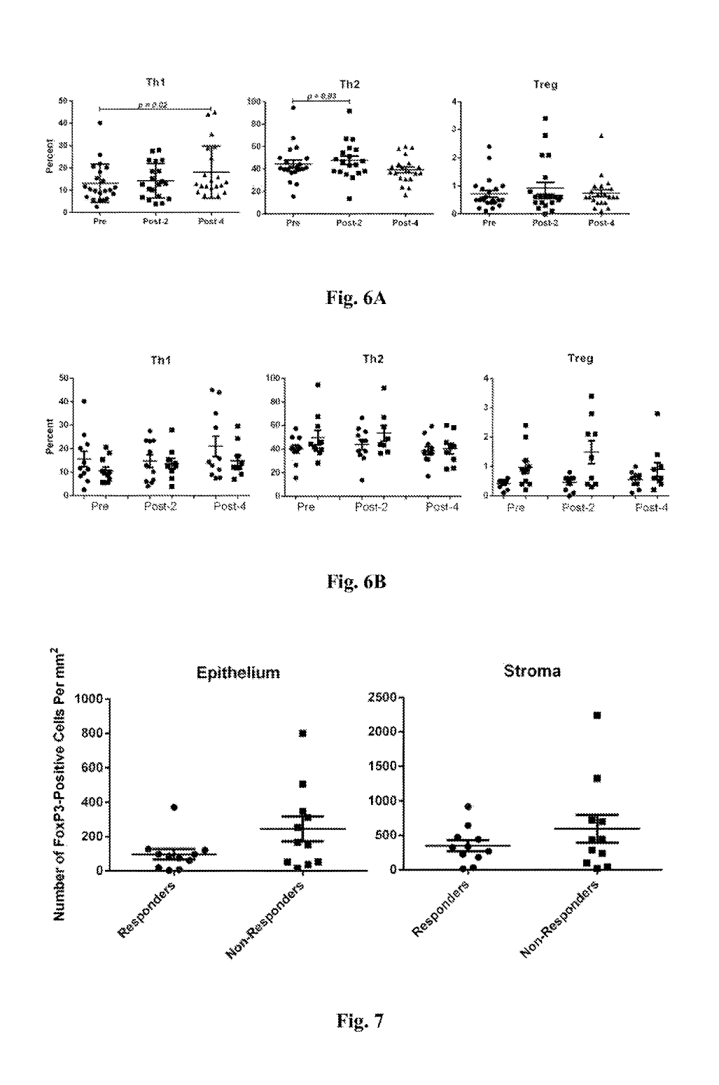

T helper cell type 1 response is assayed by the percentage of CD4 T cells that are CD4 T helper type 1 cells (Th1). CD4 T cells are defined as cells that are CD4+, positive for the CD4 marker. The Th1 subpopulation are the cells that are positive for CD4 and positive for Tbet (also known as T-bet). So “T helper cell type 1 response” is defined as the percentage of CD4+ cells that are Tbet+. The assay for this is a flow cytometry assay as described in Example 3 below with results shown in FIG. 6A and FIG. 6B.

In specific embodiments, the recall antigen is candida extract, mumps antigen, or trichophyton extract.

In specific embodiments of the method, the recall antigen stimulates IL-12 secretion from Langerhans cells in vitro.

In specific embodiments, the method of stimulating a systemic T helper cell type 1 response comprises injecting the recall antigen intradermally in the person at a dose level and on a dose schedule, wherein the recall antigen increases Th1 cells in most persons receiving intradermal injection of the recall antigen at the dose level and dose schedule.

In specific embodiments of the method of stimulating a systemic T helper cell type 1 response in a person in need thereof, the person is infected with HPV and afflicted with a disease caused by HPV, e.g., cervical cancer, head and neck cancer, warts, or high-grade squamous intraepithelial lesions.

Cancers caused by HPV include cervical cancer, head and neck cancer, vulvar cancer, anal cancer, vaginal cancer, and penile cancer. So where reference is made to “a cancer caused by HPV,” cervical cancer, head and neck cancer, vulvar cancer, anal cancer, vaginal cancer, and penile cancer are contemplated.

In specific embodiments of the method of stimulating a systemic T helper cells type 1 response, the method may further include administering an immunological checkpoint inhibitor to the person.

The immune system depends on multiple checkpoints or “immunological brakes” to avoid overactivation of the immune system against healthy cells, since that would be autoimmune disease. However, the activity of these checkpoints can be undesirable when more immune activity is wanted, such as in fighting cancer or a microbial infection. Tumor cells often take advantage of these checkpoints to escape detection by the immune system. The checkpoints are receptors on tumors or on immune cells, particularly T cells, interacting with tumors, where the interactions of the two receptors inhibits immune attack against the tumors.

One checkpoint is programmed death-ligand 1 (PDL1), which is overexpressed on some tumors. Another checkpoint is programmed cell death protein 1, also known as PD-1, which is a cell surface receptor on T cells that is a ligand to PDL1 and other proteins, and when it binds its ligands it down regulates immune activity. Another checkpoint is cytotoxic T-lymphocyte-associated protein 4 (CTLA-4), which is another cell surface receptor on T cells that when it binds its ligands down regulates immune activity. Examples of checkpoint inhibitors that have been shown to have efficacy in reducing tumor growth are antibodies against CTLA-4, PD-1, and PDL1. These immunological checkpoint inhibitors boost immune activity.

The term “immunological checkpoint inhibitor” as used herein refers to a compound that binds specifically to, and inhibits the activity of, a cell surface receptor that is present either on tumor cells or on T cells and that if present on tumor cells binds to T cells, where the cell surface receptor is involved in suppressing immune activity, where the “checkpoint inhibitor” by inhibiting the activity of the cell surface receptor increases immune activity.

By increasing Th1 activity and number, the intradermal injection of recall antigens also boosts immune activity by a different mechanism than immunological checkpoint inhibitors do. Thus, the two mechanisms may be synergistic.

In specific embodiments of stimulating a systemic T helper cells type 1 response, the method further comprises administering an anti-PD-1 antibody or an anti CTLA-4 antibody or an anti-PDL1 antibody to the person. In specific embodiments, the person is afflicted with cancer and the method further comprises administering an anti-PD-1 antibody or an anti-CTLA-4 antibody or an anti-PDL1 antibody to the person.

One embodiment provides a method of treating a microbial infection or cancer in a mammal (which may be a human) comprising: injecting a composition comprising a recall antigen intradermally in a mammal in need thereof; wherein the method is not a method of treating a herpes simplex virus infection; and wherein the method does not comprise injecting a composition comprising a recall antigen intradermally into a viral epithelial lesion; and (i) wherein the mammal is infected with a microorganism and afflicted with a disease caused by the microorganism, and the composition comprising a recall antigen does not comprise an antigen of the microorganism infecting the person; or (ii) wherein the mammal is afflicted with a cancer or was afflicted with a cancer and the cancer is now in remission, and the composition comprising a recall antigen does not comprise an antigen of the cancer currently or previously afflicting the mammal.

In specific embodiments, the recall antigen is candida extract, mumps antigen, or trichophyton extract.

In specific embodiments, the recall antigen stimulates IL-12 secretion from Langerhans cells in vitro.

In specific embodiments of the method the method increases percentage of CD4+ T cell that are Th1 cells in the person. CD4+ T cells are defined as cells that are CD4+, positive for the CD4 marker. The Th1 subpopulation are the cells that are positive for CD4 and positive for Tbet (also known as T-bet).

In specific embodiments, the method comprises injecting the recall antigen intradermally in the person at a dose level and on a dose schedule, wherein the recall antigen increases Th1 cells in most persons receiving intradermal injection of the recall antigen at the dose level and dose schedule.

In specific embodiments, the method is a method of treating HPV infection in a person in need thereof.

In specific embodiments, the person is afflicted with a cancer caused by HPV infection.

In specific embodiments, the method is a method of treating a viral infection. In other embodiments, the method is a method of treating a bacterial infection or a fungal infection. (The term “fungus” includes yeast herein, so the fungal infection may be a yeast infection.).

One embodiment of the invention provides a method of preventing cancer in a mammal comprising: injecting a composition comprising a recall antigen intradermally in the mammal. In more specific embodiments, the composition does not comprise an antigen of cancer or an HPV antigen.

In specific embodiments the mammal is a human.

The recall antigen in specific embodiments is Candida extract, mumps antigen, or Trichophyton extract.

In specific embodiments, the recall antigen stimulates IL-12 secretion from Langerhans cells in vitro.

In specific embodiments, the mammal is a human and the method comprises injecting the recall antigen intradermally in the human at a dose level and on a dose schedule, wherein the recall antigen increases Th1 cells in most humans receiving intradermal injection of the recall antigen at the dose level and dose schedule.

In specific embodiments, the cancer is a cancer caused by HPV infection.

In specific embodiments, the cancer is cervical cancer or head and neck cancer.

Another embodiment provides a method of stimulating a systemic T helper cell type 1 response in a mammal in need thereof, the method comprising: injecting a composition comprising a recall antigen intradermally in a mammal in need thereof; wherein the method is not a method of treating a herpes simplex virus infection; and wherein the method does not comprise injecting a composition comprising a recall antigen intradermally into a viral epithelial lesion; wherein the method increases T helper cell type 1 response in the mammal; and wherein the mammal was afflicted with a cervical cancer or head and neck cancer or a cancer caused by HPV and the cancer is now in remission.

In a more specific embodiment the composition further comprises HPV E6 protein or a plurality of peptide fragments of HPV E6 protein of 10-100 amino acid residues in length, the fragments collectively comprising at least 50% of SEQ ID NO:1.

In one embodiment, the composition comprises peptides consisting of residues 1-45, 46-80, 81-115, and 116-158 of SEQ ID NO:1.

In one embodiment, the composition comprises peptide fragments of HPV E6 and the peptides are acetylated or their amino termini or amidated on their carboxy termini, or acetylated on their amino termini and amidated on their carboxy termini.

Another embodiment provides a method of preventing growth of tumors or recurrence of cancer in a mammal comprising: injecting a composition comprising a recall antigen intradermally in a mammal in need thereof; wherein the method is not a method of treating a herpes simplex virus infection; and wherein the method does not comprise injecting a composition comprising a recall antigen intradermally into a viral epithelial lesion; wherein the method increases T helper cell type 1 response in the mammal; and wherein the mammal is afflicted with cervical cancer or head and neck cancer or a cancer caused by HPV, or the mammal was afflicted with cervical cancer or head and neck cancer or a cancer caused by HPV and the cancer is now in remission.

In a more specific embodiment, the composition further comprises HPV E6 protein or a plurality of peptide fragments of HPV E6 protein of 10-100 amino acid residues in length, the fragments collectively comprising at least 50% of SEQ ID NO:1.

In a more specific embodiment, the composition comprises peptides consisting of residues 1-45, 46-80, 81-115, and 116-158 of SEQ ID NO:1.

In one embodiment, the composition comprises peptide fragments of HPV E6 and the peptides are acetylated on their amino termini or amidated on their carboxy termini, or acetylated on their amino termini and amidated on their carboxy termini.

In a specific embodiment of the method the mammal was afflicted with cervical cancer or head and neck cancer or a cancer caused by HPV, and the cancer is now in remission, and the method is a method of preventing recurrence of the cancer.

### EXAMPLES

### Example 1. Solubilizing Amidated and Acetylated HPV E6 81-115 Peptide, and Formation of Pharmaceutical Composition

We attempted to make a pharmaceutical formulation with four HPV E6 peptides. The 4 peptides were peptides consisting of residues 1-45, 46-80, 81-115, and 116-158 of SEQ ID NO:1. Each of the peptides was amidated at its carboxyl terminus and acetylated at its amino terminus. The peptides were each chemically synthesized.

The HPV 16 E6 81-115 peptide was found to be insoluble in any suitable buffer for manufacturing. However, it was found that it could be solubilized and will stay soluble when added to 10 mM glutamate, pH 4.0 solution which already contains solubilized E6 1-45, E6 46-80, and E6 116-158 at 6 mg/ml concentration for each of the four peptides.

For the pharmaceutical formulation, this was mixed with trehalose as a stabilizing agent and glycine as tonicity modifier. The final concentrations of the formulation were 10 mM glutamate, 1.0% w/v trehalose, 2.0% w/v glycine, and 0.714 mg/ml each of the four peptides.

The formulation was lyophilized for storage, and reconstituted immediately before use by addition of the appropriate volume of water for injection to produce the concentrations stated above.

### Example 2: Candida Skin Test Reagent as a Novel Adjuvant for a Human Papilloma Virus Peptide-Based Therapeutic Vaccine

A vaccine adjuvant that can effectively promote cell-mediated immunity is currently not available. Because of the ability of a Candida skin test reagent injection to induce common wart regression, our group is using it as a novel adjuvant in a clinical trial of a peptide-based human papillomavirus therapeutic vaccine. The goal of this current study was to investigate the mechanisms of how Candida enhances the vaccine immune responses. Maturation effects on Langerhans cells, capacity to proliferate T-cells, expression of cytokines and pattern recognition receptors by Langerhans cells, and ability to induce Th1, Th2, and Th17 responses were investigated in healthy subjects. The vaccine, human papillomavirus peptides with Candida, demonstrated partial maturation effects on Langerhans cells indicated by significantly up-regulated CD40 (p=0.00007) and CD80 (p<0.00001) levels, and showed T-cell proliferative capacity (p<0.00001) when presented by Langerhans cells in vitro. Interestingly, the maturation effects were due to the peptides while Candida was responsible for the T-cell proliferation. The cytokine profile (IL-1β, IL-6, IL-8, IL-10, IL-12p40, IL-23Ap19, IFN-γ, and TNF-α) of Langerhans cells treated with the vaccine or Candida alone showed that IL-12p40 mRNA was most frequently induced, and IL-12p70 protein was detected in the supernatants. The presence of pattern recognition receptors known to associate with Candida albicans (DC-SIGN, dectin-1, dectin-2, galectin-3, mincle, mannose receptor, Toll-like receptors-1, 2, 4, 6, and 9) were demonstrated in all subjects. On the other hand, the induction of Th1 response demonstrated by IFN-γ secretion by CD4 cells stimulated with the vaccine or Candida pulsed Langerhans cells was demonstrated only in one subject. In summary, the Langerhans cell maturation effects of the vaccine were due to the peptides while the T-cell proliferative capacity was derived from Candida, and the most frequently induced cytokine was IL-12.

### ABBREVIATIONS

APCs, antigen presenting cells; HPV, human papillomavirus; LCs, Langerhans cells; MFI, mean fluorescence intensity; PAMPs, pathogen-associated molecular patterns; PBMC, peripheral blood mononuclear cells; PE, phycoerythrin; qRT-PCR, quantitative real-time PCR; PRRs, pattern recognition receptors.

### 1. Introduction

The most widely used adjuvant in approved human vaccines is an alum-based adjuvant that has been shown to elicit a predominantly Th2 immune response[1]. Therefore, the alum-based adjuvant would be useful in a vaccine designed to boost antibody responses, but not for a vaccine designed to stimulate cellular immune responses. Since successful clearance of human papillomavirus (HPV) infection is believed to be induced by cell-mediated immunity[2, 3], an adjuvant that would promote such an immunity is necessary, but not available.

Our group and others have shown that serial intra-lesional injections of common warts with skin testing reagents such as Candida, mumps, and/or Trichophyton can induce regression not only of treated warts but also of distant untreated warts[4-9]. In a Phase I clinical trial (NCT00569231), our group used intralesional injection of CANDIN (Allermed, San Diego, Calif.), a colorless extract of Candida albicans, to treat common warts. Resolution of treated warts occurred in 82% of the subjects, and anti-HPV T-cell responses were demonstrated[8]. Given that CANDIN is derived from C. albicans, it should contain numerous pathogen-associated molecular patterns (PAMPs). We hypothesized that CANDIN would be an effective vaccine adjuvant which would stimulate multiple pattern recognition receptors (PRRs) and induce innate as well as adaptive immunity.

Cervical cancer is almost always caused by high-risk HPV infection, and is the 2^{nd} most common cancer among women in the world. Two very effective prophylactic HPV vaccines, Gardasil® (Merck, NJ, USA) and Cervarix® (GlaxoSmithKline, Middlesex, UK), are available, and they work by inducing high titers of neutralizing antibody[10-12]. However, they are not effective for women with pre-existing HPV infection[10, 12, 13]. Therefore, a therapeutic HPV vaccine that can be used for those already infected with HPV and/or have developed HPV-associated neoplasia is not available. Our group studied naturally induced immunity in women with HPV infection and/or cervical lesions, and have found that the ability to induce T-cell responses against E6, one of the oncoproteins of high-risk HPVs, is associated with HPV clearance and regression of cervical lesions[3, 14, 15]. Therefore, we designed an HPV therapeutic vaccine which consists of four HPV type 16 E6 peptides and CANDIN, and are conducting a Phase I clinical trial (NCT01653249).

In the current study, we examined the immune enhancing effects of CANDIN as a vaccine adjuvant. Surprisingly, the E6 peptides were responsible for the partial maturation of Langerhans cells (LCs) while CANDIN was responsible for the T-cell proliferative effects. The most commonly induced cytokine by the LCs was IL-12.

### 2. Materials and Methods

### 2.0 Preparation of Primers.

A mixture of the HPV 16 E6 peptides was prepared and solubilized as described in Example 1.

### 2.1 Generation of Monocytes-Derived LCs

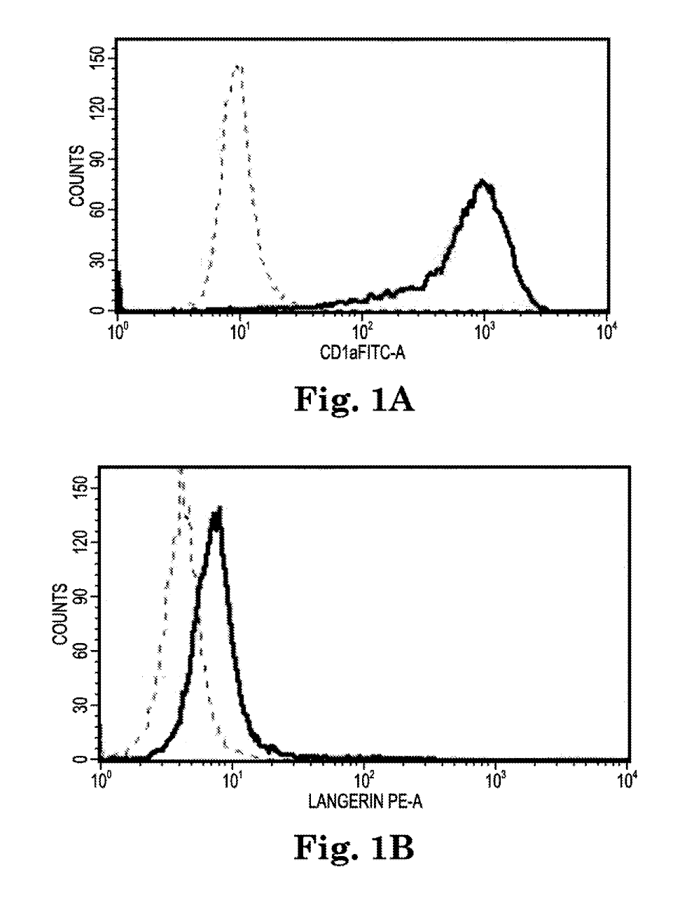

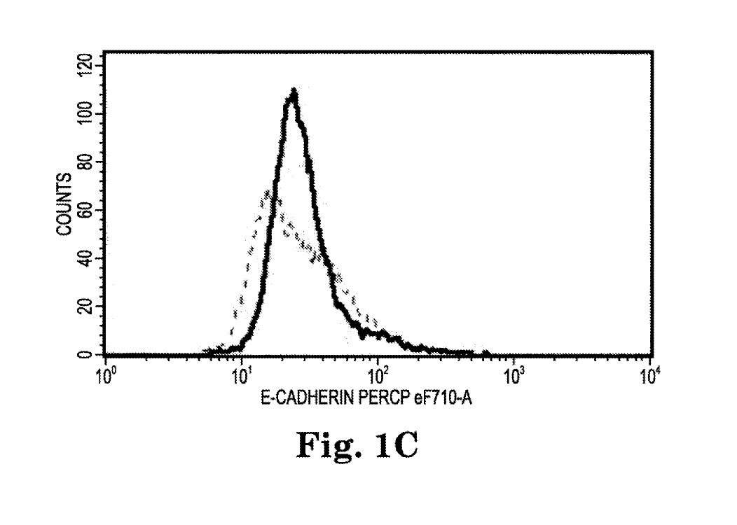

Mononuclear cells were collected from healthy blood donors (n=10) by apheresis (Key Biologics, LLC, Memphis, Tenn.). The subjects were numbered in a chronological order. Peripheral blood mononuclear cells (PBMCs) were purified using the ficoll gradient centrifugation method. Monocytes were negatively isolated from PBMC using Monocyte Isolation Kit II (Miltenyi Biotec, Auburn, Calif.), and were converted to LCs using granulocyte-macrophage colony-stimulating factor, IL-4, and transforming growth factor β-1 as described by Fahey et al.[17]. The effectiveness of conversion to LCs was demonstrated by detecting CD1a (eBioscience, San Diego, Calif.), Langerin (Beckman-Coulter, Brea, Calif.), and E-cadherin (eBioscience) using FACS Fortessa (University of Arkansas for Medical Sciences Microbiology and Immunology Flow Cytometry Core Laboratory) and CellQuest Pro software (BD Biosciences, San Jose, Calif.) in selected experiments (FIG. 1). Sufficient number of cells were available from all subjects except for subject 1 in whom the LC maturation experiment could not be performed.

2.2 Maturation Analysis of LCs Treated with CANDIN and/or HPV Peptides

CANDIN was dialyzed before use to remove a small amount of solvent (0.4% phenol) using Slide-A-Lyzer G2 Dialysis Cassette (Thermo Scientific, Rockford, Ill.). LCs were prepared as described above, and one million LCs each were treated with CANDIN (150 μl/ml), four current good manufacturing practice-grade HPV16 E6 peptides [E6 1-45, E6 46-80, E6 81-115, and E6 116-158 (referred to as “peptides” hereafter); 10 μg/ml/peptide; made by CPC Scientific, Sunnyvale, Calif. and vialed by Integrity Bio, Camarillo, Calif.], or CANDIN/“peptides”. Zymosan (10 μg/ml, InvivoGen, San Diego, Calif.), a yeast cell wall particle containing many polysaccharides including β-glucan and mannan[18], was used as a positive control. After 48 hour incubation, cells were stained with anti-human CD40 phycoerythrin (PE)-Cy5.5, CD80 fluorescein isothiocyanate, CD86 PE-Cy5 and HLA-DR PE (eBioscience, San Diego, Calif.). Ten thousand events were acquired, and the data were analyzed using Flowjo software (BD Biosciences).

2.3 Analysis of T Cell Proliferation Induced by LCs Treated with CANDIN and/or “Peptides”

On day 7 of LCs conversion, CD3 T cells from the same subjects were negatively isolated from PBMCs using Pan T-Cell Isolation Kit II (Miltenyi Biotec). To remove CD25 regulatory T cells, human CD25 Antibody-Biotin (Miltenyi Biotec) was added. T cell proliferation assay was performed in 6 replicate wells by co-culturing T cells (1.5×10⁶ cells/ml) with autologous LCs (3×10⁴ cells/ml) in 100 μl of complete Yssel's media (Gemini Bioproducts Inc, Woodland, Calif.) containing 1% human serum in each well of a 96-well plate. Wells containing cells only (T-cells and LCs), cells and CANDIN (150 μl/ml), cells and CANDIN/“peptides”, and cells and tetanus toxoid (500 ng/ml, EMD Millipore, Billerica, Mass.) were set up. After 7 days of incubation, 10 μl of alamarBlue (Life Technologies, Grand Island, N.Y.) was used to replace the corresponding volume of media in each well, then the plate was incubated at 37° C. for 6 hours. Fluorescence was measured (530 nm excitation wavelength and 590 nm emission wavelength) in media using BioTek Synergy-2 Multi Plate Reader (US BioTek, Seattle, Wash.).

2.4 Cytokine and PRR Analyses by Quantitative Real-Time PCR (qRT-PCR)

One million LCs each were treated with CANDIN (50 μl/ml, 100 μl, and 150 μl/ml) with or without “peptides” (10 μg/ml/peptide) at each CANDIN concentration. Zymosan was used as a positive control at 10 ug/ml and media only as a negative control. Cells were harvested for RNA after 8 and 24 hours. RNA was extracted using RNeasy kit (Qiagen, Valencia, Calif.), and treated with DNase I (Promega, Madison, Wis.). cDNA synthesis was carried out using SuperScript III first-strand synthesis system (Life Technologies).

Quantitative PCR analysis was performed in duplicate for cytokines including IL-1β, IL-6, IL-8, IL-10, IL-12p40, IL-23Ap19, IFN-γ and TNF-α using an iQ-SYBR mix (Bio-Rad, Hercules, Calif.). In addition, expressions of PRRs (DC-SIGN, dectin-1, dectin-2, galectin-3, mincle, mannose receptor, TLR-1, TLR-2, TLR-4, TLR-6, and TLR-9) known to associate with C. albicans[19-28] were examined. The primers used to detect IL-12 were previously reported by Vernal et al.[29]. All other primers were designed using Beacon Design software (Bio-Rad, Table 1). The threshold cycles were normalized to a human housekeeping gene, glyceraldehyde 3-phosphate dehydrogenase, and were calculated as fold change over untreated LCs at 8 hours. mRNA was considered to be detected when amplification of cDNA was demonstrated.

### 2.5 IL-12p70 Protein Analysis by ELISA

Supernatants from LCs treated with CANDIN (50 μl/ml, 100 μl/ml and 150 μl/ml) with or without “peptides” (10 μg/ml/peptide) from the qRT-PCR experiments at 24 hours were collected and tested using the IL-12p70 High Sensitivity ELISA kit (eBioscience). Values from media only wells were subtracted from experimental wells.

### 2.6 Intracellular Cytokine Staining

The methods were adapted according to those described by Zielinski et al.[30]. CD4 T-cells were negatively isolated from PBMCs using CD4 T Cell Isolation Kit II (Miltenyi Biotec) and were co-cultured with autologous LCs at a ratio of 50:1 (CCD4 T-cells:LCs). CANDIN (150 μl/ml) with or without “peptides” (10 μg/ml/peptide) were added to stimulate cells. Media alone was used as a negative control. After 6 days of co-culture, the cells were stimulated with phorbol 12-myristate 13-acetate (200 nM, Sigma, St. Louis, Mo.), and ionomycin (1 μg/ml, Sigma) for 2 hours. Then, Brefeldin A (10 μg/ml, eBioscience) was added for additional 2 hours. After being stained using fixable viability dye eFluor 450® (eBioscience), the cells were permeabilized/fixed and stained with anti-human IFN-γ PE, IL-17A peridinin chlorophyll protein-Cy5.5, IL-4 allophycocyanin, or relevant isotype controls (eBioscience). Ten thousand events were acquired using FACS Fortessa. Live lymphocytes were gated, and the percentages of IFN-γ, IL-17A and IL-4 positive CD4 T-cells were analyzed using FACS Diva (BD Biosciences) and Flowjo softwares.

### 2.7 Statistical Analysis

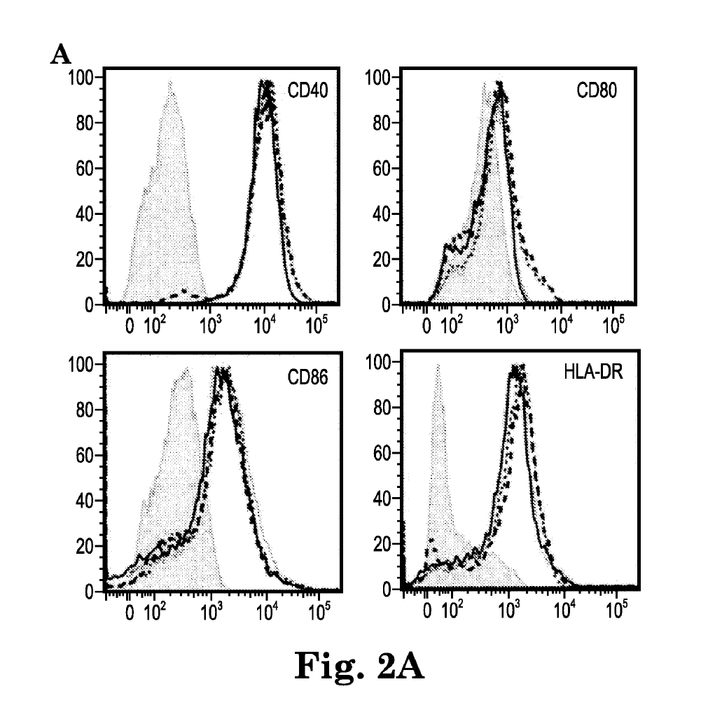

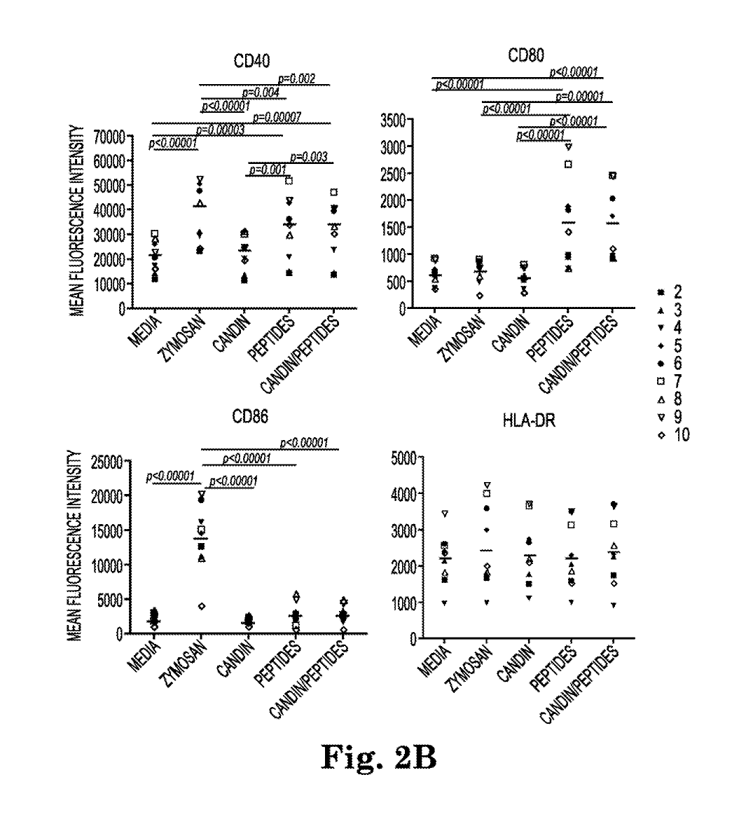

A mixed effects ANOVA was used to compare the groups while accounting for the dependence between groups. Tukey's multiple comparison procedure was used to perform all pairwise comparisons for maturation markers (FIG. 2B) while Dunnet's test was used to compare the media control values to the remaining groups for T-cell proliferation (FIG. 3).

### 3. Results

### 3.1 Phenotypic Maturation of LCs

We evaluated the maturation effects of CANDIN, and/or the E6 “peptides” on LCs (FIGS. 1-2). For CD40, statistically significant increases in mean fluorescence intensity (MFI) were observed with LCs treated with zymosan (p<0.00001), “peptides” (p=0.00003) and CANDIN/“peptides” (p=0.00007) compared to untreated LCs. In addition, MFIs of LCs treated with “peptides” and CANDIN/“peptides” were significantly higher than the MFI of LCs treated with CANDIN alone (p=0.001 and 0.003 respectively). For CD80, significant increases in MFIs were observed with LCs treated with “peptides” (p<0.00001) and CANDIN/“peptides” (p<0.00001) over media. Compared to CANDIN treated LCs, CD80 expression was significantly higher in “peptide” and CANDIN/“peptide” treated LCs (p<0.00001 for both). Only zymosan increased the MFI for CD86 significantly (p<0.00001). No significant increases were observed for HLA-DR. In summary, the “peptides” exerted partial LC maturation effects while CANDIN did not. Endotoxin levels for the “peptides” tested individually were all undetectable (<1.0 EU/mg).

3.2 T-Cell Proliferation Measured with alamarBlue

Proliferation was significantly increased with CANDIN (p<0.00001) and CANDIN/“peptides” (p<0.00001) over media (FIG. 3). “Peptides” did not induce measureable proliferation. Measurable proliferation with tetanus toxoid (increased fluorescence of ≧5000) was demonstrated in subjects 2 and 5, but overall no significant increase over media was observed (FIG. 3). Though unlikely, a possibility that LCs may have proliferated in addition to T-cells cannot be ruled out.

3.3 Expression of Cytokines by LCs Pulsed with CANDIN or CANDIN/“Peptides”

LCs from ten subjects were treated with CANDIN or CANDIN/“peptides”, and mRNA expression of 8 cytokines (Table 1) were examined by qRT-PCR (FIG. 4, Table 2). The amplifications of the intended products were confirmed by DNA sequencing after gel-purification from selected experiments. Overall, the cytokine expression profiles of LCs treated with CANDIN and CANDIN/“peptide” were similar. IL-12p40 was the most commonly enhanced cytokine (≧5 fold over untreated), and expression was detected in 5 subjects with CANDIN and in 7 subjects with CANDIN/“peptides”. IFN-γ was the 2^{nd} most commonly induced cytokine (6 subjects), and was detected in 5 subjects with CANDIN and in 4 subjects with CANDIN/“peptides”. IL-10 was also induced in 6 subjects: 4 subjects with CANDIN and 6 subjects with CANDIN/“peptide”. IL-6 and IL-23p19 were induced only with CANDIN (2 subjects for IL-6 and 1 subject for IL-23p19.) TNF-α was expressed only with CANDIN/“peptide” in 1 subject. IL-8 and IL-10 were not expressed in any subjects.

Supernatants from LCs treated with CANDIN or CANDIN/“peptides” for 24 hours were analyzed for the presence of IL12p70 protein. IL12p70 was detected in 27 of 30 samples treated with CANDIN (range 38 to 177 ng/ml) and in 27 of 30 samples treated with CANDIN/“peptides” (range 38 to 299 ng/ml).

TABLE 1
Primers used for qRT-PCR

| | Gene | | Forward primer | Reverse primer |
|---|---|---|---|---|
| Description | name | Accession no. | sequence | sequence |
| Interleukin 1 beta | hIL-1β | NM_000576.2 | CAG GGA CAG | CAC GCA GGA |
| | | | GAT ATG GAG | CAG GTA CAG |
| | | | CAA C | ATT C |
| Interleukin 6 | hIL-6 | NM_000600.3 | GTA GTG AGG | GGC ATT TGT |
| (interferon, beta | | | AAC AAG CCA | GGT TGG GTC |
| 2) | | | GAG C | AGG |
| Interleukin 8 | hIL-8 | NM_000584.3 | GAC CAC ACT | AAA CTT CTC |
| | | | GCG CCA ACA | CAC AAC CCT |
| | | | C | CTG C |
| Interleukin 10 | hIL-10 | NM_000572.2 | GGG TTG CCA | CGC CGT AGC |
| | | | AGC CTT GTC | CTC AGC CTG |
| | | | TG | |
| Interleukin 12B | hIL-12p40 | NM_002187.2 | CCC TGA CAT | AGG TCT TGT |
| | | | TCT GCG TTC A | CCG TGA AGA |
| | | | | CTC TA |
| Interleukin 23 | hIL23A | NM_016584.2 | AGT GTG GAG | GGG CTA TCA |
| alpha subunit p19 | p19 | | ATG GCT GTG | GGG AGC AGA |
| (IL23A) | | | ACC | GAA G |
| interferon, | hIFN-γ | NM_000619.2 | TGT GGA GAC | TGC TTT GCG |
| gamma | | | CAT CAA GGA | TTG GAC ATT |
| | | | AGA C | CAA G |
| Tumor Necrosis | hTNF-α | NM_000594.3 | GGG GTG GAG | ACG GCG ATG |
| Factor alpha | | | CTG AGA GAT | CGG CTG ATG |
| | | | AAC C | |
| DC-SIGN, CD | hDCSIGN | NM_001144899.1 | TGC AGT CTT | TGT TGG GCT |
| 209 | | | CCA GAA GTA | CTC CTC TGT |
| | | | ACC GCT | TCC AAT |
| C-type lectin | hDectin1 | NM_197947.2 | TGC TTG GTA | GGT TGA CTG |
| domain family 7, | | | ATA CTG GTG | TGG TTC TCT T |
| member A | | | ATA G | |
| (CLEC7A) | | | | |
| C-type lectin | hDectin2 | NM_001007033 | AAC ACA GAA | TCC AGA AGA |
| domain family 6, | | | GCA GAG CAG | CTA TTG AAG |
| member A | | | AAT | CAC ATT |
| (CLEC6A) | | | | |
| Lectin, | hGalectin3 | NM_001177388.1 | TGT GCC TTA | TTC TGT TTG |
| galactoside- | | | TAA CCT GCC | CAT TGG GCT |
| binding, soluble, | | | TTT GCC | TCA CCG |
| 3 (LGAL3) | | | | |
| C-type lectin | hMincle | NM_014358.2 | TCA GAA TAC | TGG TTA CAG |
| domain family 4, | | | CGG TGT GGC | CCT GTT TGG |
| member E | | | CTT TCT | AGC TGA |
| (CLEC4E) | | | | |
| Mannose | hMRC2 | NM_006039.4 | AGC AAC GTC | AGA ACT GTG |
| receptor, C type2 | | | ACC AAA GAA | CCT CTG ACC |
| | | | ACG CAG | ACT TCA |
| Toll-Like | hTLR1 or | NM_003263.3 or | ATG TGG CAG | TCT GGA AGA |
| Receptor 1/6* | TLR6 | NM_006068.4 | CTT TAG CAG | AAT CAG CCG |
| | | | CCT TTC | ATG GGT |
| Toll-Like | hTLR2 | NM_003264 | TGC TGC CAT | CAC TCC AGG |
| Receptor 2 | | | TCT CAT TCT | TAG GTC TTG |
| Toll-Like | hTLR4 | NM_138557 | CGT GCT GGT | GGT AAG TGT |
| Receptor 4 | | | ATC ATC TTC | TCC TGC TGA G |
| | | | AT | |
| Toll-Like | hTLR9 | NM_017442.3 | ATC TGC ACT | AAG GCC AGG |
| Receptor 9 | | | TCT TCC AAG | TAA TTG TCA |
| | | | GCC TGA | CGG AGA |
| Glyceraldehyde- | hGAPDH | NM_002046.4 | GGA CCT GAC | GTA GCC CAG |
| 3-phosphate | | | CTG CCG TCT | GAT GCC CTT GA |
| dehydrogenase | | | AG | |

*The same primers were used to analyze TLR 1 and 6 amplifying a 100% homologous region
between the two genes.

### 3.4 Expression of PRRs on LCs

All 11 PRRs examined were detectable in untreated LCs of all subjects (data not shown). Upon stimulation with CANDIN or CANDIN/“peptides”, few PRRs showed increased expression (≧5 fold over untreated). No obvious differences were observed in PRRs expressed between CANDIN- and CANDIN/“peptide”-treated LCs. The expression of TLR-9 was increased in 3 subjects (5 to 18 fold with CANDIN and 9 to 16 fold with CANDIN/“peptides”), mincle in 2 subjects (5 fold with CANDIN and CANDIN/“peptides”), mannose receptor in 2 subjects (5 to 9 fold with CANDIN and 5 to 11 fold with CANDIN/“peptides”), dectin-2 in 2 subjects (5 to 54 fold with CANDIN and 5 to 8 fold with CANDIN/“peptides”), and DC-SIGN in 1 subject (5 to 22 fold with CANDIN). In 5 subjects with increased expression of PRRs, 3 of them showed the increased expressions of two or more PRRs in LCs.

3.5 Intracellular Cytokine Expression of CD4 T-Cells Stimulated with CANDIN-Pulsed LCs or CANDIN/“Peptides”-Pulsed LCs

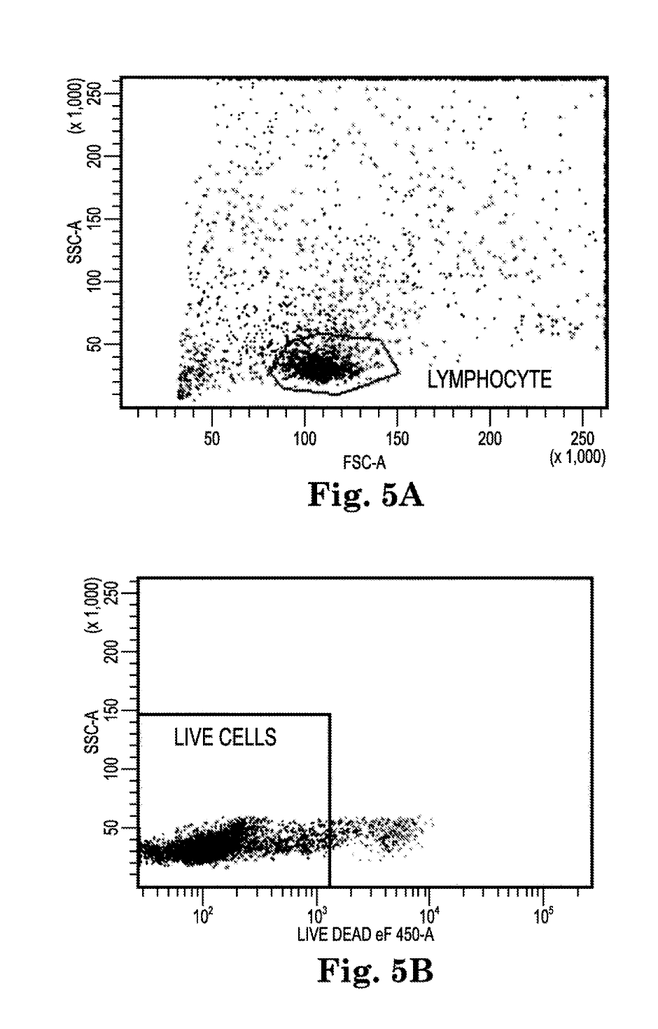

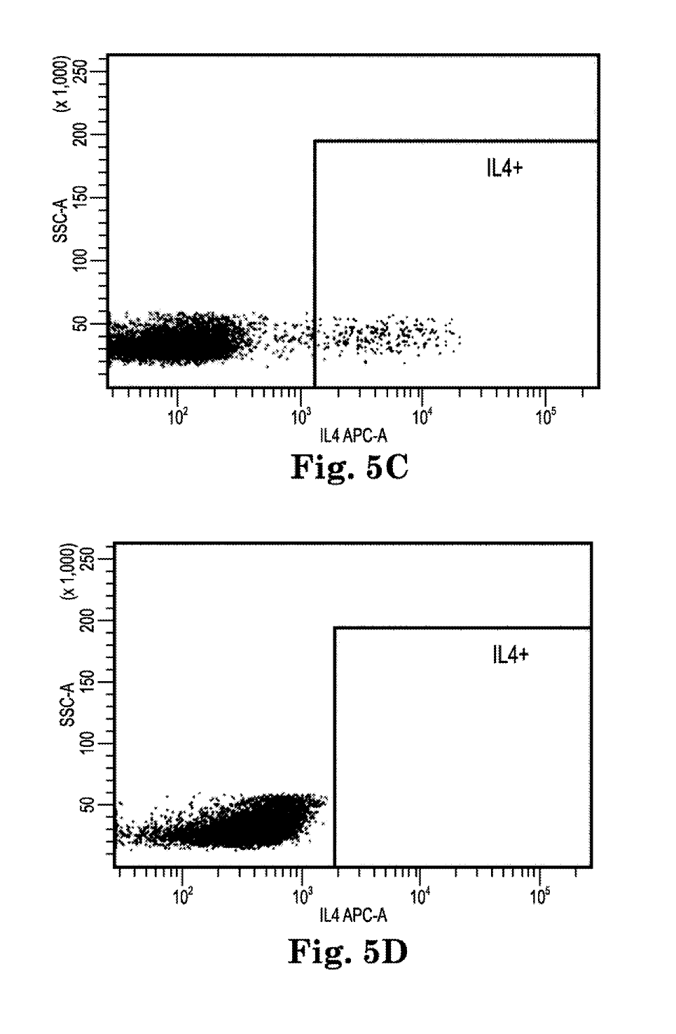

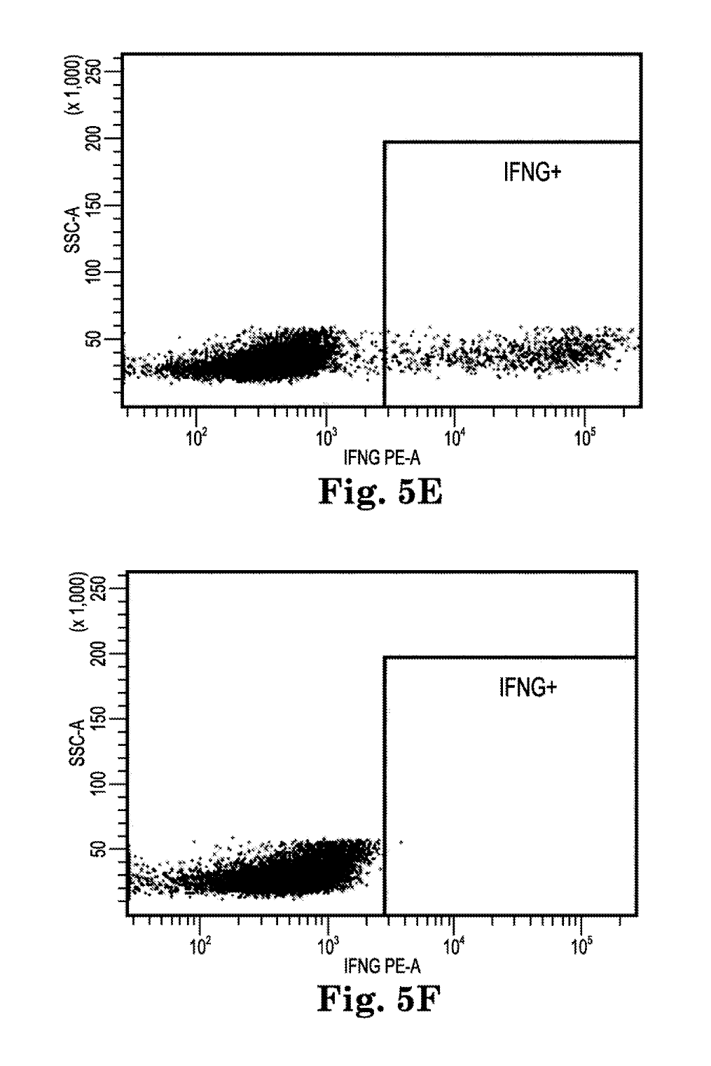

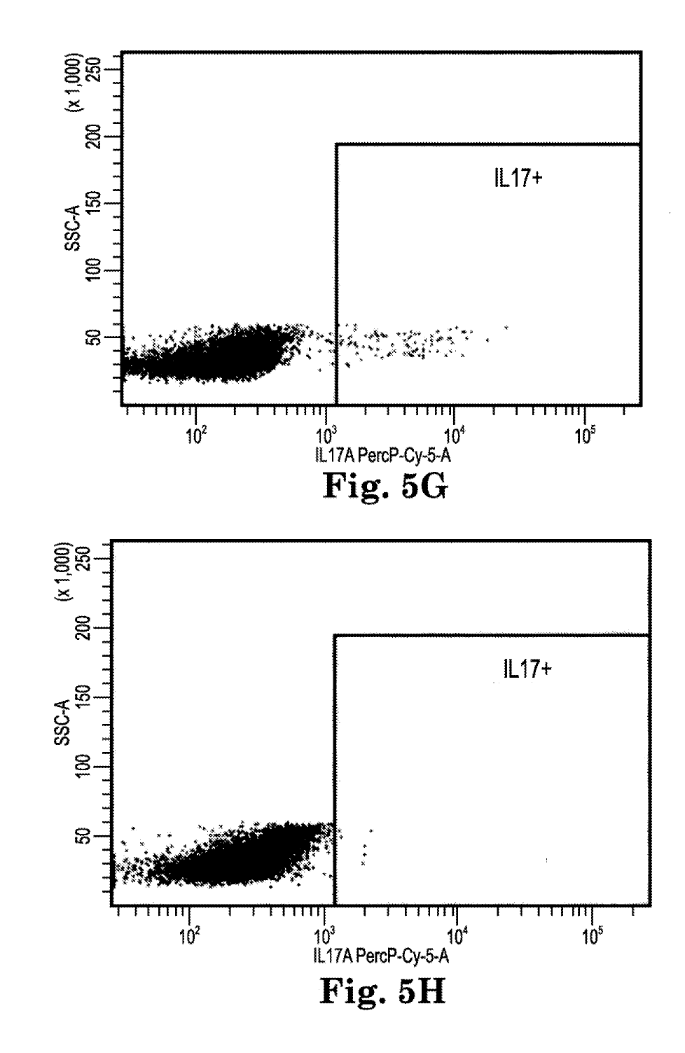

CD4 T-cells stimulated with CANDIN or CANDIN/“peptides”-treated LCs from ten subjects were stained for intracellular secretion of IFN-γ (Th1), IL-4 (Th2) and IL-17A (Th17) (FIG. 5). Increased IFN-γ secretions (>5%) were observed in CD4 T-cells exposed to CANDIN or CANDIN/“peptides”-treated LCs over media in subject 4 (9.5% and 6.9% respectively). Overall, no differences were seen in the secretion of IFN-γ, IL-4 and IL-17A between CD4 T-cells treated with LCs alone and LCs treated with CANDIN as well as between LCs alone and LCs treated with CANDIN/“peptides”.

### 4. Discussion

“Adjuvant” is derived from a Latin word, adjuvare, and means to help or to enhance. An effective vaccine adjuvant should be able to promote a strong immune response against the vaccine antigen in terms of size and durability. Antigen presenting cells (APCs) play a critical role in the initiation of immune responses. One of the desired features of an adjuvant is the ability to enhance maturation of APCs and the consequent priming of effective T-cell responses. CD40 and CD80 have been demonstrated to be critical for the activation of antigen-specific T-helper cells[31] and cytotoxic T-cells[32]. Our results have shown that the “peptides” can induce significantly higher expression of CD40 and CD80. This HPV therapeutic vaccine may be a rare vaccine in that the peptide antigens rather than the adjuvant are more able to mature APCs. These results are different from those reported by Romagnoli et al. who showed up-regulation of CD40, CD80, CD86 and HLA-DR on dendritic cells by C. albicans[33]. Since endotoxin was undetectable in “peptides”, it is unlikely that contamination may have contributed to the unexpected partial maturation effects on the LCs. We focused on examining maturation effects of LCs because our vaccine was formulated for intradermal route in order to take advantage of abundant LCs in epidermis. Studying maturation effects on other APCs such as dendritic cells and monocytes would be important in the future.

C. albicans as a component of the normal flora often colonizes the skin and the mucosal surfaces of healthy individuals. Underlying acquired immunity to C. albicans is usually present in immunocompetent individuals[34]. In this study, CANDIN and CANDIN/“peptides”, but not “peptides”, induced significant T-cell proliferation. Similar to our results, Gordon et al. demonstrated skin test positive reactions to C. albicans in 92% of healthy subjects[35], and Bauerle et al. demonstrated Candida-specific T-cell responses in 71% of healthy subjects. CANDIN is being used clinically to assess the intactness of cell-mediated immunity, so it is consistent with that that we find here that an extract from C. albicans has a T cell proliferative effect. Unfortunately, however, the maturation effects of C. albicans[33] are lost in the extract. On the other hand, it is found here that the “peptides” exert some maturation effects.

In creating this vaccine, an obstacle was encountered in being able to develop a formulation in which the “peptides” were soluble, as the E6 protein is known to be hydrophobic. While they remain soluble in acidic pH of the formulation, they are insoluble and form microparticles at a neutral pH (unpublished data). This unusual property may be contributing to the maturation effects by stimulating LCs to phagocytose these microparticles.

PRR signaling can induce APCs to express co-stimulatory molecules and cytokines necessary for activation and differentiation of T lymphocytes[37]. The cooperation of different PRRs in APCs by stimulating multiple PRRs leads to synergistic Th1[20, 38] and cytotoxic T-lymphocyte responses[39]. C. albicans has been shown to activate many PRRs including DC-SIGN[19], dectin-1[20], dectin-2[21], galectin-3[22], mannose receptor[19], mincle[40], and some TLRs[25-27, 41, 42]. Since some PRRs are increased during activation[43, 44], we investigated the presence and amplified expression of these PRRs. In this study, all PRRs examined were expressed by CANDIN and CANDIN/“peptide” pulsed LCs, and increased expressions of certain PRRs (DC-SIGN, dectin-2, mincle, monocyte receptor and TLR-9) were demonstrated in 5 of 10 subjects. Further investigations are necessary to determine which PRRs may have a role in transducing the signals from this HPV therapeutic vaccine. Dectin-1 in conjunction with TLR-2 can activate NF-κB[20], and dectin-1 can also independently mediate NFAT activation in dendritic cells leading to expression of inflammatory mediators such as IL-12p70[45]. Therefore, it would be interesting to investigate whether CANDIN or CANDIN/“peptide” has any role in NF-κB and NFAT activation in the future.

Cytokines secreted by APCs play important roles in the process of differentiation of T-helper cells into Th1, Th2, or Th17 cells. IL-12p70 directs Th1 response while IL-10 and IL-6 direct the Th17 response[37, 46]. The cytokine profile in treated LCs showed IL-12p40 was the most commonly enhanced cytokine and IL-12p70 was also detected at a protein level. Published studies showed that C. albicans can induce the differentiation of specific Th1 and Th17 cells[30, 33], and Candida-specific Th1 immune responses can be detected in healthy subjects[47, 48]. These data lead us to anticipate the extract of C. albicans, CANDIN, to induce a Th1 and Th17 skewing effect. Though an increased Th1 response (IFN-γ secretion >5%) was observed in one subject, the overall results from ten subjects showed no skewing towards Th1 and Th17 responses. It may be that Candida exerts Th1 and Th17 effects through multiple mechanisms. There exist other subsets of APCs in dermis, like dermal DCs[49], which may play roles in the process of antigen presentation and T-cell activation. Furthermore, it would be important to assess the ability of this HPV therapeutic vaccine to induce HPV-specific T-cell responses. This is being investigated in the context of the ongoing clinical trial.

In summary, “peptides” (antigens) are responsible for the LC maturation effects while CANDIN (adjuvant) induces significant T-cell proliferation for this HPV therapeutic vaccine. Therefore, the antigens and the adjuvant have complementary immune enhancing effects. With time, the ongoing clinical trial will reveal whether these complementing effects will translate into effective clinical responses.

### References for Example 2

[1] Gupta R K. Aluminum compounds as vaccine adjuvants. Adv Drug Deliv Rev. 1998; 32:155-72.
[2] Farhat S, Nakagawa M, Moscicki A B. Cell-mediated immune responses to human papillomavirus 16 E6 and E7 antigens as measured by interferon gamma enzyme-linked immunospot in women with cleared or persistent human papillomavirus infection. Int J Gynecol Cancer. 2009; 19:508-12.
[3] Nakagawa M, Gupta S K, Coleman H N, Sellers M A, Banken J A, Greenfield W W. A favorable clinical trend is associated with CD8 T-cell immune responses to the human papillomavirus type 16 e6 antigens in women being studied for abnormal pap smear results. J Low Genit Tract Dis. 2010; 14:124-9.
[4] Clifton M M, Johnson S M, Roberson P K, Kincannon J, Horn T D. Immunotherapy for recalcitrant warts in children using intralesional mumps or Candida antigens. Pediatr Dermatol. 2003; 20:268-71.
[5] Horn T D, Johnson S M, Helm R M, Roberson P K. Intralesional immunotherapy of warts with mumps, Candida, and Trichophyton skin test antigens: a single-blinded, randomized, and controlled trial. Arch Dermatol. 2005; 141:589-94.
[6] Johnson S M, Horn T D. Intralesional immunotherapy for warts using a combination of skin test antigens: a safe and effective therapy. J Drugs Dermatol. 2004; 3:263-5.
[7] Johnson S M, Roberson P K, Horn T D. Intralesional injection of mumps or Candida skin test antigens: a novel immunotherapy for warts. Arch Dermatol. 2001; 137:451-5.
[8] Kim K H, Horn T D, Pharis J, Kincannon J, Jones R, O'Bryan K, et al. Phase 1 clinical trial of intralesional injection of Candida antigen for the treatment of warts. Arch Dermatol. 2010; 146:1431-3.
[9] Phillips R C, Ruhl T S, Pfenninger J L, Garber M R. Treatment of warts with Candida antigen injection. Arch Dermatol. 2000; 136:1274-5.
[10] Quadrivalent vaccine against human papillomavirus to prevent high-grade cervical lesions. N Engl J Med. 2007; 356:1915-27.
[11] Harper D M. Currently approved prophylactic HPV vaccines. Expert Rev Vaccines. 2009; 8:1663-79.
[12] Schiller J T, Castellsague X, Villa L L, Hildesheim A. An update of prophylactic human papillomavirus L1 virus-like particle vaccine clinical trial results. Vaccine. 2008; 26 Suppl 10:K53-61.
[13] Hildesheim A, Herrero R, Wacholder S, Rodriguez AC, Solomon D, Bratti M C, et al. Effect of human papillomavirus 16/18 L1 viruslike particle vaccine among young women with preexisting infection: a randomized trial. JAMA. 2007; 298:743-53.
[14] Kim K H, Greenfield W W, Cannon M J, Coleman H N, Spencer H J, Nakagawa M. CD4+ T-cell response against human papillomavirus type 16 E6 protein is associated with a favorable clinical trend. Cancer Immunol Immunother. 2012; 61:63-70.
[15] Nakagawa M, Stites D P, Patel S, Farhat S, Scott M, Hills N K, et al. Persistence of human papillomavirus type 16 infection is associated with lack of cytotoxic T lymphocyte response to the E6 antigens. J Infect Dis. 2000; 182:595-8.
[16] Igyarto B Z, Kaplan D H. Antigen presentation by Langerhans cells. Curr Opin Immunol. 2013; 25:115-9.
[17] Fahey L M, Raff A B, Da Silva D M, Kast W M. Reversal of human papillomavirus-specific T cell immune suppression through TLR agonist treatment of Langerhans cells exposed to human papillomavirus type 16. J Immunol. 2009; 182:2919-28.
[18] Sato M, Sano H, Iwaki D, Kudo K, Konishi M, Takahashi H, et al. Direct binding of Toll-like receptor 2 to zymosan, and zymosan-induced NF-kappa B activation and TNF-alpha secretion are down-regulated by lung collectin surfactant protein A. J Immunol. 2003; 171:417-25.
[19] Cambi A, Netea M G, Mora-Montes H M, Gow N A, Hato S V, Lowman D W, et al. Dendritic cell interaction with Candida albicans critically depends on N-linked mannan. J Biol Chem. 2008; 283:20590-9.
[20] Gantner B N, Simmons R M, Canavera S J, Akira S, Underhill D M. Collaborative induction of inflammatory responses by dectin-1 and Toll-like receptor 2. J Exp Med. 2003; 197:1107-17.
[21] Sato K, Yang X L, Yudate T, Chung J S, Wu J, Luby-Phelps K, et al. Dectin-2 is a pattern recognition receptor for fungi that couples with the Fc receptor gamma chain to induce innate immune responses. J Biol Chem. 2006; 281:38854-66.
[22] Jouault T, El Abed-El Behi M, Martinez-Esparza M, Breuilh L, Trinel P A, Chamaillard M, et al. Specific recognition of Candida albicans by macrophages requires galectin-3 to discriminate Saccharomyces cerevisiae and needs association with TLR2 for signaling. J Immunol. 2006; 177:4679-87.
[23] Bugarcic A, Hitchens K, Beckhouse A G, Wells C A, Ashman R B, Blanchard H. Human and mouse macrophage-inducible C-type lectin (Mincle) bind Candida albicans. Glycobiology. 2008; 18:679-85.
[24] Netea M G, Gow N A, Munro C A, Bates S, Collins C, Ferwerda G, et al. Immune sensing of Candida albicans requires cooperative recognition of mannans and glucans by lectin and Toll-like receptors. J Clin Invest. 2006; 116:1642-50.
[25] Plantinga T S, Johnson M D, Scott W K, van de Vosse E, Velez Edwards D R, Smith P B, et al. Toll-like receptor 1 polymorphisms increase susceptibility to candidemia. J Infect Dis. 2012; 205:934-43.
[26] Villamon E, Gozalbo D, Roig P, O'Connor J E, Fradelizi D, Gil M L. Toll-like receptor-2 is essential in murine defenses against Candida albicans infections. Microbes Infect. 2004; 6:1-7.
[27] Netea M G, Van Der Graaf C A, Vonk A G, Verschueren I, Van Der Meer J W, Kullberg B J. The role of toll-like receptor (TLR) 2 and TLR4 in the host defense against disseminated candidiasis. J Infect Dis. 2002; 185:1483-9.
[28] Salvenmoser S, Seidler M J, Dalpke A, Muller F M. Effects of caspofungin, Candida albicans and Aspergillus fumigatus on toll-like receptor 9 of GM-CSF-stimulated PMNs. FEMS Immunol Med Microbiol. 2010; 60:74-7.
[29] Vernal R, Leon R, Silva A, van Winkelhoff A J, Garcia-Sanz J A, Sanz M. Differential cytokine expression by human dendritic cells in response to different Porphyromonas gingivalis capsular serotypes. J Clin Periodontol. 2009; 36:823-9.
[30] Zielinski C E, Mele F, Aschenbrenner D, Jarrossay D, Ronchi F, Gattorno M, et al. Pathogen-induced human TH17 cells produce IFN-gamma or IL-10 and are regulated by IL-1beta. Nature. 2012; 484:514-8.
[31] Schweitzer A N, Borriello F, Wong RC, Abbas A K, Sharpe A H. Role of costimulators in T cell differentiation: studies using antigen-presenting cells lacking expression of CD80 or CD86. J Immunol. 1997; 158:2713-22.
[32] Bennett S R, Carbone FR, Karamalis F, Flavell R A, Miller J F, Heath W R. Help for cytotoxic-T-cell responses is mediated by CD40 signalling. Nature. 1998; 393:478-80.
[33] Romagnoli G, Nisini R, Chiani P, Mariotti S, Teloni R, Cassone A, et al. The interaction of human dendritic cells with yeast and germ-tube forms of Candida albicans leads to efficient fungal processing, dendritic cell maturation, and acquisition of a Th1 response-promoting function. J Leukoc Biol. 2004; 75:117-26.
[34] Romani L. Innate and adaptive immunity in Candida albicans infections and saprophytism. J Leukoc Biol. 2000; 68:175-9.
[35] Gordon E H, Krouse H A, Kinney J L, Stiehm E R, Klaustermeyer W B. Delayed cutaneous hypersensitivity in normals: choice of antigens and comparison to in vitro assays of cell-mediated immunity. J Allergy Clin Immunol. 1983; 72:487-94.
[36] Bauerle M, Schroppel K, Taylor B, Bergmann S, Schmitt-Haendle M, Harrer T. Analysis of the Candida albicans-specific T-cell response and oropharyngeal Candida colonization in a cohort of HIV-1-infected patients. Eur J Med Res. 2006; 11:479-84.
[37] Iwasaki A, Medzhitov R. Regulation of adaptive immunity by the innate immune system. Science. 2010; 327:291-5.
[38] Napolitani G, Rinaldi A, Bertoni F, Sallusto F, Lanzavecchia A. Selected Toll-like receptor agonist combinations synergistically trigger a T helper type 1-polarizing program in dendritic cells. Nat Immunol. 2005; 6:769-76.
[39] Warger T, Osterloh P, Rechtsteiner G, Fassbender M, Heib V, Schmid B, et al. Synergistic activation of dendritic cells by combined Toll-like receptor ligation induces superior CTL responses in vivo. Blood. 2006; 108:544-50.
[40] Wells C A, Salvage-Jones J A, Li X, Hitchens K, Butcher S, Murray R Z, et al. The macrophage-inducible C-type lectin, mincle, is an essential component of the innate immune response to Candida albicans. J Immunol. 2008; 180:7404-13.
[41] Miyazato A, Nakamura K, Yamamoto N, Mora-Montes H M, Tanaka M, Abe Y, et al. Toll-like receptor 9-dependent activation of myeloid dendritic cells by Deoxynucleic acids from Candida albicans. Infect Immun. 2009; 77:3056-64.
[42] Netea M G, van de Veerdonk F, Verschueren I, van der Meer J W, Kullberg B J. Role of TLR1 and TLR6 in the host defense against disseminated candidiasis. FEMS Immunol Med Microbiol. 2008; 52:118-23.
[43] Biswas I, Garg I, Singh B, Khan G A. A key role of toll-like receptor 3 in tissue factor activation through extracellular signal regulated kinase 1/2 pathway in a murine hypoxia model. Blood Cells Mol Dis. 2012; 49:92-101.
[44] Sinha S, Guo Y, Thet S, Yuan D. IFN type I and type II independent enhancement of B cell TLR7 expression by natural killer cells. J Leukoc Biol. 2012; 92:713-22.
[45] Goodridge H S, Simmons R M, Underhill D M. Dectin-1 stimulation by Candida albicans yeast or zymosan triggers NFAT activation in macrophages and dendritic cells. J Immunol. 2007; 178:3107-15.
[46] Zhou L, Chong M M, Littman D R. Plasticity of CD4+ T cell lineage differentiation. Immunity. 2009; 30:646-55.
[47] La Sala A, Urbani F, Torosantucci A, Cassone A, Ausiello C M. Mannoproteins from Candida albicans elicit a Th-type-1 cytokine profile in human Candida specific long-term T cell cultures. J Biol Regul Homeost Agents. 1996; 10:8-12.
[48] Nisini R, Romagnoli G, Gomez M J, La Valle R, Torosantucci A, Mariotti S, et al. Antigenic properties and processing requirements of 65-kilodalton mannoprotein, a major antigen target of anti-Candida human T-cell response, as disclosed by specific human T-cell clones. Infect Immun. 2001; 69:3728-36.
[49] Valladeau J, Saeland S. Cutaneous dendritic cells. Semin Immunol. 2005; 17:273-83.

### Example 3

### A Phase I Dose-Escalation Clinical Trial of a Peptide-Based Human Papillomavirus Therapeutic Vaccine with Candida Skin Test Reagent as a Novel Vaccine Adjuvant for Treating Women with Biopsy-Proven Cervical Intraepithelial Neoplasia 2/3

### ABSTRACT

### Purpose

Non-surgical treatments for cervical intraepithelial neoplasia 2/3 (CIN2/3) are needed as surgical treatments have been shown to double preterm delivery rate. The goal of this study was to demonstrate safety of a human papillomavirus (HPV) therapeutic vaccine called PepCan, which consists of four current good manufacturing production-grade peptides covering the HPV type 16 E6 protein and Candida skin test reagent as a novel adjuvant.

### Patients and Methods

The study was a single-arm, single-institution, dose-escalation Phase I clinical trial, and the patients (n=24) were women with biopsy-proven CIN2/3. Four injections were administered intradermally every 3 weeks in limbs. Loop electrical excision procedure was performed 12 weeks after the last injection for treatment and histological analysis. Six subjects each were enrolled (50, 100, 250, and 500 ug per peptide).

### Results

The most common adverse events were injection site reactions, and none of the patients experienced dose-limiting toxicities. The best histological response was seen at the 50 ug dose level with a regression rate of 83% (n=6), and the overall rate was 52% (n=23). Vaccine-induced immune responses to E6 were detected in 65% of recipients (significantly in 43%). Systemic T-helper type 1 (Th1) cells were significantly increased after 4 vaccinations (p=0.02).

### Conclusion

This study demonstrated that PepCan is safe. A significantly increased systemic level of Th1 cells suggests that Candida, which induces interleukin-12 in vitro, may have a Th1 promoting effect. A Phase II clinical trial to assess the full effect of this vaccine is warranted.

### LIST OF ABBREVIATIONS AND ACRONYMS

AE, adverse event
CIN 2/3, cervical intraepithelial neoplasia 2/3
ELISPOT, enzyme-linked immunospot
HPV, human papillomavirus
IL-12, interleukin 12
LEEP, loop electrical excision procedure
PBMC, peripheral blood mononuclear cell
Th1, T-helper type 1
Th2, T-helper type 2
Treg, regulatory T-cell

### INTRODUCTION

Cervical intraepithelial neoplasia 2/3 (CIN2/3) is a precursor of cervical cancer which is the fourth most common cancer among women globally despite availabilities of effective screening tests and prophylactic vaccines. The annual global incidence of cervical cancer is 528,000 cases and the mortality is 266,000 cases.¹ It is almost always caused by human papillomavirus (HPV). HPV causes not only cervical cancer, but also anal, oropharyngeal, penile, vaginal, and vulvar cancers; it is estimated to be responsible for 5.2% of cancer cases in the world.^{2, 3}

Standard surgical treatments of CIN2/3 such as loop electrical excision procedure (LEEP) are effective but result in doubling of preterm delivery rate from 4.4% to 8.9%.^{4, 5} Therefore, the new treatment guidelines published in 2013 recommend 1-2 years of close observation in women, with cervical intraepithelial neoplasia 2, who are less than 25 years in age or who plan to have children at any age. For cervical intraepithelial neoplasia 3, treatment is recommended but observation is an accepted option.⁵ Non-surgical alternatives which would leave the cervix anatomically intact are needed but not currently available. When approved, an HPV therapeutic vaccine is likely to become the first-line therapy for treating CIN2/3 in young women. Furthermore, an HPV therapeutic vaccine, which requires only injections, could benefit women in developing regions where surgical expertise to perform excisional procedures may not be available.

HPV transformation of squamous epithelium to a malignant phenotype is mediated by two early gene products, E6 and E7,⁶ and their expression is necessary for HPV type 16 transformation of human cells.^{7, 8} T-cell responses to HPV type 16 E6 protein have been associated with favorable clinical outcomes such as viral clearance⁹ and regression of cervical lesions.^{10, 11} The E6 protein is an especially attractive target for immunotherapy since it is a viral protein, and attacking self-protein (i.e., autoimmunity) is not of concern.

Traditionally, recall antigens, which typically include a panel of Candida, mumps, and Trichophyton, were used as controls to indicate intact cellular immunity when patients were being tested for Tuberculosis by placement of PPD intradermally. T-cell mediated inflammation would become evident in 24-48 hours.¹² A number of studies have demonstrated that recall antigen injections can also be used to treat common warts (a condition also caused by HPV), and several studies have shown that treating warts with recall antigens is effective not only for injected warts but also distant untreated warts.¹³⁻¹⁶ This suggests that T-cells may have a role in wart regression. In a recently completed Phase I investigational new drug study (NCT00569231) in which the largest wart was treated with Candida, complete resolution of the treated warts was reported in 82% (nine of 11) of patients.¹⁶ Furthermore, T-cell responses to the HPV 57 L1 peptide were detected in 67% (six of nine) of the complete responders.¹⁶ These immune-enhancing and possible anti-HPV effects of Candida prompted the use of Candida as a vaccine adjuvant. Safety, efficacy, and immune responses of PepCan have been evaluated in a Phase I clinical trial (NCT01653249).

### Results

### Safety

Patient characteristics and adverse events (AEs) are summarized in Tables 3 and 4 respectively. None of the vaccine recipients experienced any dose-limiting toxicity, and the most frequent AEs were immediate (seen with all injections) and delayed injection site reactions. More grade 2 immediate and delayed injection site reactions were recorded at the higher doses [odds ratio of 33.0 (2.9, 374.3), p<0.0001 for the immediate reaction and odds ratio of 4.5 (0.9, 23.8), p=0.07 for the delayed reaction]. No patients discontinued due to AEs.

### Efficacy

CIN2/3 lesions are usually asymptomatic so vaccine response was assessed by histological regression. CIN2/3 was no longer present at exit in 9 of 23 (39%) patients who completed the study (Table 3), the remaining CIN2/3 lesions measured ≦0.2 mm² in 3 (13%) patients. The histological response rates by dose were 83%, 50%, 33%, and 40% with the best response at the lowest dose. None progressed to cervical squamous cell carcinoma. The regression rates were similar for CIN2 (50%) and CIN3 (62%), and in CIN2/3 associated and not associated with HPV 16 (44% vs. 57%). The mean number of cervical quadrants with visible lesions decreased significantly from 2.1±1.1 (range 0 to 4) quadrants prior to vaccination 0.8±1.0 (0 to 3) quadrants after vaccination (p<0.0001). However, five of the 12 subjects with no visible lesions after vaccination were histological non-responders with persistent CIN2/3. At least one HPV type present at entry became undetectable in 13 of 23 (57%) patients. By dose, the rates were 83%, 50%, 50%, and 40% with the highest undetectability at the lowest dose.

### Immune Responses

New CD3 T-cell responses to at least one region of the E6 protein were detected in 15 of 23 patients (65%, Table 3) with the increased responses after vaccination being statistically significant in 10 patients (43%). The CD3 T-cell response rates to E6 by dose were 83%, 67%, 83%, and 20% with the best responses at the 50 and 250 ug doses. The percentages of statistically significant increase in E6 responses were 50%, 50%, 50%, and 20% by dose. Patients 4 and 11 demonstrated statistically significant increases in one of the regions of E7 likely representing epitope spreading.

The percentages of regulatory T-cells (Tregs) were not changed after vaccinations while those of T-helper type 1 (Th1) cells were significantly increased (p=0.02). The percentages of T-helper type 2 (Th2) cells increased significantly initially after 2 vaccinations (p=0.03), but decreased below the baseline after 4 vaccinations (FIG. 6A). The differences between the responders and non-responders approached significance for Tregs at baseline (p=0.07) and at post-2 vaccinations (p=0.08, FIG. 6B). The number of Tregs infiltrating lesional cervical epithelium and the underlying stroma was lower in histological responders compared to non-responders, and approached statistical significance for the epithelium (p=0.08, FIG. 7).

### Medicinal Product

Precipitates became visible immediately at the 250 ug peptide dose-equivalent, and at other peptide dose-equivalents at 20 minutes. For peptides combined with Candida (CANDIN, Nielsen Biosciences, Inc., #59584-138-01) the precipitates formed at 20 minutes for the 500 ug peptide dose-equivalent, at 40 minutes for the 100 and 250 ug peptide dose-equivalents, and at 80 minutes for the 500 ug peptide dose-equivalent.

### HLA

Compared to the general population in the United States, HLA frequencies for A30, A33, A66, B14, B15, B40, C03, C18, DQ03, DQ05, and DR03 were significantly increased in patients who received vaccination (n=24). In order to eliminate the effect of disparate racial distributions between these two populations, expected HLA frequencies were calculated based on the racial distribution of the patients. Significant increases were observed in the patients for A32, B14, B15, B35, B40, C03, DQ03, and DR03. When the HLA frequencies were compared between histological responders and non-responders, B44 was significantly higher in responders (4 of 24 genes) compared to non-responders (0 of 22 genes, p=0.04).

TABLE 3
Subject characteristics, HPV types, T-cell response, and histological diagnoses at exit.

| Dose | No | Age | Race | HPV types at entry* | CD 3 T-cell responses in E6 detected after vaccination\hat{ } | Exit histology |
|---|---|---|---|---|---|---|
| 50 μg | 1 | 36 | Caucasian | 16, 52, 84 | None | CIN2,3 |
| | 2 | 49 | Caucasian | 45, 84 | 46-70 | CIN3^{#} |
| | 3 | 28 | Caucasian | 66, 84 | 16-40; 46-70 | No CIN |
| | 4 | 42 | African American | 45 | 1-25; 31-55; 46-70; 61-85; 76-100; 91-115; 106-130; 121-145 | CIN1 |
| | 5 | 31 | African American | 52, 53 61 | 16-40, 76-100; 91-115 | No CIN |
| | 6 | 41 | Caucasian | 16, 31, 58 | 1-25; 91-115; 136-158 | No CIN |
| 100 μg | 7 | 28 | African American | 26, 33, 51, 55, 58, 81 | 31-55; 106-130; 121-145; 136-158 | No CIN |
| | 8 | 22 | African American | 45, 56 | None | No CIN |
| | 9 | 34 | African American | 16 | 121-145; 136-158 | CIN2,3 |
| | 10 | 31 | African American | 35, 72, 83 | 16-40; 121-145; 136-158 | CIN2,3 |
| | 11 | 28 | African American | 16 | 1-25; 16-40; 31-55; 46-70; 61-85; 76-100; 91-115; 121-145; | CIN2 |
| | | | | | 136-158 | |
| | 12 | 32 | Mixed | 16 | None | No CIN |
| 250 μg | 13 | 29 | African American | 39, 73, IS39 | 106-130 | CIN2,3 |
| | 14 | 31 | African American | 58 | None | CIN2^{#} |
| | 15 | 32 | African American | 35 | 1-25 | CIN3 |
| | 16 | 25 | Caucasian | 16 | 16-40; 31-55; 46-70; 76-100; 91-115; 136-158 | CIN3 |
| | 17 | 22 | African American | 35, 59, 66, 81, | 1-25; 16-40; 46-70; 61-85; 76-100; 106-130; 121-145; 136-158 | CIN1 |
| | | | | CP6108 | | |
| | 18 | 23 | Caucasian | 45, 52, 62, 82 | 1-25; 31-55; 46-70; 61-85; 76-100; 91-115 | CIN3 |
| 500 μg | 19 | 29 | Caucasian | 16, 53 | 61-85; 91-115; 121-145 | CIN2,3 |
| | 20 | 26 | Caucasian | 16, 35, 58, 66 | None | CIN3^{#} |
| | 21 | 23 | African American | 58 | None | CIN3 |
| | 22 | 27 | Caucasian | 6, 52, 66, CP6108 | None | CIN2 |
| | 23 | 26 | African American | 31, 35 | NA | NA |
| | 24 | 32 | Caucasian | 16, 62 | None | No CIN |

*HPV types which became undetectable after vaccinations are shown in italics, and persistent HPV types are shown in bold.
\hat{ }CD3 T-cell response (positivity index ≧2.0 as long as at least 80 per 10⁶ IFN-g secreting CD3 cells detected) in new E6 region(s) after vaccinations.
^{#}considered to be a partial responder as the area of CIN3 measured ≦0.2 mm²
NA = not applicable

TABLE 4
A. Summary of adverse events

| | CTCAE Grade, Number of Events (Number of Patients) | | | | | | | |
|---|---|---|---|---|---|---|---|---|
| | Grade 1 | | | | Grade 2 | | | |
| Dose (ug/peptide) | 50 | 100 | 250 | 500 | 50 | 100 | 250 | 500 |
| Adverse event | | | | | | | | |
| Injection site reaction, | 23(6) | 24(6) | 18(6) | 11(6) | 1(1) | | 6(3) | 11(6) |
| immediate^{a} | | | | | | | | |
| Injection site reaction, other, | 5(4) | 4(3) | 3(3) | 4(3) | 1(1) | 1(1) | 3(1) | 5(4) |
| delayed^{b} | | | | | | | | |
| Myalgia | 8(3) | 4(1) | 4(1) | 4(3) | | | | 1(1) |
| Fatigue | 5(3) | 1(1) | 2(1) | 2(2) | | 1(1) | | |
| Diarrhea | 1(1) | | | | | | | |
| Nausea | 2(2) | 5(3) | | 5(4) | | | | |
| Vomiting | | | | 1(1) | | | | |
| Headache | 3(2) | 3(3) | 5(2) | 6(2) | | | | 2(1) |
| Pain - body | 2(2) | | | | 1(1) | | | 2(1) |
| Alopecia | | | | | 1(1) | | | |
| Feverish^{c} | 1(1) | 2(1) | 1(1) | 1(1) | | | | |
| Hot flashes | | | | 1(1) | | | | |
| Muscle spasm | 1(1) | | | | | | | |
| Flu-like symptoms | 4(1) | 3(1) | | | | | 1(1) | |
| Photophobia | | | | 1(1) | | | | |
| Agitation | | | 1(1) | 1(1) | | | | |
| Vertigo | | | 1(1) | | | | | |
| Dizziness | | | | 1(1) | | | | |
| Neutropenia | 1(1) | | | | | | | |
| Hypokalmia | 4(4) | 1(1) | 2(2) | 1(1) | | | | 1(1) |
| Thrombocytopenia | | | | 1(1) | 1(1) | | | |
| GGT increased | | 1(1) | | | | | | |

B. Detailed descriptions of injection site reactions

| | CTCAE Grade, Number of Events, (Number of patients) | | | | | | | |
|---|---|---|---|---|---|---|---|---|
| | Grade 1 | | | | Grade 2 | | | |
| Dose (ug/peptide) | 50 | 100 | 250 | 500 | 50 | 100 | 250 | 500 |
| Adverse Event | | | | | | | | |
| Injection site reaction, | 23(6) | 24(6) | 18(6 | 11(6) | 1(1) | | 6(3) | 11(6) |
| immediate | | | | | | | | |
| Pain | | | | | 1(1) | | 6(3) | 11(6) |
| Redness | 24(6) | 23(6) | 24(6) | 22(6) | | | | |
| Swelling | 2(1) | 7(2) | 1(1) | 8(4) | | | | |
| Welt | 7(4) | 16(5) | 22(6) | 21(6) | | | | |
| Tenderness | | | | 1(1) | | | | |
| Itching | 13(5) | 13(5) | 11(5) | 9(4) | | | | |
| Burning | | 1(1) | 1(1) | 1(1) | | | | |
| Warmness | | | 1(1) | 1(1) | | | | |
| Injection site reaction, | 5(4) | 4(3) | 3(3) | 4(3) | 1(1) | 1(1) | 3(1) | 5(4) |
| delayed | | | | | | | | |
| Pain | | | | | 1(1) | 1(1) | 3(1) | 5(4) |
| Redness | 5(4) | 2(2) | 5(3) | 3(3) | | | | |
| Swelling | 5(4) | 2(2) | 2(2) | 5(5) | | | | |
| Welt | | | | | | | | |
| Tenderness | | | | | | | | |
| Itching | 1(1) | 2(2) | 3(3) | 4(4) | | | | |
| Burning | | 2(1) | | | | | | |
| Warmness | | | | 1(1) | | | | |

^{a}appearing <24 hours from time of vaccination;
^{b}appearing >24 hours from time of vaccination;
^{c}feeling warm without evidence of temperature >38.0 C.

### Discussion

The safety of this HPV therapeutic vaccine has been demonstrated as no dose-limiting toxicities were reported. The most common AEs were immediate injection site reactions which were reported with all vaccinations. In contrast, only very rare observations of immediate reactions were recorded when Candida alone was injected for treating common warts.¹⁶ Therefore, the peptides are likely to be the culprit. These AEs may be related to the peptides' property of forming microparticles when placed in a neutral pH, although they are stably soluble in its formulation which has pH of 4. These microparticles would likely enhance the immunogenicity of the vaccine as they may stimulate Langerhans cells to phagocytose them.¹⁷ The unexpected AEs were delayed injection site reactions, which were defined as occurring equal to or more than 24 hours after injections. However, they appeared from 1 to 6 days afterwards and therefore not all of them could be dismissed as delayed-type hypersensitivity reactions.¹² The timing of occurring several days afterwards raises a possibility of de novo immune responses occurring at the site.¹⁸

The best histological regression rate was recorded with the 50 ug group (83%) while the overall regression rate was 52%. Both rates were higher than the 22% regression rate reported for a historical placebo group in another clinical trial of HPV therapeutic vaccine with a similar study design.¹⁹ Kenter et al. reported the complete histological regression rate of 25% at 3 months and 47% at 12 months in patients with HPV 16-positive high-grade vulvar intraepithelial lesions who received another peptide-based HPV therapeutic vaccine.²⁰ Therefore, the vaccine response is expected to increase with the extended observation period of 12 months which is being planned for the Phase II clinical trial.

New HPV 16 E6-specific CD3 T-cell responses were observed in 65% of patients and more than half had statistically significant increases, attesting to the immunogenicity of PepCan. Others have reported significant correlations between HPV therapeutic vaccine-induced immune responses and clinical outcomes.^{20, 21} Kenter et al. reported significantly higher numbers of interferon-γ producing CD4 T-cells and stronger proliferative responses in patients with complete responses compared to those with no responses at 3 months.²⁰ In a clinical trial of imiquimod and HPV therapeutic vaccination treating vulvar intraepithelial lesions, Daayana et al. found significantly increased lymphocyte proliferation to the HPV vaccine antigens in responders.²¹ We found no significant association between CD3 T-cell responses and histological regression as five responders had no new responses against E6. This may be due to a limitation of peripheral detection as HPV-specific T-cells would eventually need to reach the cervix to carry out their anti-HPV activity.

Epitope spreading is a process in which antigenic epitopes distinct from and non-cross-reactive with an inducing epitope become additional targets of an ongoing immune response, and it has been associated with favorable clinical outcomes for cancer immunotherapy.²² Two vaccine recipients demonstrated significant increases in T-cell response to HPV type 16 E7 protein in addition to the E6 protein contained in the vaccine. One had persistent HPV type 16 infection, and the other one had persistent HPV type 45 infection. As there is little amino acid homology between the E7 proteins of HPV types 16 and 45, this patient may have had a latent HPV type 16 infection undetectable by the PCR method or may have had a reactivation of memory T-cell response from her past HPV type 16 infection. HPV 16 is the most common HPV type detected,²³⁻²⁷ and a lifetime risk of acquiring HPV 16 is estimated to be 50%.²⁸

As an investigational adjuvant, granulocyte monocyte colony-stimulating factor has been reported to inadvertently increase Tregs resulting in less effective vaccine response.²⁹ Therefore, we monitored levels of Tregs, which were minimally changed. Th1 cells were significantly increased, supporting the immunostimulatory effect of PepCan. Our earlier work showed that Candida has T-cell proliferative effects, and that the cytokine most frequently produced by Langerhans cells exposed to Candida was interleukin-12 (IL-12).^{17, 30} Therefore, Candida is likely responsible for the increased levels of Th1 cells after vaccination, and may be an effective vaccine adjuvant for other therapies designed to promote T-cell activity, not only for other pathogenic antigens but also for tumor antigens in new cancer immunotherapies. Th1 polarization of T helper cells by IL-12 has been demonstrated previously in vitro³¹ and in a murine model.³² However, this is the first example, to our knowledge, of Th1 promotion due to an agent that likely induces IL-12 secretion in vivo. IL-12 is also known to be a potent inducer of antitumor activity.³³ Given the demonstrated safety profile of PepCan, this may be an effective alternative to systemic administration of IL-12 with which toxicities have been problematic.³³ Although Treg levels were not increased after vaccination, they may have an effect on whether subjects would respond to the vaccine, as pre-vaccination Treg levels were lower in non-responders compared to responders, though not significantly. This difference persisted over time. Therefore, it is possible that some pretreatment to decrease Treg levels prior to vaccine initiation such as administration of cyclophosphamide^{34, 35} may improve vaccine response. Treg levels were also higher in non-responders compared to responders in the cervical lesions and the underlying stroma (though the differences were not statistically significant) possibly supporting the negative role of Treg in vaccine response.

HLA gene frequencies of B14, B15, B40, C03, DQ03, and DR03 molecules were significantly higher in our patients compared to the general population in the United States and the general population adjusted for the racial distribution of the patients. Increased risk of cervical neoplasia associated with DQ03 has been reported by others.³⁶⁻³⁸ When histological responders and non-responders were compared only B44 was significantly elevated in responders compared to non-responders. This implies that the B44 molecule may present effective epitopes of HPV 16 E6 protein. However, no such epitopes have been described to date to our knowledge.

Unexpectedly, histological regression, undetectability of at least one HPV type present at entry, and immune responses were all superior at the lowest dose compared to the highest dose, and we plan to use the lowest dose for the Phase II clinical trial. As the number of subjects in each dose group was small (n=6), this study was not powered to show significant differences. As no patient with percent Treg equal to greater than 0.8% prior to vaccination responded, it is possible that the higher prevaccination Treg levels at higher doses may have influenced the outcome. The median percentages of Tregs were 0.5, 0.4, 0.7 and 0.9 by dose respectively. Nevertheless, we have shown that PepCan is safe and well tolerated, and a Phase II clinical trial in which the observation period is extended to 12 months for maximal response is warranted.

### PATIENTS AND METHODS

### Patients

This clinical trial was a Phase I single-arm, single-site, dose escalation study. Patients (n=37) were enrolled between September 2012 and March 2014, and those with biopsy-proven CIN2/3 (n=24) were eligible for vaccination (Table 3).

Vaccination was started within 60 days of biopsy date, and 4 injections were given 3 weeks apart. Each patient received the same dose of the peptides, and 6 subjects each were recruited in each dose group.

At the screening visit, the cervix was visualized under a colposcope after applying acetic acid, biopsies were obtained, Thin-Prep (Hologic, #70097-0001) was collected for HPV-DNA testing (Linear Array HPV Genotyping Test, Roche Molecular Diagnostics, #04472209190 and #03378012190), and routine laboratory testing was performed (complete blood count, sodium, potassium, chloride, carbon dioxide, blood urea nitrogen, creatinine, aspartate transaminase, alanine transaminase, lactate dehydrogenase, γ-glutamyl transpeptidase, total bilirubin, and direct bilirubin). Patients who already were diagnosed with biopsy-proven CIN2/3 were also eligible as long as the first vaccine injection could be given within 60 days, and other inclusion criteria were met (ages 18 to 50 years old, blood pressure ≦200/120 mm Hg, heart rate 50 to 120 beats per minute, respiration ≦25 breaths per minute, temperature ≦100.4° F., white count ≧3×10⁹/L, hemoglobin ≧8 g/dL, and platelet count ≧50×10⁹/L). Being positive for HPV 16 was not required due to possible cross-protection^{10, 11, 39, 40} and de novo immune stimulation.^{14, 16} Exclusion criteria included a history of disease or treatment causing immunosuppression, pregnancy, breast feeding, allergy to Candida, a history of severe asthma, current use of beta-blocker, and a history of invasive squamous cell carcinoma of the cervix. Urine pregnancy test was performed prior to each injection, and blood was drawn for routine laboratory testing and immunological assessments immediately prior to the first and third injections. The vaccine was administered intradermally in any limb. Twelve weeks after the last injection, blood was drawn, ThinPrep sample was collected, and LEEP was performed. Safety and tolerability were assessed from the time informed consent was obtained until the day LEEP was performed using version 4.1 of the National Cancer Institute Common Terminology Criteria for Adverse Events. Dose-limiting toxicities were defined as vaccine-related allergic and autoimmune AEs greater than grade 1 and any other AEs greater than grade 2. Efficacy was based on histological grading of the LEEP samples. A patient with no dysplasia or CIN 1 was considered to be a complete responder, and a patient with CIN2/3 measuring ≦0.2 mm² was considered to be a partial responder. The study was approved by the Institutional Review Board, and a written informed consent was obtained from each participant.

### Vaccine Composition

The vaccine consisted of four current good manufacturing production-grade synthetic peptides covering the HPV 16 E6 protein with the following sequences:

### E6 1-45 (Ac-MHQKRTAMFQDPQERPRKLPQLCTELQTTIHDIILECVYCKQQLL-NH2 (SEQ ID NO:2)),

### E6 46-80 (Ac-RREVYDFAFRDLCIVYRDGNPYAVCDKCLKFYSKI-NH2 (SEQ ID NO:3)),

### E6 81-115 (Ac-SEYRHYCYSLYGTTLEQQYNKPLCDLLIRCINCQK-NH2 (SEQ ID NO:4)), and

E6 116-158 (Ac-PLCPEEKQRHLDKKQRFHNIRGRWTGRCMSCCRSSRTRRETQL-NH2 (SEQ ID NO:5)).¹⁷ The two regions (amino acids 46-70 and 91-115) previously shown to be most immunogenic in terms of CD8 T-cell responses were preserved.¹¹

Reconstituted peptides alone or reconstituted peptides with Candida, at the same proportions as in the four doses being tested (but one sixth in total volume), were combined with RPMI1640 media (Mediatech, Inc., #10-040-CV) with 10% fetal calf serum (Atlanta Biologicals, #S11150H) in a 24 well plate. A total volume for each condition was 1 ml. The mixtures were incubated at 37° C. with 5% carbon dioxide. Visual inspection to detect precipitate formation was performed every 20 minutes for the first 80 minutes, and every 40 minutes for the following 160 minutes. Photomicrographs were taken at 24 hours using AxioCam Mrc5 attached to AxioImager Z1 with Axio Vision software (Carl Zeiss AG) in the University of Arkansas for Medical Sciences Digital Microscopy Laboratory.

Prior to injecting patients, lyophilized peptides were reconstituted with sterile water and were mixed with 300 ul of Candida albicans skin test reagent (CANDIN) in a syringe. The amount of peptide per injection was 50, 100, 250, or 500 ug per peptide, and the total injection volume was 0.4, 0.5, 0.75, or 1.2 ml respectively.

### Immunological Assessments

Peripheral HPV 16-Specific Cell Responses (Also See Supplementary Appendix)

T-cell lines were established from three blood draws from each patient as described previously with minor modifications.^{10, 11, 41} In short, peripheral blood mononuclear cells (PBMCs) were isolated from heparinized whole blood using a Ficoll density-gradient centrifugation method, separated into CD14+ monocytes and CD14-depleted PBMCs, and cryopreserved. Autologous dendritic cells were established by growing monocytes in the presence of granulocyte monocyte-colony stimulating factor (50 ng/mL, Sanofi-Aventis, #420039) and recombinant interleukin-4 (100 U/mL, R&D Systems, #204-IL-050) for seven days, and were matured by 48-hour culture in wells containing irradiated mouse L-cells expressing CD40 ligands. CD3 T-cells were magnetically selected (Pan T Cell Isolation Kit II, Miltenyi Biotec, #130-096-535) from CD14-depleted PBMCs. HPV 16 E6- and E7-specific CD3 T-cell lines were established by in vitro stimulation of CD3 cells for seven days with autologous dendritic cells pulsed with E6-glutathione S-transferase and E6 expressing recombinant vaccinia virus or E7-glutathione S-transferase and E7 expressing recombinant vaccinia virus.^{10, 11, 41-43} In vitro stimulation was repeated for an additional seven days.

ELISPOT assays were performed in triplicate using overlapping peptides covering the E6 and E7 proteins of HPV 16, as described.⁴¹ MultiScreen-MAHA plates (Millipore, #MAHAS4510) were coated with mouse anti-human interferon-gamma monoclonal primary antibody (5 ug/mL, 1-D1K, Mabtech, #3420-3-1000). The coated plates were washed and blocked. After incubating at 37° C. for 1 hour, 2.5×10⁴ CD3+ cells per well were added, along with pools of peptides (10 uM each) in triplicate. Negative control wells contained medium only, and positive control wells contained phytohaemagglutinin at 10 ug/mL (Remel, #R30852801). Following a 24 hour incubation, the plates were washed and a secondary antibody was added (1 ug/mL of biotin-conjugated anti-IFN-y monoclonal antibody; 7-B6-1, Mabtech, #3420-6-250). After a 2 hour incubation and washing, avidin-bound biotinylated horseradish peroxidase (Vectastain ABC Kit, Vector Laboratories, #PK-6100) was added. After 1 hour of incubation, the plates were washed, and stable diaminobenzene (50 uL, Life Technologies, #750118) was added. After developing the reaction for 5 minutes, the plates were washed with deionized water. Spot-forming units were be counted by an automated ELISPOT analyzer (AID ELISPOT Classic Reader; Autoimmun Diagnostika GmbH). An HPV-specific T-lymphocyte response was considered to be positive if spot-forming units in peptide containing wells were at least two times higher than in the corresponding negative-control wells (i.e., positivity index of ≧2.0),⁴⁴ and if at least 80 spot-forming units per 10⁶ CD3 T-cells were present in peptide containing wells. If any region was found to be positive after 2 or 4 vaccinations, and the positivity index was higher than that at the baseline, the number of peptide-specific spot forming units for each well was calculated by subtracting the number of background spot forming units from the negative control wells containing media only. Paired t-test was used to assess the significance of differences after 2 or 4 vaccinations compared to the baseline.

Peripheral Immune Cells

Thawed PBMCs were stained with relevant isotype controls and combinations of monoclonal antibodies to analyze Th1, Th2, and Tregs: fluorescein isothiocyanate-labeled anti-human CD4 (clone RPA-T4, eBioscience, #45-0048-41), phycoerythrin-labeled anti-human/mouse T-bet (clone 4B10, eBioscience, #12-5825-82), PerCP-Cy5.5-labeled anti-human CD25 (clone BC96, eBioscience, #45-0259-42), allophycocyanin-labeled anti-human Foxp3 (clone PCH101, eBioscience, #17-4776-42), and phycoerythrin-Cy7 labeled anti-human/mouse GATA3 (clone L50-823, Becton Dickinson Biosciences, #560405). Cells were first stained with antibodies for surface markers CD3, CD4, and CD25. Staining for intracellular T-bet, GATA3, and Foxp3 was performed using the Foxp3 staining kit (eBioscience, #00-5523-00) according to the manufacturer's instructions. Flow cytometric analysis was performed with FACS Fortessa using FACS Diva software (Becton Dickinson Biosciences) in the University of Arkansas for Medical Sciences Microbiology and Immunology Flow Cytometry Core Laboratory. Ten thousand events were acquired in the lymphocyte gate. CD4 cells were expressed as a percentage of lymphocytes, Th1 cells were expressed as a percentage of CD4 cells positive for Tbet, Th2 cells were expressed as a percentage of CD4 cells positive for GATA3, and regulatory T-cells were expressed as a percentage of CD4 cells positive for CD25 and Foxp3.¹⁰

Cervical Regulatory T-Cells

Nuclear localization of FoxP3 was utilized to quantitate Tregs using a digital pathology system.^{45, 46} Slides of LEEP samples were pretreated with a target retrieval solution (Dako Corporation, #S2369), peroxidase block (Dako Corporation, #S2003), and serum-free protein block (Dako Corporation, #X0909) prior to performing immunohistochemistry with primary goat anti-human polyclonal antibody against FoxP3 (R&D Systems, #AF3240) at 1:400 dilution. Following treatment with biotinylated rabbit anti-goat secondary antibody at 1:400 dilution (Vector Laboratories, #BA-5000), the slides were developed using Vectastain Elite ABC (Vector Laboratories, #PK-6100) and diaminobenzidine (Dako Corporation, #K3468). Hemaoxylin (Richard-Allan Scientific, #2-7231) was used as a counterstain. Using a digital pathology system (ScanScope® CS and ImageScope™ software, Aperio), lesions in the epithelium (minimum ≧0.2 mm²) and areas in the underling stroma (minimum ≧0.2 mm²) were marked by a study pathologist. Representative normal regions were selected if no lesions remained. Cells with positive nuclear staining were counted using the software.

### HLA Typing

Low-resolution typing for HLA class I A, B, and C and class II DRB1, DQB1, and DPB1 was performed with MicroSSP Generic DNA Typing Trays (One Lambda, #SSP1L and #SSPDRQP1), using DNA extracted from PBMCs. Data were analyzed with HLA Fusion (One Lambda).

### Statistical Analysis

A generalized estimate equation analyses were performed to compare the frequencies of grade 2 immediate and delayed injection site reactions between the higher doses (250 and 500 ug) and the lower doses (50 and 100 ug), while accounting for the correlation among injections given to the same individual. A sign test was performed to compare the numbers of cervical quadrants with visible lesions prior to and after 4 vaccinations. A paired t-test was used to determine significance of increased CD3 T-cell responses as determined by rising positivity index for each region after 2 or 4 vaccinations, and to compare percentages of Th1, Th2, and Tregs after 2 or 4 vaccinations from the baseline. Wilcoxon rank-sum test was used to compare percentages of Th1, Th2, or Tregs between responders and non-responders prior to vaccination, after 2 vaccinations or after 4 vaccinations. Chi-square test was used to compare frequencies of each HLA molecule between the patients and the general population in the United States or between the patients and the corrected population frequencies based on racial distributions of the patients.⁴⁷ Fisher's exact text was used to compare HLA frequencies between responders and non-responders. No adjustments were made for multiple comparisons.

### References For Example 3

1. Cancer IAfRo. GLOBOCAN 2012 CANCER FACT SHEET. Cedex, France, 2012.
2. Chaturvedi A K. Beyond cervical cancer: burden of other HPV-related cancers among men and women. J Adolesc Health 2010; 46:S20-6.
3. Tota J E, Chevarie-Davis M, Richardson L A, Devries M, Franco E L. Epidemiology and burden of HPV infection and related diseases: implications for prevention strategies. Prey Med 2011; 53 Suppl 1:S12-21.
4. Bruinsma F J, Quinn M A. The risk of preterm birth following treatment for precancerous changes in the cervix: a systematic review and meta-analysis. BJOG 2011; 118:1031-41.
5. Massad L S, Einstein M H, Huh W K, Katki H A, Kinney W K, Schiffman M, Solomon D, Wentzensen N, Lawson H W. 2012 updated consensus guidelines for the management of abnormal cervical cancer screening tests and cancer precursors. Obstet Gynecol 2013; 121:829-46.
6. Kumar V, Fausto N, Abbas A. Cervix. Robbins & Cotran Pathologic Basis of Disease, 2004:1072-9.
7. Pirisi L, Yasumoto S, Feller M, Doniger J, DiPaolo J. Transformation of human fibroblasts and keratinocytes with human papillomavirus type 16 DNA. J Virol 1987; 61:1061-6.
8. Schlegel R, Phelps W C, Zhang Y L, Barbosa M. Quantitative keratinocyte assay detects two biological activities of human papillomavirus DNA and identifies viral types associated with cervical carcinoma. Embo J 1988; 7:3181-7.
9. Nakagawa M, Stites D P, Patel S, Farhat S, Scott M, Hills N K, Palefsky J M, Moscicki A B. Persistence of human papillomavirus type 16 infection is associated with lack of cytotoxic T lymphocyte response to the E6 antigens. J Infect Dis 2000; 182:595-8.
10. Kim K H, Greenfield W W, Cannon M J, Coleman H N, Spencer H J, Nakagawa M. CD4+ T-cell response against human papillomavirus type 16 E6 protein is associated with a favorable clinical trend. Cancer Immunol Immunother 2012; 61:63-70.
11. Nakagawa M, Gupta S K, Coleman H N, Sellers M A, Banken J A, Greenfield W W. A favorable clinical trend is associated with CD8 T-cell immune responses to the human papillomavirus type 16 e6 antigens in women being studied for abnormal pap smear results. J Low Genit Tract Dis 2010; 14:124-9.
12. Esch R E, Buckley C E, 3rd. A novel Candida albicans skin test antigen: efficacy and safety in man. J Biol Stand 1988; 16:33-43.
13. Clifton M M, Johnson S M, Roberson P K, Kincannon J, Horn T D. Immunotherapy for recalcitrant warts in children using intralesional mumps or Candida antigens. Pediatr Dermatol 2003; 20:268-71.
14. Horn T D, Johnson S M, Helm R M, Roberson P K. Intralesional immunotherapy of warts with mumps, Candida, and Trichophyton skin test antigens: a single-blinded, randomized, and controlled trial. Arch Dermatol 2005; 141:589-94.
15. Johnson S M, Roberson P K, Horn T D. Intralesional injection of mumps or Candida skin test antigens: a novel immunotherapy for warts. Arch Dermatol 2001; 137:451-5.
16. Kim K H, Horn T D, Pharis J, Kincannon J, Jones R, O'Bryan K, Myers J, Nakagawa M. Phase 1 clinical trial of intralesional injection of Candida antigen for the treatment of warts. Arch Dermatol 2010; 146:1431-3.
17. Wang X, Coleman H N, Nagarajan U, Spencer H J, Nakagawa M. Candida skin test reagent as a novel adjuvant for a human papillomavirus peptide-based therapeutic vaccine. Vaccine 2013; 31:5806-13.
18. Abbas A K, Lichtman A H, Pillai S. Cellular and Molecular Immunology. Philadelphia: Elsevier, 2011.
19. Nieminen P, Harper D M, Einstein M H, Garcia F, Donders G, Huh W, Wright T C, Stoler M, Ferenczy A, Rutman O, et al. Efficacy and safety of RO5217990 treatment in patients with high grade cervical intraepithelial neoplasia (CIN2/3). 28th International Papillomavirus Conference. Puerto Rico, 2012.
20. Kenter G G, Welters M J, Valentijn A R, Lowik M J, Berends-van der Meer D M, Vloon A P, Essahsah F, Fathers L M, Offringa R, Drijfhout J W, et al. Vaccination against HPV-16 oncoproteins for vulvar intraepithelial neoplasia. N Engl J Med 2009; 361:1838-47.
21. Daayana S, Elkord E, Winters U, Pawlita M, Roden R, Stern P L, Kitchener H C. Phase II trial of imiquimod and HPV therapeutic vaccination in patients with vulval intraepithelial neoplasia. Br J Cancer 2010; 102:1129-36.
22. Ribas A, Timmerman J M, Butterfield L H, Economou J S. Determinant spreading and tumor responses after peptide-based cancer immunotherapy. Trends Immunol 2003; 24:58-61.
23. Bruni L, Diaz M, Castellsague X, Ferrer E, Bosch F X, de Sanjose S. Cervical human papillomavirus prevalence in 5 continents: meta-analysis of 1 million women with normal cytological findings. J Infect Dis 2010; 202:1789-99.
24. Clifford G M, Gallus S, Herrero R, Munoz N, Snijders P J, Vaccarella S, Anh P T, Ferreccio C, Hieu N T, Matos E, et al. Worldwide distribution of human papillomavirus types in cytologically normal women in the International Agency for Research on Cancer HPV prevalence surveys: a pooled analysis. Lancet 2005; 366:991-8.
25. Clifford G M, Smith J S, Plummer M, Munoz N, Franceschi S. Human papillomavirus types in invasive cervical cancer worldwide: a meta-analysis. Br J Cancer 2003; 88:63-73.
26. Munoz N, Bosch F X, de Sanjose S, Herrero R, Castellsague X, Shah K V, Snijders P J, Meijer C J. Epidemiologic classification of human papillomavirus types associated with cervical cancer. N Engl J Med 2003; 348:518-27.
27. Smith J S, Lindsay L, Hoots B, Keys J, Franceschi S, Winer R, Clifford G M. Human papillomavirus type distribution in invasive cervical cancer and high-grade cervical lesions: a meta-analysis update. Int J Cancer 2007; 121:621-32.
28. van den Hende M, Redeker A, Kwappenberg K M, Franken K L, Drijfhout J W, Oostendorp J, Valentijn A R, Fathers L M, Welters M J, Melief C J, et al. Evaluation of immunological cross-reactivity between clade A9 high-risk human papillomavirus types on the basis of E6-Specific CD4+ memory T cell responses. J Infect Dis 2010; 202:1200-11.
29. Slingluff C L, Jr., Petroni G R, Olson W C, Smolkin M E, Ross M I, Haas N B, Grosh W W, Boisvert M E, Kirkwood J M, Chianese-Bullock K A. Effect of granulocyte/macrophage colony-stimulating factor on circulating CD8+ and CD4+ T-cell responses to a multipeptide melanoma vaccine: outcome of a multicenter randomized trial. Clin Cancer Res 2009; 15:7036-44.
30. Nakagawa M, Coleman H N, Wang X, Daniels J, Sikes J, Nagarajan U M. IL-12 secretion by Langerhans cells stimulated with Candida skin test reagent is mediated by dectin-1 in some healthy individuals. Cytokine 2014; 65:202-9.
31. Manetti R, Parronchi P, Giudizi M G, Piccinni M P, Maggi E, Trinchieri G, Romagnani S. Natural killer cell stimulatory factor (interleukin 12 [IL-12]) induces T helper type 1 (Th1)-specific immune responses and inhibits the development of IL-4-producing Th cells. J Exp Med 1993; 177:1199-204.
32. Hsieh C S, Macatonia S E, Tripp C S, Wolf S F, O'Garra A, Murphy K M. Development of TH1 CD4+ T cells through IL-12 produced by Listeria-induced macrophages. Science 1993; 260:547-9.
33. Tugues S, Burkhard S H, Ohs I, Vrohlings M, Nussbaum K, Vom Berg J, Kulig P, Becher B. New insights into IL-12-mediated tumor suppression. Cell Death Differ 2014.
34. Berd D, Maguire H C, Jr., Mastrangelo M J. Potentiation of human cell-mediated and humoral immunity by low-dose cyclophosphamide. Cancer Res 1984; 44:5439-43.
35. Emadi A, Jones R J, Brodsky R A. Cyclophosphamide and cancer: golden anniversary. Nat Rev Clin Oncol 2009; 6:638-47.
36. Hildesheim A, Schiffman M, Scott D R, Marti D, Kissner T, Sherman M E, Glass A G, Manos M M, Lorincz A T, Kurman R J, et al. Human leukocyte antigen class I/II alleles and development of human papillomavirus-related cervical neoplasia: results from a case-control study conducted in the United States. Cancer Epidemiol Biomarkers Prey 1998; 7:1035-41.
37. Madeleine M M, Johnson L G, Smith A G, Hansen J A, Nisperos B B, Li S, Zhao L P, Daling J R, Schwartz S M, Galloway D A. Comprehensive analysis of HLA-A, HLA-B, HLA-C, HLA-DRB1, and HLA-DQB1 loci and squamous cell cervical cancer risk. Cancer Res 2008; 68:3532-9.
38. Wang S S, Wheeler C M, Hildesheim A, Schiffman M, Herrero R, Bratti M C, Sherman M E, Alfaro M, Hutchinson M L, Morales J, et al. Human leukocyte antigen class I and II alleles and risk of cervical neoplasia: results from a population-based study in Costa Rica. J Infect Dis 2001; 184:1310-4.
39. Kim K H, Dishongh R, Santin A D, Cannon M J, Bellone S, Nakagawa M. Recognition of a cervical cancer derived tumor cell line by a human papillomavirus type 16 E6 52-61-specific CD8 T cell clone. Cancer Immun 2006; 6:9.
40. Wang X, Greenfield W W, Coleman H N, James L E, Nakagawa M. Use of Interferon-gamma Enzyme-linked Immunospot Assay to Characterize Novel T-cell Epitopes of Human Papillomavirus. J Vis Exp 2012.
41. Nakagawa M, Kim K H, Moscicki A B. Patterns of CD8 T-cell epitopes within the human papillomavirus type 16 (HPV 16) E6 protein among young women whose HPV 16 infection has become undetectable. Clin Diagn Lab Immunol 2005; 12:1003-5.
42. Nakagawa M, Kim K H, Gillam T M, Moscicki A B. HLA class I binding promiscuity of the CD8 T-cell epitopes of human papillomavirus type 16 E6 protein. J Virol 2007; 81:1412-23.
43. Wang X, Moscicki A B, Tsang L, Brockman A, Nakagawa M. Memory T cells specific for novel human papillomavirus type 16 (HPV16) E6 epitopes in women whose HPV16 infection has become undetectable. Clin Vaccine Immunol 2008; 15:937-45.
44. Kaul R, Dong T, Plummer F A, Kimani J, Rostron T, Kiama P, Njagi E, Irungu E, Farah B, Oyugi J, et al. CD8(+) lymphocytes respond to different HIV epitopes in seronegative and infected subjects. J Clin Invest 2001; 107:1303-10.
45. Kobayashi A, Weinberg V, Darragh T, Smith-McCune K. Evolving immunosuppressive microenvironment during human cervical carcinogenesis. Mucosal Immunol 2008; 1:412-20.
46. Magg T, Mannert J, Ellwart J W, Schmid I, Albert M H. Subcellular localization of FOXP3 in human regulatory and nonregulatory T cells. Eur J Immunol 2012; 42:1627-38.
47. Organ Procurement and Transplantation Network. U.S. Department of Health & Human Services.

### Example 4. A Phase II Clinical Trial of PepCan Randomized and Double-Blinded to Two Therapy Arms for Treating Cervical High-Grade Squamous Intraepithelial Lesions

### Need for HPV Therapeutic Vaccines

Although numerous preclinical and clinical trials have evaluated prophylactic HPV vaccines during the past few decades, these vaccines do not help those who already have established HPV infections[51]. Gardasil, a quadrivalent HPV L1 virus-like particle vaccine (HPV types 16, 18, 6, and 11), was the first to be FDA-approved in 2006; a bivalent version (HPV types 16 and 18), Cervarix, was approved by the FDA three years later. Clinical trials have demonstrated excellent vaccine efficacy in women negative for HPV 16 or HPV18[52, 53], but the duration of protection remains to be determined, and a study of the bivalent vaccine showed no evidence of enhanced viral clearance in women with pre-existing HPV infections (n=1,259; 35.5% clearance in vaccinated group, 31.5% in a group receiving a negative control vaccine, p=NS)[51]. Therefore, therapeutic vaccines are needed for cases in which HPV infection is already established and in which HPV-related diseases have already developed. This is the particularly true because the prophylactic vaccine coverage rate in the targeted group (girls aged 13-17 years) has been reported to be only 32% nationally[54]. Although the standard surgical treatments for HSILs such as LEEP are very effective[14], their unintended side effect of increased incidence of preterm delivery from 4.4% to 8.9%[14, 15] has become a concern. Henceforth, the latest guideline no longer recommends treatment for CIN2 in young women (narrowly defined as ≦24 years old and broadly defined as any women who still plans to become pregnant[14]). Treatment is still recommended for CIN3 but observation is now considered acceptable. A new treatment which does not alter the anatomical integrity of the cervix like the HPV therapeutic vaccine is very much needed. In short, HPV therapeutic vaccines are needed because (1) prophylactic vaccines are not effective against established HPV infection, (2) utilization of the prophylactic vaccines has been low, (3) therapeutic vaccines would leave the cervix intact and would likely not increase the risk of preterm deliveries, and (4) therapeutic vaccine maybe effective against other cancers caused by HPV such as anal, oropharyngeal, penile, vaginal, and vulvar cancers.

### 1.5.1 Overview

This is a Phase II study to evaluate the efficacy and safety of an HPV therapeutic vaccine called PepCan (HPV 16 E6 peptides combined with Candida skin testing reagent called CANDIN) in adult females over a 12 month time period. As the results from the Phase I trial demonstrated some efficacy against non-16 HPV types, CANDIN alone will also be tested. Therefore, there will two treatment arms: (1) PepCan and (2) CANDIN. Subjects found to be eligible for vaccination will be randomized in a double-blinded fashion at a 1:1 ratio. Each participant will be receiving injections four times with three weeks between injections. Clinical and virological responses will be assessed at 6 and 12 months. Safety will be assessed from the time of enrollment to 12 Month Visit. Immunological assessments will be made at 4 time points (prevaccination, after 2 injections, 6 month after 4 injections and 12 months after 4 vaccinations).

### 1.5.2 Rationale for Proposed Dose of HPV Peptides

In the Phase I clinical trial, four dose levels (50, 100, 250, and 500 ug per peptide) were tested. The dose level with the highest clinical response will be selected to be used in the Phase II clinical trial. Thus far, the 50 ug per peptide dose has a higher response rate (67% complete response and 17% partial response) compared to the 100 ug per peptide (50% complete response).

The initial four dose levels were chosen based on information available in the literature. Published studies of clinical trials using various peptide vaccines reported using doses that range from 5-3,000 ug per peptide[31-38]. Optimal doses (and smaller doses if two dose levels were the same) for achieving immunogenicity differed greatly among the vaccines: 30 ug of 96-mer malaria peptide[31], 500 ug of 9-mer peptide for treating prostate cancer[34], 50 ug each of 13 HPV 16 E6 and E7 peptides ranging from 25 to 35 amino acids long[35]. Therefore, the dose levels likely to elicit the optimal immunogenicity were chosen.

The dose-escalation portion of the Phase I clinical trial has demonstrated that the 50 ug/peptide/injection was optimal in terms of histological regression, viral clearance, and vaccine-induced immune responses (Table 3). Therefore, this dose will be used for the Phase II clinical trial.

### 1.5.3 Rationale for Proposed Dose of CANDIN

Three hundred (300) μl of CANDIN will be administered per injection, which was the amount used for intralesional injection of warts[47, 55], as well as the amount of CANDIN as a vaccine adjuvant in the Phase I clinical trial. The same amount will be used for the Phase II clinical trial as this amount has been shown to be safe and effective.

### 1.5.4 Rationale for Proposed Route of Injections

Intradermal route of administration will be used to make use of LCs as antigen-presenting cells. This route has also been shown to be safe, effective, and immunogenic in the Phase I clinical trial, and will be used for the Phase II clinical trial.

### 1.5.6 Rationale for Number of Injections

### 1.5.5 Rationale for Proposed Site of Injections

Extremities have been chosen as the site of administration because of the ease of access as well as availability of sufficient data demonstrating efficacy of HPV peptides delivered at these sites[35, 56]. As injecting in limbs has shown to be safe, effective, and immunogenic in the Phase I clinical trial, the same sites will be used for injection in the Phase II clinical trial.

1.5.7 Rationale for Number of Injections and Interval between Injections

In published studies of peptide vaccines, the total number of injections ranged from 2 to 17 [31-38]. We proposed to use four injections because Hueman et al. demonstrated that immunogenicity peaked after four injections (six injections in total were given in the study)[34], and four injections appeared to be sufficient in the Phase I clinical trial.

The interval between injections ranged from 2 weeks to 90 days in the published studies[31-38], but most used a 3-week interval. Kenter and colleagues reported that peptide vaccine immunogenicity measured by IFN-γ ELISPOT assay was less prevalent when blood samples were drawn 7 days after the last vaccination but was higher when they were drawn 3 weeks after the last vaccination[35]. Therefore, we chose the 3-week (±7 days) interval because it appears to be long enough to allow sufficient mounting of immune responses. As this interval has been shown to be safe, effective, and immunogenic, the same interval will be used in the Phase II clinical trial.

1.5.8 Rationale for Interval between the Last Injection and Final Histologic Assessment

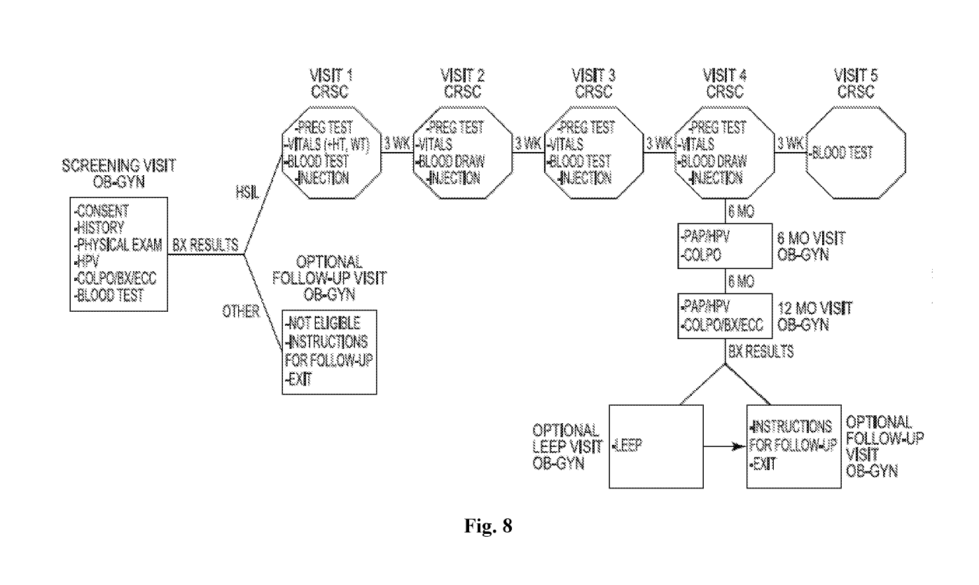

While histological response was assessed 3 months after the last vaccination by performing LEEP in the Phase I clinical trial, the full effect is known to take 1 year[17-19]. In the Phase II clinical trial, PepCan will be administered as an alternative to LEEP, and histological response will be assessed by obtaining colposcopy-guided quadrant biopsies 12 months after the last injection (FIG. 8). In a clinical trial which used a similar peptide-based HPV therapeutic vaccine to treat high-grade vulvar intraepithelial lesions, histological regression increased from 25% to 47% between 3 months and 12 months post-vaccinations[18].

### 1.5.9 Rationale for Primary Outcome Measure: Efficacy

The clinical response to evaluate the vaccine efficacy will be assessed by comparing the punch biopsy results between the Screening Visit (having had HSIL to qualify for vaccination) and the 12-Month Visit (±2 weeks). LEEP will not be performed to assess efficacy, but it will be offered at no cost to subjects who have persistent HSILs at the 12 Mo Visit.

The design of the proposed Phase II trial is single-site, and randomized to 2 treatment arms in a double-blinded fashion. We will use a historical placebo group from a clinical trial with similar design (i.e., enrollment of subjects with biopsy-proven CIN2/3, and clinical response assessed by biopsy in 15 month) for comparison[57]. The overall histological regression rate in the dose-escalation Phase I clinical trial was 52% three months after the last vaccination, and this is expected to substantially increase with an extended 12 month observation period.[18] Assuming a conservative rate of 60%, n=35 in the PepCan arm would give 91% power (two-tailed, α=0.05) for detecting a statistically significant difference from the historical placebo group which had a 29% (34 of 117) regression rate[57]. Although there is greater uncertainty regarding the CANDIN-only arm, there is ≧90% power to detect a significant differences between the PepCan and CANDIN arms under multiple plausible scenarios (for example, regression rates of 67% vs. 29%, or 85% vs. 50%). Forty subjects in each arm will be enrolled to ensure that at least 35 subjects in each would complete the study. While the use of historical placebo group is not as rigorous as having a concurrent placebo group, a concurrent placebo group with biopsy-proven CIN2/3 that would go untreated for 12 months would be difficult to ethically justify.

### 1.5.10 Rationale for Secondary Outcome Measure: Safety

The combination of HPV peptides and CANDIN was first tested in the Phase I clinical trial, and appears to be safe as no vaccine-related AEs >grade 2 have been reported (Table 4). Safety will be assessed in the same manner in the Phase II clinical trial using CTCAE 4.03.

### 1.5.11 Rationale for Tertiary Outcome Measures: Immunological Response and Viral Clearance

### 1.5.11.1 Rationale for Measuring HPV-specific T-Cell Response

HPV-specific CD3 T-cell responses will be assessed using immune assay such as the IFN-γ ELISPOT assay before vaccination, after 2 vaccinations, and 6 months after 4 vaccinations, and 12 months after 4 vaccinations. In order to evaluate the role of CD3 T-cells in vaccine efficacy, whether clinical response and viral clearance can be predicted based on the CD3 T-cell activities will be assessed.

### 1.5.11.2 Rationale for Measuring Circulating Immune Cells

The level of circulating immune cells, including CD4 T-cells, Th1 cells, Th2 cells, regulatory T-cells (Treg), and myeloid-derived suppressor cells (MDSC), will be assessed before vaccination, after 2 vaccinations, and after 4 vaccinations. Data from the Phase I clinical trial indicate that PepCan may increase Th1 responses (p=0.02) and decrease Th2 responses resulting in increased effector immune activity (FIG. 6B). Whether the levels of these circulating immune cells can be used to predict vaccine efficacy in terms of clinical response and viral clearance will be investigated.

### 1.5.11.3 Rationale for Measuring Viral Clearance

HPV-DNA testing will be performed at the Screening Visit, 6-Month Visit, and 12-Month Visit (FIG. 8). Thus far, all study participants had at least one HPV type at the Screening Visits. Clearance of at least one HPV type appears to correlate with clinical response. In the Phase II study, an HPV type would be considered to be cleared if it is present at the Screening Visit but not at the 6-Month and 12-Month Visits.

1.5.12 Rationale of Other Outcome Measures: Predict Vaccine Response Using Various Factors such as Age, HLA types, HPV types, Proteomics Profiling, Cytokine/Chemokine Profiling, and Laboratory Tests; Determine Cross-Protection and Examine Epitope Spreading and Cross-Reactivity as Possible Mechanisms

Not all vaccine recipients are expected to have clinical response. Some may have persistent HSIL, and some may progress to invasive squamous cell carcinoma. It would be valuable to identify factors that are associated with a favorable response so an educated decision can be made as to who should receive the vaccine, and how long one should wait before opting for surgical treatments. Therefore, a systems biology approach may be employed to determine factors that are associated with clinical response and viral clearance.

The Phase I clinical trial has indicated that PepCan is effective in HSILs with HPV 16 and non-16 HPV types. In the Phase II clinical trial, against which non-16 HPV types it is effective may be determined. Furthermore, epitope spreading and cross-reactivity may be investigated as possible mechanisms behind cross-protection.

### 1.5.13 Rationale for Adding a CANDIN Arm

The results of the dose-escalation portion of the Phase I clinical trial showed similar rates of clinical responses in subjects with HSILs associated (4 of 9 or 44%) and not associated (8 of 14 or 57%) with HPV 16 suggesting that de novo immune stimulation presumably from CANDIN plays a major role. Therefore, CANDIN only treatment arm will be added to compare efficacy between PepCan and CANDIN.

### 1.5.14 Rationale for Randomization and Double-Blinding

In order to minimize bias, subjects who are eligible for vaccination will be randomly assigned to one of the two treatment arms (PepCan or CANDIN) in a double-blinded fashion so the subjects and study staff (except for pharmacy staff) will not know which treatment is being administered. PepCan and CANDIN are both clear solutions prepared in the same 1 ml syringe.

### 2 Objectives

### Primary Objective: Efficacy 2.1

To assess the efficacy of PepCan and CANDIN in a Phase II clinical trial by determining clinical response which will be assessed by obtaining colposcopy-guided quadrant biopsis at the 12-Month Visit. If, upon the 12-Month visit biopsy, a subject does not have any evidence of CIN 2/3, she would be considered a “responder”. Some would have regressed to CIN 1, and others may have no dysplasia. If there is still CIN 2 and/or 3 present at the 12-Month Visit, the subject will be considered a “non-responder”.

### Secondary Objective: Safety 2.2

Safety will be assessed by documenting AEs from the time of enrollment until the 12-Month Visit according to CTCAE v4.03.

### Tertiary Objectives: Immunological Response and Viral Clearance 2.3

Immunological assessment in terms of HPV-specific CD3 T-cell responses will be assessed using an IFN-γ ELISPOT assay while circulating levels of CD4, Th1, Th2, Treg, and MDSC cells will be assessed by FACS analysis before vaccination, after 2 vaccinations and 6 months after 4 vaccinations, and 12 months after 4 vaccinations. Virological assessments will be made at Screening Visit, 6-Month Visit, and 12-Month Visit.

### Other Objectives 2.4

To evaluate predictive factors for response to the PepCan or CANDIN (in order to determine what specific group of women should receive the vaccine and timing of surgical treatments), various parameters such as age, HLA types, HPV types, proteomics profiling, cytokine/chemokine profiling, laboratory results, prophylactic HPV vaccination, tobacco use, oral contraceptive use, Pap smear results, CIN grade (CIN 2 vs. CIN 3), initial vital signs, body mass index, CD3 T-cell response to HPV 16 E6, and circulating immune cells may be analyzed.

Cross-protection in terms of clinical response may be determine by tallying each HPV event detected prior to vaccination in subjects who demonstrate HSIL regression for each of the 36 HPV types (other than 16) tested.

Cross-protection by PepCan and immune stimulation by CANDIN in terms of viral clearance may be determined by tallying each HPV event that is present at Screening Visit but becomes undetectable at both 6-Month and 12-Month Visits for each of the 36 HPV types tested.

Epitope spreading and cross-reactivity may be examined in selected subjects in the PepCan arm.

### 3 Investigational Product

### Test Article 3.1

### 3.3.1 HPV Peptides

The PepCan peptide mixture will contain four HPV 16 E6 peptides: E6 1-45 (Ac-MHQKRTAMFQDPQER PRKLPQLCTELQTTIHDIILECVYCKQQLL-NH2 (SEQ ID NO:2)), E6 46-80 (Ac-RREVYDFAFRDLCIV YRDGN PYA VCDKCLKFYSKI-NH2 (SEQ ID NO:3)), E6 81-115 (Ac-SEYRHYCYSLYGTTLEQQYNK PLCDLLIRCINCQK-NH2 (SEQ ID NO:4)), and E6 116-158 (Ac-PLCPEEKQRHLDKKQRFHNIRGRWT GRCMSCCRSSRTRRETQL-NH2 (SEQ ID NO:5)) (U.S. Pat. No. 8,652,482). Commercially produced cGMP-grade peptides (CPC Scientific, San Jose, Calif.) will be examined.

The four peptides will be provided in a single vial in lyophilized form, and will be stored at −70° C. (acceptable range −65° C. to −75° C.) except during shipping and immediately prior to use.

### 3.1.2 CANDIN

Candida Albicans Skin Test Antigen for Cellular Hypersensitivity will be supplied in the commercially marketed drug CANDIN. The vials will be stored at 2° C. to 8° C. as directed by the package insert until use. This product is approved for multi-dosing. The dose of CANDIN per injections for this study is 0.3 ml.

### 3.1.3 Combining HPV Peptides and CANDIN

Sterile water will be added to a vial containing the four cGMP peptides on the day of. Appropriate volume of reconstituted peptides will be drawn in a syringe depending on the dose level, and 0.3 ml of CANDIN will be drawn into the same syringe. The combined peptide-CANDIN mixture should be kept on ice or in refrigerator until immediately before injection.

### Treatment Regimen 3.2

Subjects will receive four injections of PepCan (50 μg/peptide/injection) via intradermal injection in the extremities with three weeks between each injection.

### 4 Study Design

### Overview 4.1

This is a single site Phase II clinical trial of PepCan for treating women with biopsy-proven HSILs randomized and double-blinded to two treatment arms. Half of the subjects will receive PepCan, and the other half will receive CANDIN alone. The study design closely resembles the latest guidelines for treating young women with HSIL[14]. Study participants will be patients attending the UAMS Obstetrics and Gynecology Clinics with untreated biopsy-proven HSILs and patients referred from other clinics. Four injections (one every 3 weeks) of PepCan or CANDIN will be intradermally administered in the extremities. Clinical response will be assessed by comparison of colposcopy-guided biopsy results obtained prior to vaccination and at 12 Month Visit. Safety will be monitored from the time of enrollment through the 12 Month Visit. Blood will be drawn for laboratory testing and immunological analyses (“blood test”) prior to injection, after the second vaccination, 6 months after the fourth vaccination, and 12 months after the fourth vaccination. Blood will be drawn to aid T-cell analyses (“blood draw”) after the first and third vaccinations, and possibly at the Optional Follow-Up and/or Optional LEEP visits. HPV-DNA testing will be performed at Screening and 6 and 12 Month Visits (FIG. 8). If a subject has persistent HSIL at the 12 Month Visit or if a subject is exited due to excessive toxicity, she will be given an option to return for a LEEP visit. Alternatively, she may choose to exit the study and be followed by her physician for up to 2 years of observation as recommended before surgical treatment[14].

### Monitoring Toxicity 4.2

Serious toxicity will be defined (using CTCAE v 4.03) as drug-related:
Grade II or higher allergic reactions. Grade II is defined as “intervention or infusion interruption indicated; responds promptly to symptomatic treatment (e.g., antihistamines, NSAIDS, narcotics); prophylactic medications indicated for ≦24 hours”. Grade III is defined as “prolonged (e.g., not rapidly responsive to symptomatic medication and/or brief interruption of infusion); recurrence of symptoms following initial improvement; hospitalization indicated for clinical sequelae (e.g., renal impairment, pulmonary infiltrates)”.
Grade II or higher autoimmune reactions. Grade II is defined as “evidence of autoimmune reaction involving a non-essential organ or function (e.g., hypothyroidism)”. Grade III is defined as “autoimmune reactions involving major organ (e.g., colitis, anemia, myocarditis, kidney)”.
Any Grade III or higher event.

Any subject who experiences serious toxicity will be discontinued from the study.

### Stopping Rules 4.3

A subject should be exited from the study at any point if pelvic examination and histological analysis show evidence of an invasive squamous cell carcinoma or if there is a clinical suspicion of having developed it based on signs and symptoms such as unexplained, prolonged vaginal bleeding.
The study enrollment and vaccine administration will be suspended if any subject experiences vaccine-related Grade IV or higher AE. These activities can re-start only after the Medical Monitor and applicable regulatory authorities grant permission.
The sponsor may decide to stop the study at any point, for any reason.

### 5 Subject Enrollment and Study Duration

### 5.1 Subject Population, Recruitment, and Informed Consent Process

Women, aged 18 to 50 years, seen at the UAMS Obstetrics and Gynecology Clinics and ANGELS Telecolposcopy program with recent Pap smear results positive for HSIL or “Cannot rule out HSIL” will be recruited through Physician referral, brochures, flyers, UAMS website, and word of mouth by study team; interested potential subjects will contact the study coordinator to discuss study; coordinator will conduct initial inclusion/exclusion criteria assessment, schedule subject for screening visit, and send a copy of the informed consent document for the subject to review
Other women with recent abnormal Pap smear results positive for HSIL or “Cannot rule out HSIL” will be recruited through clinic referral, brochures, flyers (distributed on and off campus), UAMS website, and advertisements in newspaper, radio, and/or social networking site; interested potential subjects will contact the study coordinator to discuss study; coordinator will conduct inclusion/exclusion criteria assessment, schedule subject for screening visit, and send a copy of the informed consent document for the subject to review; coordinator will request that subject obtain copy of Pap smear result from their physician's office and bring with them to the screening visit
Women with recent diagnosis (the duration between the day of diagnosis and the day of 1st injection needs to be ≦60 days) of HSIL on colposcopy guided punch biopsy will be recruited through clinic referral, brochures, flyers (distributed on and off campus), UAMS website, and advertisements in newspaper, radio, and/or social networking site; interested potential subjects will contact the study coordinator to discuss study; coordinator will conduct inclusion/exclusion criteria assessment, schedule subject for screening visit, and send a copy of the informed consent document for the subject to review; coordinator will request that subject obtain copies of medical records of abnormal biopsy from their physician's office and bring it with them to the screening visit

### 5.1.1 Inclusion Criteria

Aged 18-50 years
Had recent (≦60 days) Pap smear result consistent with HSIL or “cannot rule out HSIL” or HSIL on colposcopy guided biopsy
Untreated for HSIL or “Cannot rule out HSIL”
Able to provide informed consent
Willing and able to comply with the requirements of the protocol with a good command of the English language

### 5.1.2 Exclusion Criteria

History of disease or treatment causing immunosuppression (e.g., cancer, HIV, organ transplant, autoimmune disease)
Being pregnant or attempting to be pregnant within the period of study participation
Breast feeding or planning to breast feed within the period of study participation
Allergy to Candida antigen
History of severe asthma requiring emergency room visit or hospitalization
Current use of beta-blocker medication (may not respond to epinephrine in case of anaphylaxis)
History of invasive squamous cell carcinoma of the cervix
History of having received PepCan
If in the opinion of the Principal Investigator or other Investigators, it is not in the best interest of the patient to enter this study

### 5.1.3 Informed Consent Process

Potential subjects will be provided the informed consent form before the screening visit and allowed as much time needed to make decisions regarding study participation
The study coordinator/study team member authorized by PI to administer informed consent discussion will discuss the study in detail (including the age-specific standard of care guidelines as periodically released by the American Society of Colposcopy and Cervical Pathology) with the potential subject at any time before the screening visit or at a UAMS Gynecology clinic when she arrives for the screening visit (prior to any study-related procedures), and answer any questions the subject may have about the study; discussions will be conducted in English
As consent is an ongoing process, subjects will be asked if they still wish to participate in the study prior to study procedures conducted at each study visit

### Pace of Enrollment 5.2

During the Phase I study, approximately two thirds of subjects enrolled qualified for vaccination. Taking into account the screen-failure rate and attrition rate (currently about 5% per year), we plan to enroll 125 subjects for screening, and to initiate vaccination in 80 subjects.

### Study Duration 5.3

The study duration will be up to 66 months. Each subject is expected to be in the study for approximately 16 months or longer if LEEP excision is performed.

### 6 Study Visits

### Scheduling Study Visits 6.1

The Study Coordinator will schedule study visits (Screening, Vaccination, 6-Month, 12-Month, and Optional LEEP Visits) at the UAMS Obstetrics and Gynecology Clinics and the Clinical Research Services Core (CRSC). The Screening, 6-Month, 12-Month, and Optional LEEP Visits are expected to take approximately 90 minutes. However, they may be longer on busy clinic days. Vaccination Visits are expected to take approximately 60 minutes.

### Study Visit Windows 6.2

### 6.2.1 Between Visits of an Individual Subject

The first vaccination visit (Visit 1) should be scheduled as soon as possible after all results from the screening visit are available, and subjects are deemed qualified to continue to the vaccination phase of the study, but no later than 60 days after the day punch biopsy was obtained (the screening day for most of the subjects).
The subsequent vaccination/lab visits (Visits 2-5) should be scheduled 3 weeks±7 days apart.
The 6-Month visit should be scheduled 6 months+2 weeks following Visit 4
The 12-Month visit should be scheduled 6 months+2 weeks following 6-Month visit
Optional LEEP visit (if subject chooses) should be scheduled as soon as possible after 12-Month visit or after a subject is exited due to serious toxicity

### Screening Visit 6.4

### 6.4.1 Procedures for Screening Visit

Review inclusion/exclusion criteria
Obtain informed consent (if not previously obtained)
Have the subject fill out “Subject Contact Information” (Appendix 2) during the visit
Have the subject fill out “Screening Visit Questionnaire” (Appendix 3) during the visit
Obtain demographic information
Obtain subject's history
   Medical history: Be sure to ask for history of previous abnormal Pap smears and how they were treated
   Drug allergies
   Concomitant medications
Perform a physical examination
   Obtain vital signs
   Blood pressure (<200/120 mm Hg acceptable)
   Heart rate (50-120 beats per min acceptable)
   Respiratory rate (<25 breaths per min acceptable)
   Temperature (<100.4° F.)
   Weight (no restriction)
For a subject with child-bearing potential
   Discuss the risks involved in becoming pregnant while receiving vaccine
   Ask which birth-control method she will be using while participating in the vaccine trial; FDA acceptable forms include sterilization, implantable rod, IUD, shot/injection, oral contraceptives, barrier methods (vaginal ring, condom, diaphragm, cervical cap), and emergency contraception
Perform colposcopy
   Obtain ThinPrep for HPV-DNA testing
   Obtain punch biopsy and endocervical curettage if determined to be necessary by the physician (HSIL needs to be confirmed to be eligible)
   Physician may acquire four-quadrant blind biopsy if no areas of lesions are visible upon colposcopy
   Record the lesion(s), locations on the cervix, image cervix using the colposcope-mounted image capture system (if available), and indicate where biopsy was taken
   Record in how many cervical quadrants the lesions are visible
   If the subject has already been diagnosed with HSIL by biopsy, there is no need to repeat it. However, colposcopy could be repeated to document the location of the lesion(s), and to collect ThinPrep for HPV-DNA testing.
Draw blood tubes for CBC, hepatic function, and renal function (to be performed in

### Vaccination Visits (Visits 1-5) 6.5

### 6.5.1 Procedures for Visit 1

Ask if any medications have been started or stopped since the last visit
Urine pregnancy test prior to vaccination
Measure height and weight to determine BMI
Take vital signs prior to injection
Blood will be drawn for
   Immunomonitoring and other analyses (six to eight 10.0 ml rubber green top sodium heparin tubes)
   CBC (one 3.0 ml purple top EDTA tube; to be performed in UAMS clinical laboratory)
   Hepatic and renal panels (two 4.5 ml light green top lithium heparin tubes; to be performed in UAMS clinical laboratory)
Administer vaccination injection
Repeat vital signs after at least 30 min has passed since the injection
Monitor for any immediate adverse reactions
Offer dose of ibuprofen or naproxen
Hand out “Subject Diary” (Appendix 4) and ask the subject to fill it out and bring it back at the next visit

### 6.5.2 Procedures for Visit 2

Ask for the filled out “Subject Diary”. If the subject did not return it, ask “Have you experienced any side effects since the last injection?”
Ask if any medications have been started or stopped since the last visit
Urine pregnancy test prior to vaccination
Take vital signs prior to injection
Blood will be drawn for
   Immunomonitoring and other analyses (six to eight 10.0 ml rubber green top sodium heparin tubes)
Administer vaccination injection
Repeat vital signs after at least 30 min has passed since the injection
Monitor for any immediate adverse reactions
Offer dose of ibuprofen or naproxen
Hand out “Subject Diary” (Appendix 4) and ask the subject to fill it out and bring it back at the next visit

### 6.5.3 Procedures for Visit 3

Ask for the filled out “Subject Diary”. If the subject did not return it, ask “Have you experienced any side effects since the last injection?”
Ask if any medications have been started or stopped since the last visit
Urine pregnancy test prior to vaccination
Take vital signs prior to injection
Blood will be drawn for
   Immunomonitoring and other analyses (six to eight 10.0 ml rubber green top sodium heparin tubes)
   CBC (one 3.0 ml purple top EDTA tube; to be performed in UAMS clinical laboratory)
   Hepatic and renal panels (two 4.5 ml light green top lithium heparin tubes; to be performed in UAMS clinical laboratory)
Administer vaccination injection
Repeat vital signs after at least 30 min has passed since the injection
Offer dose of ibuprofen or naproxen
Monitor for any immediate adverse reactions
Hand out “Subject Diary” (Appendix 4) and ask the subject to fill it out and bring it back at the next visit

### 6.5.4 Procedures for Visit 4

Ask for the filled out “Subject Diary”. If the subject did not return it, ask “Have you experienced any side effects since the last injection?”
Ask if any medications have been started or stopped since the last visit
Urine pregnancy test prior to vaccination
Take vital signs prior to injection
Blood will be drawn for
   Immunomonitoring and other analyses (six to eight 10.0 ml rubber green top sodium heparin tubes)
Administer vaccination injection
Repeat vital signs after at least 30 min has passed since the injection
Monitor for any immediate adverse reactions
Offer dose of ibuprofen or naproxen
Hand out “Subject Diary” (Appendix 4) and ask the subject to fill it out and bring it back at the next visit

### 6.5.5 Procedures for Visit 5

Ask for the filled out “Subject Diary”. If the subject did not return it, ask “Have you experienced any side effects since the last injection?”
Blood will be drawn for
   Immunomonitoring and other analyses (six to eight 10.0 ml rubber green top sodium heparin tubes)
   CBC (one 3.0 ml purple top EDTA tube)
   Hepatic and renal panels (two 4.5 ml light green top lithium heparin tubes)

### 6-Month Visit 6.6

The 6-Month visit will be scheduled approximately six months (±2 weeks) after Vaccination Visit 4.

### 6.6.1 Procedures for 6-Month Visit

Ask if any medications have been started or stopped since last visit
Perform colposcopy
   Obtain ThinPrep for HPV-DNA testing
   Record the lesion(s), locations on the cervix, image cervix using the colposcope-mounted image capture system (if available)
   Record in how many cervical quadrants the lesions are visible
   If determined to be necessary by the physician (ONLY in cases where there is a suspicion of progressive disease), obtain punch biopsy and endocervical curettage
Based on the results of the ELISPOT assay, some subjects will be further studied for cross-reactivity, epitope spreading and/or defining novel T-cell epitopes, and blood will be drawn
   Six to eight 10.0 ml rubber green top sodium heparin tubes

### 12-Month Visit 6.7

The 12-Month visit will be scheduled approximately six months (±2 weeks) after the 6-Month visit.

### 6.7.1 Procedures for 12 Month Visit

Perform a physical examination
   Obtain vital signs
      Blood pressure
      Heart rate
      Respiratory rate
      Temperature
      Weight
   Ask if any medications have been started or stopped since last visit
Perform colposcopy
   Obtain ThinPrep for HPV-DNA testing
   Record the lesion(s), locations on the cervix, image cervix using the colposcope-mounted image capture system (if available)
   Record in how many cervical quadrants the lesions are visible
   Obtain at least one punch biopsy from each of the 4 quadrants and possibly endocervical curettage
      Obtain at least one biopsy from each quadrant with visible lesions
      In a quadrant without visible lesions, obtain at least one biopsy from each quadrant described to have had HSIL lesions at the Screening Visit
      In a quadrant without visible lesions and without a record of having had HSIL lesions at the Screening Visit, obtain one blind biopsy
   If determined to be necessary by the physician, perform endocervical curettage
Blood may be drawn from some subjects as explained above for
   Immunomonitoring and other analyses (six to eight 10.0 ml rubber green top sodium heparin tubes)
Have the subject fill out “12 Month Visit Questionnaire” (Appendix 7) during the visit

### 6.7.2 Follow-Up to the 12 Month Visit

The Study Coordinator and Principal Investigator or Co-Investigator will review all information and test results from the 12 Month Visit. If no evidence of HSIL upon biopsy, the subject will complete the study. If persistent HSIL is present, the subject may choose either to (1) be followed by her private gynecologist for another one year prior to LEEP or (2) to have LEEP performed as a part of the study.

### Optional LEEP Visit 6.8

### 6.8.1 Procedures for LEEP Visit

Blood may be drawn from some subjects as explained above for
   Immunomonitoring and other analyses (six to eight 10.0 ml rubber green top sodium heparin tubes)
Perform LEEP biopsy
   Obtain ThinPrep specimen for HPV-DNA testing
   Excise visible lesion or, if no visible lesion seen, excise from an area where biopsy was obtained at the 12-Month Visit

### 8 Outcome Measures

### Clinical Assessments (UAMS Pathology Laboratory) 8.1

Clinical response will be assessed (by Pathologists on service in the Pathology Department) by comparing punch biopsy results from screening (having had HSIL is the inclusion criterion) with the punch biopsy performed at the 12 Month visit. The subject will be considered a “responder” if the 12 Month biopsy is negative for HSIL (no evidence of CIN 2/3), or a “non-responder” if the biopsy shows HSIL (CIN 2 and/or 3).

### Virological Study-HPV-DNA Testing (Nakagawa Laboratory) 8.2

The ThinPrep samples will be tested for the presence of HPV-DNA. A commercially available kit such as the “Linear Array HPV Genotyping Test” may be used (Roche Molecular Diagnostics, Inc., Alameda, Calif.). This kit tests for 37 HPV types (6, 11, 16, 18, 26, 31, 33, 35, 39, 40, 42, 45, 51, 52, 53, 54, 55, 56, 58, 59, 61, 62, 64, 66, 67, 68, 69, 70, 71, 72, 73, 81, 82, 83, 84, IS39, and CP6108). The human beta-globin signal will also be assayed as a positive control for sample adequacy for DNA content from each sample. Positive-control samples (with added HPV plasmid DNA and plasmid-encoded human beta-globin gene) and negative-control samples (no HPV plasmid DNA and no human beta-globin gene) are provided by the manufacturer and will be included in each experiment. HPV types 31, 33, 35, 52, 58, and 67 will be considered “HPV 16-Related”, additionally HPV types 18, 39, 45, 51, 53, 56, 59, 66, 68, 69, 70, 73, and 82 will be considered “High Risk”, and types 6, 11, 40, 42, 54, 61, 62, 71, 72, 81, 83, 84, and CP6108 will be considered “Low Risk”[58].

The virological response will be assessed by comparing HPV-DNA testing results before and after vaccination. The subject will be considered a “clearer” if at least one HPV type(s) present before vaccination becomes undetectable at both 6-Month and 12-Month Visits. Otherwise, a subject will be considered a “persistor” as long as at least one HPV type was detected at baseline.

### Immunological Assessments 8.3

### 8.3.1 ELISPOT Assay (Nakagawa Laboratory)

An immune assay such as an ELISPOT assay to assess the presence of HPV-specific T-cells will be performed. After each blood draw, PBMCs will be separated into CD14+ and CD14− populations and cryopreserved. To eliminate interassay variability, all three blood samples (before vaccination, after two vaccinations, and after four vaccinations) will be used to establish T-cell lines and to perform ELISPOT assays. CD3 T-cell lines will be established by stimulating in vitro magnetically selected CD3 cells with autologous mature dendritic cells exposed to HPV 16 E6-vac, E7-vac, and E6-GST. ELISPOT assays will be performed as previously described[28]. We typically examine 10 regions within the HPV 16 E6 and E7 proteins (E6 1-25, E6 16-40, E6 31-55, E6 46-70, E6 61-85, E6 76-100, E6 91-115, E6 106-130, E6 121-145, E6 136-158). The assay will be performed in triplicate if sufficient cells are available. In order to compare each region before vaccination and after 2 or 4 injections, a t test for paired samples will be performed, as described previously[59]. Therefore, each subject will be assessed in terms of the number of regions with statistically significant increased T-cell responses after two injections or four injections determined by using Student's paired t-test. Remaining CD3 T-cells may be used to assess the recognition of homologous epitopes from other high-risk HPV types, to describe novel epitopes, and/or to assess the endogenous processing of such epitopes.

### 8.3.2 Measuring Immune Cells

### 8.3.2.1 Circulating Immune Cells (Nakagawa Laboratory)

A small amount of PBMCs (approximately 3×106 cells) from blood draws at Visit 1, Visit 3, and Visit 5 will also be used to monitor levels of circulating immune cells such as Tregs and MDSC to assess whether vaccination may decrease their levels[60]. Flow cytometry will be used to determine the number of CD4+ CD25+ FOXP3+ (Treg)[29] and CD11b+CD14+CD33+IL4Rα+HLA-DRint/neg (MDSC) cells[61, 62]. Tbet (Th1), GATA3 (Th2), and/or ROR gammaT (TH17) positive cells may also be examined. The number of circulating immune cells will be determined before vaccination, after two, and after four injections.

### 8.3.2.2 Cervical Immune Cells (UAMS Experimental Pathology Core)

After routine pathological diagnosis has been made from LEEP sample obtained at the Optional LEEP Visit, additional sections may be examined for cervical immune cells such as those positive for CD3 (T-cell), CD4 (helper T-cell), CD8 (cytotoxic T-cell), CD56 (NK cell), CD1a (Langerhan cells important in antigen presentation), CD20 (B-cell), CD68 (macrophage), FOXP3 (Treg), Tbet (Th1), and MadCAM-1 (addressing involved with T-cell infiltration). Eosinophils (Th2) may also be examined.

### 8.3.3 Others

Additional analyses that may be performed using blood samples to assess vaccine response include antibody production to HPV proteins, cytokine responses (Nakagawa laboratory), and changes in protein expression (UAMS Proteomics Core Laboratory).

### 9 Data Analysis

### Assessing Efficacy 9.1

A historical placebo group, from a previously reported study with a similar study design (i.e., enrollment of subjects with biopsy-proven CIN2/3, and clinical response assessed by biopsy in 15 months), will be used for comparison[57]. The response rate in PeCan or CANDIN recipients who completed the trial will be compared with that of the historical placebo group which was 29% (34 of 117) using Fisher's exact test. The response rates between the PepCan and CANDIN groups will also be compared. See “Rationale for Primary Outcome Measure: Efficacy” (Section 1.5.9) for power analysis and sample size justification.

### Assessing Safety: Summary of Adverse Effects 9.2

Subjects who received at least one dose of PepCan or CANDIN will be included in safety assessments. Results will be tabulated as shown in Table 4. The type of adverse reactions, the CTCAE grades, and whether the reactions are vaccine-related will be indicated.

### Assessing Immunological Response and Viral Clearance 9.3

### 9.3.1 Immunological Response

### 9.3.1.1 CD3 T-Cell Response to HPV

As described above, a paired t-test for paired samples will be performed in order to compare each region with increased positivity index after 2 or 4 injections compared to pre-vaccination for the PepCan arm. An analogous analysis will be performed for the CANDIN arm, and the number of regions with statistically significant increases will be compared between the two treatment arms to elucidate the additive effects of the E6 peptides.

A correlation between CD3 T-cell response to HPV and clinical response will be examined by drawing a contingency table for a number of subjects with at least one region with statistically significant increase to E6 in “responders” and “non-responders”. Fisher's exact test will be used.

### 9.3.1.2 Circulating Immune Cells

The changes in percentage of circulating immune cells such as CD4, Th1, Th2, Treg, and MDSC will be compared after 2 vaccinations, 6 months after 4 vaccinations, and 12 months after 4 vaccinations with baseline as shown in FIG. 6. Paired t-test and one-way ANOVA will be performed to determine statistical significance separately for the PepCan and CANDIN groups.

The differences between the percentages of each circulating immune cell types will be compared between the “responders” and the “non-responders” at pre-vaccination, post-2 vaccination, 6 months after post-4 vaccination, and 12 months after post-4 vaccination using Wilcoxon rank-sum test separately for the Pepcan and CANDIN groups.

### 9.3.2 Viral Clearance

HPV-DNA testing will performed using Thin-Prep samples from Screening, 6 Month, and 12 Month Visits.

A correlation between clinical response and virological response (at least one HPV type becoming undetectable after vaccination) will be examined by drawing a contingency table for responder vs. non-responders and HPV persistence vs. HPV clearance separately for the Pepcan and CANDIN groups. Fisher's exact test will be used.

### Factors Contributing to Study Recruitment and Retention 9.4

Based on data provided in “Screening Visit Questionnaire”, “Early Termination Questionnaire”, and “12 Month Visit Questionnaire”, factors that contribute to subject recruitment and retention may be assessed. The Fisher's exact test will be used to compare factors such as frequent use of Facebook private group, motivation for entering the study, or having young children will be compared between the subjects who exited the study early and the subjects who completed the study.

### Factors Predicting Clinical Response and Viral Clearance 9.5

Because proteomics data will be collected at 3 time points, we will identify clusters of proteins which are associated with specific dynamic responses to vaccine (e.g. increasing, decreasing, U-shaped) and also identify protein-expression signatures which predict vaccine response. Protein clustering will be performed using Mfuzz[62], a noise-robust clustering method originally developed for gene expression microarray time-course data, but which has been successfully applied to proteomics data[63]. We will test protein clusters for enrichment of specific gene ontology (GO) annotations to elucidate underlying causes of differential response to vaccine. In addition to proteomics data, we will test other variables for prediction of vaccine response, first by univariate analyses, and then multivariable analysis with variable selection using lasso[64] with ten-fold cross validation. Computations will be performed in the R and R/Bioconductor[65] environments. Variable selection using lasso will be implemented with the package glmmLasso, while enrichment analysis for Gene Ontology terms will be performed using topGO.

### Definitions 10.1

### 10.1.1 Adverse Event

An adverse event is any occurrence or worsening of an undesirable or unintended sign, symptom, or disease that is temporally associated with the use of the vaccine, and it will be graded according to the Common Terminology Criteria for Adverse Events (CTCAE) Version 4.03. Local and/or systemic adverse events may include itching, burning, pain, peeling, rash, oozing, redness, tenderness, scarring, fever, nausea, dizziness, and wheezing. The subjects will be allowed to use and provided analgesics (such as ibuprofen or naproxen) according to the appropriate dosages after injections to limit any adverse events that may occur. Any adverse event will be reviewed and considered related or not related to the vaccine. All applicable events will be reported to the IRB according to IRB policy 10.2 and the FDA according to 21 CFR 312.32.

### 10.1.2 Serious Adverse Event

A serious adverse event is any medical event that
Results in death
Is an immediate threat to life
Requires hospitalization or prolongation of existing hospitalization
Is a congenital anomaly or birth defect, or
Other important medical events that have not resulted in death, are not life-threatening, or do not require hospitalization, may be considered serious adverse events when, based upon the appropriate medical judgment, they are considered to jeopardize the subject and may require medical or surgical intervention to prevent one of the outcomes listed above.

### Results

It is believed that both the CANDIN alone and the PepCan will result in an increase in systemic Th1 levels and will result in regression of HPV lesions. It is believed that both arms of the study will have a larger proportion than the proportion of historical untreated controls become negative for HSIL after the treatment course.

### References for Example 4

[1] Cancer Facts & Figures. American Cancer Society; 2012.
[2] Crum C. Robbins & Cotran Pathologic Basis of Disease. 7th Edition ed. Philadelphia London Toronto Montreal Sydney Tokyo: W. B. Saunders Co., 2004.
[3] Munoz N, Bosch F X, de Sanjose S, Herrero R, Castellsague X, Shah K V, Snijders P J, Meijer C J. Epidemiologic classification of human papillomavirus types associated with cervical cancer. N Engl J Med. 2003; 348:518-27.
[4] Beaudenon S, Kremsdorf D, Croissant O, Jablonska S, Wain-Hobson S, Orth G. A novel type of human papillomavirus associated with genital neoplasias. Nature. 1986; 321:246-9.
[5] Crum C P, Mitao M, Levine R U, Silverstein S. Cervical papillomaviruses segregate within morphologically distinct precancerous lesions. J Virol. 1985; 54:675-81.
[6] Reid R. Human papillomaviral infection. The key to rational triage of cervical neoplasia. Obstet Gynecol Clin North Am. 1987;14:407-29.
[7] Lorincz A T, Lancaster W, Temple G. Cloning and characterization of the DNA of a new human papillomavirus from a woman with dysplasia of the uterine cervix. J Virol. 1986; 58:225-9.
[8] Lorincz A T, Quinn A, Lancaster W, Temple G F. A new type of papillomavirus associated with cancer of the uterine cervix. Virology. 1987; 159:187-190.
[9] Fuchs P G, Girardi F, Pfister H. Human papillomavirus DNA in normal, metaplastic, preneoplastic and neoplastic epithelia of the cervix uteri. Int J Cancer. 1988; 41:41-5.
[10] Kiviat N B, Koutsky L A, Critchlow C W, Lorincz A T, Cullen A P, Brockway J, Holmes K K. Prevalence and cytologic manifestations of human papilloma virus (HPV) types 6, 11, 16, 18, 31, 33, 35, 42, 43, 44, 45, 51, 52, and 56 among 500 consecutive women. Int J Gynecol Pathol. 1992; 11:197-203.
[11] Richart R M, Barron B A. A follow-up study of patients with cervical dysplasia. Am J Obstet Gynecol. 1969; 105:386-93.
[12] Nash J D, Burke T W, Hoskins W J. Biologic course of cervical human papillomavirus infection. Obstet Gynecol. 1987; 69:160-2.
[13] Campion M J, McCance D J, Cuzick J, Singer A. Progressive potential of mild cervical atypia: prospective cytological, colposcopic, and virological study. Lancet. 1986; 2:237-40.
[14] Massad LS, Einstein M H, Huh W K, Katki H A, Kinney W K, Schiffman M, Solomon D, Wentzensen N, Lawson H W. 2012 updated consensus guidelines for the management of abnormal cervical cancer screening tests and cancer precursors. Obstet Gynecol. 2013; 121:829-46.
[15] Bruinsma F J, Quinn M A. The risk of preterm birth following treatment for precancerous changes in the cervix: a systematic review and meta-analysis. BJOG. 2011; 118:1031-41.
[16] Nieminen P, Harper D M, Einstein M H, Garcia F, Donders G, Huh W, Wright T C, Stoler M, Ferenczy A, Rutman O, Shikhman A, Leung M, Clinch B, Calleja E. Efficacy and safety of RO5217990 treatment in patients with high grade cervical intraepithelial neoplasia (CIN2/3). 28th International Papillomavirus Conference. Puerto Rico 2012.
[17] Daayana S, Elkord E, Winters U, Pawlita M, Roden R, Stern P L, Kitchener H C. Phase II trial of imiquimod and HPV therapeutic vaccination in patients with vulval intraepithelial neoplasia. Br J Cancer. 2010; 102:1129-36.
[18] Kenter G G, Welters M J, Valentijn A R, Lowik M J, Berends-van der Meer D M, Vloon A P, Essahsah F, Fathers L M, Offringa R, Drijfhout J W, Wafelman A R, Oostendorp J, Fleuren G J, van der Burg S H, Melief C J. Vaccination against HPV-16 oncoproteins for vulvar intraepithelial neoplasia. N Engl J Med. 2009; 361:1838-47.
[19] Stanley M, Pinto L A, Trimble C. Human papillomavirus vaccines—immune responses. Vaccine. 2012; 30 Suppl 5:F83-7.
[20] Moscicki A B, Palefsky J M, Gonzales J, Smith G, Schoolnik G K. Colposcopic and histologic findings and human papillomavirus (HPV) DNA test variability in young women positive for HPV DNA. J Infect Dis. 1992; 166:951-7.
[21] Moscicki A B, Palefsky J, Smith G, Siboski S, Schoolnik G. Variability of human papillomavirus DNA testing in a longitudinal cohort of young women. Obstet Gynecol. 1993; 82:578-85.
[22] Moscicki A B, Shiboski S, Broering J, Powell K, Clayton L, Jay N, Darragh T M, Brescia R, Kanowitz S, Miller S B, Stone J, Hanson E, Palefsky J. The natural history of human papillomavirus infection as measured by repeated DNA testing in adolescent and young women. J Pediatr. 1998; 132:277-84.
[23] Nakagawa M, Stites D P, Farhat S, Sisler J R, Moss B, Kong F, Moscicki A B, Palefsky J M. Cytotoxic T lymphocyte responses to E6 and E7 proteins of human papillomavirus type 16: relationship to cervical intraepithelial neoplasia. J Infect Dis. 1997; 175:927-31.
[24] Nakagawa M, Stites D P, Palefsky J M, Kneass Z, Moscicki A B. CD4-Positive and CD8-positive cytotoxic T lymphocytes contribute to human papillomavirus type 16 E6 and E7 responses. Clin Diagn Lab Immunol. 1999; 6:494-8.
[25] Nakagawa M, Stites D P, Patel S, Farhat S, Scott M, Hills N K, Palefsky J M, Moscicki A B. Persistence of human papillomavirus type 16 infection is associated with lack of cytotoxic T lymphocyte response to the E6 antigens. J Infect Dis. 2000; 182:595-8.
[26] Farhat S, Nakagawa M, Moscicki A-B. Cell-mediated immune responses to human papillomavirus 16 E6 and E7 antigens as measured by interferon gamma enzyme-linked immunospot in women with cleared or persistent human papillomavirus infection. International Journal of Gynecological Cancer. 2009; 19:508-12.
[27] Nakagawa M, Gupta S K, Coleman H N, Sellers M A, Banken J A, Greenfield W W. A favorable clinical trend is associated with CD8 T-cell immune responses to the human papillomavirus type 16 e6 antigens in women being studied for abnormal pap smear results. J Low Genit Tract Dis. 2010; 14:124-9.
[28] Nakagawa M, Kim K H, Moscicki A B. Patterns of CD8 T-cell epitopes within the human papillomavirus type 16 (HPV 16) E6 protein among young women whose HPV 16 infection has become undetectable. Clin Diagn Lab Immunol. 2005; 12:1003-5.
[29] Kim K H, Greenfield W W, Cannon M J, Coleman H N, Spencer H J, Nakagawa M. CD4+ T-cell response against human papillomavirus type 16 E6 protein is associated with a favorable clinical trend. Cancer Immunol Immunother. 2012; 61:63-70.
[30] Wang X, Coleman H N, Nagarajan U, Spencer H J, Nakagawa M. Candida skin test reagent as a novel adjuvant for a human papillomavirus peptide-based therapeutic vaccine. Vaccine. 2013; 31:5806-13.
[31] Audran R, Cachat M, Lurati F, Soe S, Leroy O, Corradin G, Druilhe P, Spertini F. Phase I malaria vaccine trial with a long synthetic peptide derived from the merozoite surface protein 3 antigen. Infect Immun. 2005; 73:8017-26.
[32] Celis E. Overlapping human leukocyte antigen class I/II binding peptide vaccine for the treatment of patients with stage IV melanoma: evidence of systemic immune dysfunction. Cancer. 2007; 110:203-14.
[33] Elliott S L, Suhrbier A, Miles J J, Lawrence G, Pye S J, Le T T, Rosenstengel A, Nguyen T, Allworth A, Burrows SR, Cox J, Pye D, Moss D J, Bharadwaj M. Phase I trial of a CD8+ T-cell peptide epitope-based vaccine for infectious mononucleosis. J Virol. 2008; 82:1448-57.
[34] Hueman M T, Dehqanzada Z A, Novak T E, Gurney J M, Woll M M, Ryan G B, Storrer C E, Fisher C, McLeod D G, Ioannides C G, Ponniah S, Peoples G E. Phase I clinical trial of a HER-2/neu peptide (E75) vaccine for the prevention of prostate-specific antigen recurrence in high-risk prostate cancer patients. Clin Cancer Res. 2005; 11:7470-9.
[35] Kenter G G, Welters M J, Valentijn A R, Lowik M J, Berends-van der Meer D M, Vloon A P, Drijfhout J W, Wafelman A R, Oostendorp J, Fleuren G J, Offringa R, van der Burg S H, Melief C J. Phase I immunotherapeutic trial with long peptides spanning the E6 and E7 sequences of high-risk human papillomavirus 16 in end-stage cervical cancer patients shows low toxicity and robust immunogenicity. Clin Cancer Res. 2008; 14:169-77.
[36] Muderspach L, Wilczynski S, Roman L, Bade L, Felix J, Small L A, Kast W M, Fascio G, Marty V, Weber J. A phase I trial of a human papillomavirus (HPV) peptide vaccine for women with high-grade cervical and vulvar intraepithelial neoplasia who are HPV 16 positive. Clin Cancer Res. 2000; 6:3406-16.
[37] Roberts J D, Niedzwiecki D, Carson W E, Chapman P B, Gajewski T F, Ernstoff M S, Hodi F S, Shea C, Leong S P, Johnson J, Zhang D, Houghton A, Haluska F G. Phase 2 study of the g209-2M melanoma peptide vaccine and low-dose interleukin-2 in advanced melanoma: Cancer and Leukemia Group B 509901. J Immunother. 2006; 29:95-101.
[38] Suekane S, Nishitani M, Noguchi M, Komohara Y, Kokubu T, Naitoh M, Honma S, Yamada A, Itoh K, Matsuoka K, Kanayama H. Phase I trial of personalized peptide vaccination for cytokine-refractory metastatic renal cell carcinoma patients. Cancer Sci. 2007; 98:1965-8.
[39] Gupta R K. Aluminum compounds as vaccine adjuvants. Adv Drug Deliv Rev. 1998; 32:155-72.
[40] Esch R E, Buckley C E, 3rd. A novel Candida albicans skin test antigen: efficacy and safety in man. J Biol Stand. 1988; 16:33-43.
[41] Clifton M M, Johnson S M, Roberson P K, Kincannon J, Horn T D. Immunotherapy for recalcitrant warts in children using intralesional mumps or Candida antigens. Pediatr Dermatol. 2003; 20:268-71.
[42] Horn T D, Johnson S M, Helm R M, Roberson P K. Intralesional immunotherapy of warts with mumps, Candida, and Trichophyton skin test antigens: a single-blinded, randomized, and controlled trial. Arch Dermatol. 2005; 141:589-94.
[43] Johnson S M, Horn T D. Intralesional immunotherapy for warts using a combination of skin test antigens: a safe and effective therapy. J Drugs Dermatol. 2004; 3:263-5.
[44] Johnson S M, Roberson P K, Horn T D. Intralesional injection of mumps or Candida skin test antigens: a novel immunotherapy for warts. Arch Dermatol. 2001; 137:451-5.
[45] Phillips R C, Ruhl T S, Pfenninger J L, Garber M R. Treatment of warts with Candida antigen injection. Arch Dermatol. 2000; 136:1274-5.
[46] Maronn M, Salm C, Lyon V, Galbraith S. One-year experience with candida antigen immunotherapy for warts and molluscum. Pediatr Dermatol. 2008; 25:189-92.
[47] Kim K H, Horn T D, Pharis J, Kincannon J, Jones R, O'Bryan K, Myers J, Nakagawa M. Phase 1 clinical trial of intralesional injection of Candida antigen for the treatment of warts. Arch Dermatol. 2010; 146:1431-3.
[48] Nakagawa M, Coleman H N, Wang X, Daniels J, Sikes J, Nagarajan U M. IL-12 secretion by Langerhans cells stimulated with Candida skin test reagent is mediated by dectin-1 in some healthy individuals. Cytokine. 2014; 65:202-9.
[49] Peng S, Ji H, Trimble C, He L, Tsai Y C, Yeatermeyer J, Boyd D A, Hung C F, Wu T C. Development of a DNA vaccine targeting human papillomavirus type 16 oncoprotein E6. J Virol. 2004; 78:8468-76.
[50] Abbas A K, Lichtman A H, Pillai S. Cellular and Molecular Immunology. 7th ed. Philadelphia: Elsevier, 2011.
[51] Hildesheim A, Herrero R. Effect of a HPV-16/18 vaccine on resolution of of infections in women with pre-existing HPV. 23rd International Papillomavirus Conference & Clinical Workshop. Prague, Czech Republic 2006: 6.
[52] Ault K A. Effect of prophylactic human papillomavirus L1 virus-like-particle vaccine on risk of cervical intraepithelial neoplasia grade 2, grade 3, and adenocarcinoma in situ: a combined analysis of four randomised clinical trials. Lancet. 2007; 369:1861-8.
[53] Pedersen C, Petaja T, Strauss G, Rumke H C, Poder A, Richardus J H, Spiessens B, Descamps D, Hardt K, Lehtinen M, Dubin G. Immunization of early adolescent females with human papillomavirus type 16 and 18 L1 virus-like particle vaccine containing AS04 adjuvant. J Adolesc Health. 2007; 40: 564-71.
[54] Jemal A, Simard E P, Dorell C, Noone A M, Markowitz L E, Kohler B, Eheman C, Saraiya M, Bandi P, Saslow D, Cronin K A, Watson M, Schiffman M, Henley S J, Schymura M J, Anderson R N, Yankey D, Edwards B K. Annual Report to the Nation on the Status of Cancer, 1975-2009, featuring the burden and trends in human papillomavirus (HPV)-associated cancers and HPV vaccination coverage levels. J Natl Cancer Inst. 2013; 105:175-201.
[55] Horn T, Johnson S, Roberson P. Intralesional immunotherapy of warts with mumps, Candida and trichophyton skin test antigens: a single-blinded, randomized and controlled trial. Archives of Dermatology. 2005.
[56] Kenter G, Welters M J, Valentijn A R, Lowik M J, Berends-van der Meer D M, Vloon A P, Offringa R, Drijfhout J W, Wafelman A R, Oostendorp J, Fleuren G J, van der Burg S H, Melief C J. Vaccination against HPV-16 Oncoproteins for Vulvar Intraepitheial Neoplasia. N Engl J Med. 2009; 361:1838-47.
[57] Meyskens F L, Jr., Surwit E, Moon T E, Childers J M, Davis J R, Dorr R T, Johnson C S, Alberts D S. Enhancement of regression of cervical intraepithelial neoplasia II (moderate dysplasia) with topically applied all-trans-retinoic acid: a randomized trial. J Natl Cancer Inst. 1994; 86:539-43.
[58] de Villiers E M, Fauquet C, Broker T R, Bernard H U, zur Hausen H. Classification of papillomaviruses. Virology. 2004; 324:17-27.
[59] Kim K H, Greenfield W, Shotts E, Nakagawa M. Detection of human papillomavirus type 16-specific T lymphocytes by a recombinant vaccinia virus-based enzyme-linked immunospot assay. Clin Vaccine Immunol. 2007; 14:362-8.
[60] Pasare C, Medzhitov R. Toll pathway-dependent blockade of CD4+CD25+ T cell-mediated suppression by dendritic cells. Science. 2003;299:1033-6.
[61] Greten T F, Manns MP, Korangy F. Myeloid derived suppressor cells in human diseases. Int Immunopharmacol. 2011; 11:802-7.
[62] Futschik M E, Carlisle B. Noise-robust soft clustering of gene expression time-course data. J Bioinform Comput Biol. 2005; 3:965-88.
[63] Krahmer N, Hilger M, Kory N, Wilfling F, Stoehr G, Mann M, Farese R V, Jr., Walther T C. Protein correlation profiles identify lipid droplet proteins with high confidence. Mol Cell Proteomics. 2013; 12:1115-26.
[64] Tibshirani R. Shrinkage and selection via the Lasso. Journal of the Reoyal Statistical Society Series B. 1996; 58:267-88.
[65] Gentleman R C, Carey V J, Bates D M, Bolstad B, Dettling M, Dudoit S, Ellis B, Gautier L, Ge Y, Gentry J, Hornik K, Hothorn T, Huber W, Iacus S, Irizarry R, Leisch F, Li C, Maechler M, Rossini A J, Sawitzki G, Smith C, Smyth G, Tierney L, Yang J Y, Zhang J. Bioconductor: open software development for computational biology and bioinformatics. Genome Biol. 2004; 5:R80.

### Example 5

### A Phase I Clinical Trial of CANDIN Alone and of a Mixture of CANDIN and E6 Peptides (PepCan) to Prevent Recurrence of Head and Neck Cancer

A phase I human clinical trial will be conducted in head and neck cancer patients who have had their cancers go into complete remission. The trial will be a double-blind placebo-controlled trial with patients randomized to receive intradermal injection of 300 ul of normal saline or of 300 ul of CANDIN or a mixture of 300 ul of CANDIN and 100 ug of each of the E6 peptides described in Example 1. They will be dosed with four injections spaced 3 weeks apart, and then three injections spaced 3 months apart, (i.e., at weeks 0, 3, 6, and 9, and then at weeks 22, 35, and 48). Patients will be clinically observed for one year.

The level of circulating immune cells, including CD4 T-cells, Th1 cells, Th2 cells, regulatory T-cells (Treg), and myeloid-derived suppressor cells (MDSC), will be assessed before vaccination, after 2 vaccinations, after 4 vaccinations, and at one year. Data from the Phase I clinical trial in Example 2 above indicate that the CANDIN-peptides mixture (PepCan) may increase Th1 responses (p=0.02) and decrease Th2 responses resulting in increased effector immune activity (FIGS. 6A and 6B). Whether the levels of these circulating immune cells can be used to predict vaccine efficacy in terms of preventing recurrence will be investigated.

### Brief Description of the Study

The main purpose of the study would be to assess the safety of administering 7 PepCan injections. In a previous clinical trial, 4 injections were given to 34 subjects with no dose-limiting toxicity reported. In addition, the magnitude and durability of Th1 shift demonstrated in the previous trial will be further assessed. Twenty subjects with head and neck cancer in remission will be enrolled regardless of their HPV status. The first 4 injections will be given 3 weeks apart, and the next 3 injections will be given 3 months apart. Then, the subjects will be observed for additional 1 year with blood draws at 6 months and exit.

Schedule of Study Visits, Blood Draws and Laboratory Analyses

| | Visits | | | | | | | | |
|---|---|---|---|---|---|---|---|---|---|
| | 1 | 2 | 3 | 4 | 5 | 6 | 7 | 8 | 9 (Exit) |
| Vaccination | X | X | X | X | X | X | X | | |
| Blood draw | X | | X | | X | X | X | X | X |
| 1 Purple* | X | | X | | X | X | X | X | X |
| 1 Light green* | X | | X | | X | X | X | X | X |
| 2 Rubber Green | | | X | | | X | X | X | |
| 8 Rubber Green | X | | | | X | | | | X |
| FACS (Th1, Th2, Treg) | X | | X | | X | X | X | X | X |
| HPV 16 E6 ELISPOT | X | | | | X | | | | X |
| Cytokine/chemokine\hat{ } | X | | | | | | | | |

*The purple top tube is for CBC, and the light green top tube is for hepatic and renal panels.
\hat{ }44 plasma cytokine/chemokines to be measured to identify biomarkers for vaccine response: IL-1β, IL-1 receptor agonist (IL-1RA), IL-2, IL-4, IL-5, IL-6, IL-7, IL-8, IL-9, IL-10, IL-12 (p70), IL-13, IL-15, IL-17A, eotaxin, basic fibroblast growth factor (FGF), G-CSF, GM-CSF, IFN-γ, IFN-γ induced protein 10 (IP-10), monocyte chemotactic protein 1 (MCP-1), MIP-1α, MIP-1β, platelet-derived growth factor subunit B (PDGF-BB), regulated on activation, normal T-cell expressed and secreted (RANTES), TNF-α, vascular endothelial growth factor (VEGF), IL-2 receptor α (IL-2Rα), chemokine (C-X-C motif) ligand 1 (CXCL1), hepatocyte growth factor (HGF), IFN-α2, LIF, chemokine (C-C motif) ligand 6 (CCL7), macrophage migration inhibitory factor (MIF), chemokine (C-X-C motif) ligand 9 (CXCL9), β-nerve growth factor (β-NGF), stem cell factor (SCF), stem cell growth factor β (SCGF-β), TRAIL, IL-16, and IL-18.

All publications, patents, and patent documents cited are hereby incorporated by reference.

What is claimed is:

1. A method of stimulating a systemic T helper cell type 1 response in a mammal in need thereof, the method comprising:
injecting a composition comprising a recall antigen intradermally in a mammal in need thereof;
wherein the method is not a method of treating a herpes simplex virus infection;
and wherein the method does not comprise injecting a composition comprising a recall antigen intradermally into a viral epithelial lesion;
wherein the method increases T helper cell type 1 response in the mammal; and
(i) wherein the mammal is infected with a microorganism and afflicted with a disease caused by the microorganism, and the composition comprising a recall antigen does not comprise an antigen of the microorganism infecting the mammal; or
(ii) wherein the mammal is afflicted with a cancer or was afflicted with a cancer and the cancer is now in remission, and the composition comprising a recall antigen does not comprise an antigen of the cancer currently or previously afflicting the mammal.

2. The method of claim 1 wherein the recall antigen is Candida extract, mumps antigen, or Trichophyton extract.

3. The method of claim 1 wherein the recall antigen stimulates IL-12 secretion from Langerhans cells in vitro.

4. The method of claim 1 wherein the mammal is a human and the method comprises injecting the recall antigen intradermally in the human at a dose level and on a dose schedule, wherein the recall antigen increases Th1 cells in most humans receiving intradermal injection of the recall antigen at the dose level and dose schedule.

5. The method of claim 1 wherein the mammal is a human infected with HPV and afflicted with a disease caused by HPV.

6. The method of claim 1 wherein the mammal is afflicted with a cancer or was afflicted with a cancer and the cancer is now in remission, and the composition comprising a recall antigen does not comprise an antigen of the cancer currently or previously afflicting the mammal;
wherein the cancer is caused by HPV.

7. The method of claim 1 wherein wherein the mammal is afflicted with a cancer or was afflicted with a cancer and the cancer is now in remission, and the composition comprising a recall antigen does not comprise an antigen of the cancer currently or previously afflicting the mammal;
wherein the cancer is cervical cancer, head and neck cancer, vulvar cancer, anal cancer, vaginal cancer, or penile cancer.

8. The method of claim 1 further comprising administering an immunological checkpoint inhibitor to the mammal.

9. The method of claim 8 wherein the immunological checkpoint inhibitor is an anti-PD-1 antibody, an anti-PDL1 antibody, or an anti-CTLA-4 antibody.

10. The method of claim 1 wherein the mammal is afflicted with cancer, the method further comprising administering an anti-PD-1 antibody or an anti-CTLA-4 antibody to the mammal.

11. The method of claim 1 wherein the mammal is a human.

12. The method of claim 1 wherein the mammal is a dog or cat or a mouse or rat.

13. A method of treating a microbial infection or cancer in a mammal comprising:
injecting a composition comprising a recall antigen intradermally in a mammal in need thereof;
wherein the method is not a method of treating a herpes simplex virus infection; and wherein the method does not comprise injecting a composition comprising a recall antigen intradermally into a viral epithelial lesion; and
(i) wherein the mammal is infected with a microorganism and afflicted with a disease caused by the microorganism, and the composition comprising a recall antigen does not comprise an antigen of the microorganism infecting the mammal; or
(ii) wherein the mammal is afflicted with a cancer or was afflicted with a cancer and the cancer is now in remission, and the composition comprising a recall antigen does not comprise an antigen of the cancer currently or previously afflicting the mammal.

14-35. (canceled)

36. A method of stimulating a systemic T helper cell type 1 response in a mammal in need thereof, the method comprising:
injecting a composition comprising a recall antigen intradermally in a mammal in need thereof;
wherein the method is not a method of treating a herpes simplex virus infection;
and wherein the method does not comprise injecting a composition comprising a recall antigen intradermally into a viral epithelial lesion;
wherein the method increases T helper cell type 1 response in the mammal; and
wherein the mammal was afflicted with a cervical cancer, head and neck cancer, vulvar cancer, anal cancer, vaginal cancer, penile cancer, or a cancer caused by HPV and the cancer is now in remission.

37-39. (canceled)

40. A method of preventing growth of tumors or recurrence of cancer in a mammal comprising:
injecting a composition comprising a recall antigen intradermally in a mammal in need thereof;
wherein the method is not a method of treating a herpes simplex virus infection;
and wherein the method does not comprise injecting a composition comprising a recall antigen intradermally into a viral epithelial lesion;
wherein the method increases T helper cell type 1 response in the mammal; and
wherein the mammal is afflicted with cervical cancer or head and neck cancer or a cancer caused by HPV, or the mammal was afflicted with cervical cancer or head and neck cancer or a cancer caused by HPV and the cancer is now in remission.

41. The method of claim 40 wherein the composition further comprises HPV E6 protein or a plurality of peptide fragments of HPV E6 protein of 10-100 amino acid residues in length, the fragments collectively comprising at least 50% of SEQ ID NO:1.

42. The method of claim 41 wherein the composition comprises peptides consisting of residues 1-45, 46-80, 81-115, and 116-158 of SEQ ID NO:1.

43. The composition of claim 41 wherein the composition comprises peptide fragments of HPV E6 and the peptides are acetylated on their amino termini or amidated on their carboxy termini, or acetylated on their amino termini and amidated on their carboxy termini.

44. The method of any one of claim 40 wherein the mammal was afflicted with cervical cancer or head and neck cancer or a cancer caused by HPV, and the cancer is now in remission, and the method is a method of preventing recurrence of the cancer.

45. The method of any one of claim 42 wherein the mammal was afflicted with cervical cancer or head and neck cancer or a cancer caused by HPV, and the cancer is now in remission, and the method is a method of preventing recurrence of the cancer.


SEQUENCE LISTING (5 sequences)

SEQ ID NO: 1 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 2 - protein, 45 aa, Human papillomavirus type 16
MHQKRTAMFQDPQERPRKLPQLCTELQTTIHDIILECVYCKQQLL

SEQ ID NO: 3 - protein, 35 aa, Human papillomavirus type 16
RREVYDFAFRDLCIVYRDGNPYAVCDKCLKFYSKI

SEQ ID NO: 4 - protein, 35 aa, Human papillomavirus type 16
SEYRHYCYSLYGTTLEQQYNKPLCDLLIRCINCQK

SEQ ID NO: 5 - protein, 43 aa, Human papillomavirus type 16
PLCPEEKQRHLDKKQRFHNIRGRWTGRCMSCCRSSRTRRETQL